(19) United States
(12) Patent Application Publication
(10) Pub. No.: US 2023/0238143 A1
(43) Pub. Date: Jul. 27, 2023

(54) MULTIMODALITY SYSTEMS AND METHODS FOR DETECTION, PROGNOSIS, AND MONITORING OF NEUROLOGICAL INJURY AND DISEASE

(71) Applicant: BRAINBOX SOLUTIONS, INC., Richmond, VA (US)

(72) Inventors: Donna J. EDMONDS, Richmond, VA (US); Timothy E. VAN METER, Richmond, VA (US); Nazanin MIRSHAHI, Richmond, VA (US); Allison KUMAR, Richmond, VA (US)

(21) Appl. No.: 18/002,517

(22) PCT Filed: Jun. 23, 2021
(86) PCT No.: PCT/US2021/038774

§ 371 Date: Dec. 20, 2022

Related U.S. Application Data
(60) Provisional application No. 63/042,985, filed on Jun. 23, 2020.

(51) Int. Cl. G16H 50/30; G16H 10/20; G16H 50/20; G16H 40/67; G16B 25/10

(52) U.S. Cl. CPC G16H 50/30; G16H 10/20; G16H 50/20; G16H 40/67; G16B 25/10

(57) ABSTRACT

Systems and methods for determining diagnostic and/or prognostic risk of having or developing brain injury related symptoms after a head impact includes a point-of-care assay reader, a PHI smart device application, and a neurocognitive/vestibular smart device application. The diagnosis and prognosis server application includes instructions stored on a non-transitory computer-readable medium executed on the server that receives patient protected health information (PHI) from the PHI smart device application, receives neurocognitive test results from a neurocognitive testing application, and receives assay results from the point-of-care assay reader and generates a diagnostic score and a prognostic risk scores for post-acute traumatic brain injury TBI symptom categories as measures of patient outcomes.

### CROSS-REFERENCE TO RELATED APPLICATION

This application claims the benefit of priority to U.S. Provisional Application No. 63/042,985 filed on Jun. 23, 2020. The provisional application is incorporated by reference in its entirety in this application.

### BACKGROUND

Traumatic brain injury (TBI) is caused by a head injury that can result in lasting damage to the brain. TBI affects up to 10 million patients worldwide each year. The health effects of TBI can be debilitating, result in long term disability, and have significant financial burdens.

TBI is graded as mild (defined by no more than a brief change in mental status or loss of consciousness for less than 30 seconds), moderate, or severe (meaning an extended period of unconsciousness or amnesia after the injury) on the basis of the level of consciousness or Glasgow coma scale (GCS) score after resuscitation. The GCS score includes the sum of eye opening (spontaneous=4, to speech=3, to pain=3, none=1), motor response (obeys=6, localizes=5, withdraws=4, abnormal flexion=3, extensor response=2, none=1), and verbal response (oriented=5, confused=4, inappropriate=3, incomprehensible=2, none=1). Mild TBI (GCS 13-15) is in most cases a concussion, and there is full neurological recovery, although many of these patients have short-term memory and concentration difficulties. In moderate TBI (GCS 9-13) the patient is lethargic or stuporous, and in severe injury (GCS 3-8) the patient is comatose, unable to open his or her eyes or follow commands.

Patients with severe TBI have a significant risk of hypotension, hypoxemia, and brain swelling. If these sequelae are not prevented or treated properly, they can exacerbate brain damage and increase the risk of death.

The term “traumatic intracerebral hemorrhage” as used in this description refers to such bleeding that is caused, caused by, or associated with traumatic injury. Intracerebral hemorrhages commonly occur in the basal ganglia, thalamus, brain stem (predominantly the pons), cerebral hemispheres, and the cerebellum. Extension into the ventricles occurs in association with deep, large hematomas. Edematous parenchyma, often discolored by degradation products of hemoglobin, is visible adjacent to the clot. Histologic sections are characterized by the presence of edema, neuronal damage, macrophages, and neutrophils in the region surrounding the hematoma. The hemorrhage spreads between planes of white-matter cleavage, causing some destruction of the brain structure, and leaving intact neural tissue within and surrounding the hematoma.

Intraparenchymal bleeding results from the rupture of the small penetrating arterioles that originate from basilar arteries or from the anterior, middle, or posterior cerebral arteries. Degenerative changes in the arteriolar walls by chronic hypertension reduce compliance, weaken the wall, and increase the likelihood of spontaneous rupture. Studies suggest that most bleeding occurs at or near the bifurcation of affected arteries, where prominent degeneration of the tunica media and smooth muscles can be seen.

Neurological damage after TBI does not all occur immediately at the moment of impact (primary injury), but instead evolves afterwards (secondary injury). Secondary brain injury is the leading cause of in-hospital deaths after TBI. Most secondary brain injury is caused by brain swelling, with an increase in intracranial pressure and a subsequent decrease in cerebral perfusion leading to ischemia. Within hours of TBI, due to a breakdown of tight endothelial junctions which make up the blood-brain barrier (BBB), normally excluded intravascular proteins and fluid penetrate into cerebral parenchymal extracellular space (vasogenic edema). Once plasma constituents cross the BBB, the edema spreads. The vasogenic fluid accumulating in brain causes cerebral edema, raises intracranial pressure, and lowers the threshold of systemic blood pressure for cerebral ischemia. A reduction in cerebral blood flow or oxygenation below a threshold value or increased intracranial pressure leading to cerebral herniation increases brain damage and morbidity.

Approximately 10% of TBIs (1,400,000 annual U.S. cases) are complicated by intracerebral hemorrhage requiring surgery. The delay in the breakdown of the blood-brain barrier and the development of cerebral edema after an intracerebral hemorrhage (ICH) suggest that there may be secondary mediators of both neural injury and edema. It is generally believed that blood and plasma products mediate most secondary processes that are initiated after an ICH.

Clinical tools such as physical exam, and central nervous system (CNS) imaging (computerized tomography (CT) scan or magnetic resonance imaging (MRI)) are subjective, not widely available, not sensitive or specific enough, and too costly to identify all patients with CNS injury, and therefore have a high false negative rate. A need exists to quickly identify patients having or at high risk of developing an intracerebral hemorrhage so that they can receive surgery or other medical intervention on an urgent basis, and to separate them from patients who can be managed conservatively or safely discharged.

An estimated 5 million patients per year are evaluated for head injury in the emergency departments of US institutions per year, most of which have unclear clinical symptoms and no evidence of damage by CT imaging of the head. Objective tests are needed that can quickly triage patients at high and low risk for neurological injury-related symptoms. Several biomarkers have been reported in the scientific literature, but single and multiple biomarker models have been insufficient to predict clinical course or recommend treatments.

### SUMMARY

A group of optimized methods and algorithms constructed in accordance with the principles and exemplary embodiments of invention are integrated by combining: 1) specific functional testing metrics from a custom-designed neurocognitive testing app; 2) objective detection of the combined and accumulative brain physiological biomarkers in biofluids; and 3) the age and sex of the individual, to detect neurological injury and to predict long-term and emergent symptoms. The systems and methods according to exemplary embodiments of the invention provide a customized approach to solving an important clinical problem in distinguishing TBI subjects from non-TBI subjects. Point of care application of the systems and methods according to the principles and exemplary embodiments of the invention in a rapid multimodality testing system with an integrated algorithmic result provide significant benefits that could not be anticipated by the sum of its individual parts. Methods and algorithms constructed in accordance with the principles and exemplary embodiments of the invention draw informative data from a combination of clinical subject attributes (age and sex), blood biomarker levels from a panel of proteins released from the brain upon injury, and several individual metrics from a digital motor and cognitive function testing platform (app). Metrics for motor and cognitive function performance are included as inputs for exemplary embodiments of the methods and algorithms of the invention and include balance testing, ocular motor tracking, convergence insufficiency, neurocognitive tasks (memory, pattern finding, reasoning tasks, processing speed), and other motor performance and cognitive performance metrics.

Exemplary embodiments of the invention provide systems and methods for stratifying patients and identifying those having an intracranial bleed or those who have a high, moderate, or low risk for developing an intracranial bleed that requires urgent medical intervention. In one example embodiment of the invention, a testing system integrates multiple testing input types into a multipart algorithm, that utilizes specific input data from blood tests and a unique blend of subject characteristics and brain function testing applications (apps), and that distinguishes patterns of biofluid protein levels and interactive app-based performance metrics from a subject, and compares the pattern of the subject to established normative databases from control subjects, to diagnose brain injury and provide a risk assessment for adverse outcomes (post-brain injury symptoms).

Exemplary system embodiments of the invention include a multi-analyte assay of brain injury biomarkers in a biological sample taken from an individual known to have, or suspected of having a brain injury, or from an individual to determine baseline levels of the biomarkers. For example, a blood test assays may be a multiplex immunoassay assessing multiple discrete biomarkers in a multiplex panel. A point of care reader integrates the assays into a point of care device that is portable and provides rapid results. The tests can be performed on a large analyzer and non-point-of-care readings can be transported and included with the blood test results. Exemplary systems also include digital cognitive tests that are integrated on hand-held or other computing devices to provide a rapid, mobile, and user-friendly neurocognitive-motor testing assessment of the patients. Exemplary systems then process the results of the blood test/multiplex analyte assay, the neurocognitive-motor testing, and patient health information, including protected health information (PHI), such as age, sex, and other health information. Exemplary systems then process these results and automatically generate a brain injury likelihood score and a risk score-based prognosis report.

In an example embodiment of the invention, the system components provide elements of data that are used as inputs for the algorithm. More specifically, exemplary methods for diagnosing brain injury in a patient include (a) receiving multiplex analyte test results from the patient; (b) receiving PHI related to the patient; (c) receiving neurocognitive-motor testing results for the patient; (d) automatically generating a diagnostic score and prognostic risk scores for post-acute TBI symptom categories as measures of likelihood of patient outcomes; and e) diagnosing brain injury based on the automatically generated diagnostic score and prognostic risk scores.

In more specific embodiments, the automatically generated diagnostic score and prognostic risk scores correlate to one or more brain injury statuses selected from the group of having a traumatic brain injury (TBI), intracranial hemorrhage, having intraparenchymal hemorrhage, sub-acute brain injury, acute brain injury, post-acute brain injury, progressing brain injury, regressing brain injury, subclinical brain injury, mild brain injury, moderate brain injury, severe brain injury and chronic brain injury, wherein a correlation to one of the predefined levels determines the brain injury status of the subject. In an embodiment, hemorrhage in a subject may be an intraparenchymal hemorrhage or an intraventricular hemorrhage.

Exemplary systems and methods of the invention generate a quantitative interpretation of test results derived from these measurements in conjunction with computerized neurological assessments to provide a diagnosis and a prognosis report of a patient's likelihood of post-injury symptom occurrence.

One example embodiment of the invention includes a system for determining prognostic risk of having or developing brain injury related symptoms after a head impact. The system includes a diagnosis and prognosis server application, including instructions stored on a non-transitory computer-readable medium executed on the server. The diagnosis and prognosis server application is configured to receive patient protected health information (PHI) from a PHI smart device application, neurocognitive test results from a neurocognitive testing application, and assay results from a point-of-care assay reader. The diagnosis and prognosis server application is configured to generate a diagnostic score and a prognostic risk scores for post-acute TBI symptom categories as measures of patient outcomes.

Exemplary embodiments of diagnosis and prognosis server applications of the invention can be configured to generate the diagnostic score based on predetermined classification criteria differentiating states of brain injury and normal conditions based on the assay results, the neurocognitive test results, and the PHI. Similarly, exemplary systems of the invention including the diagnosis and prognosis server applications is configured to generate the prognosis risk score based on predetermined classification criteria differentiating states of brain injury and normal conditions based on the assay results, the neurocognitive test results, and the PHI.

Additionally, exemplary diagnosis and prognosis servers of the invention is configured to generate a risk strata classification of the patient, where the risk strata classification includes at least one of the group of: i) No TBI-Normal; ii) TBI positive low risk for post-acute symptoms; and iii) TBI positive high risk for post-acute symptoms. Similarly, exemplary systems of the invention including the diagnosis and prognosis server applications is configured to generate a risk strata classification of the patient, where the risk strata classification includes a categorized time frame associated with the determined risk strata classification of the patient. The risk strata classifications of the patient can include an outcome category, and the outcome category can include headache, motor deficit, sleep disturbance, cognitive deficit, and/or psychological affect. Further, diagnosis and prognosis applications constructed in accordance with exemplary embodiments of the invention can be configured to generate a recommended treatment intervention based on the risk strata classification and the predetermined time frame. The recommended treatment interventions can include at least one of: i) a pain medication regimen; ii) physical therapy; iii) vision therapy; iv) sleep therapy; v) cognitive therapy; and vi) psychotherapy.

Exemplary embodiments of the invention can also include neurocognitive test results that include metrics of brain function and performance including metrics of at least one of the group of: balance testing, oculomotor tracking, convergence insufficiency, neurocognitive memory tasks, neurocognitive pattern finding tasks, neurocognitive reasoning tasks, and neurocognitive processing speed tasks. Also, the neurocognitive test results can include metrics of brain function and performance that include validated screening metrics of at least one of the group of headache, motor deficit, sleep disturbance, cognitive function, and psychological state.

Example embodiments of the invention can be configured to include neurocognitive testing applications that load at least one of the group of a digitized patient questionnaire and a motor function testing procedure whose answers form the basis for the neurocognitive test results. The neurocognitive testing applications can include instructions stored on a non-transitory computer-readable medium executed on a smart device that is located in a geographically distinct location from a location of the BRAINBox server, and the smart device can be connected to the BRAINBox server via a communications network. In some embodiments of the invention, the geographically distinct location is outside a hospital, an outpatient treatment site, and an urgent care facility, and a telemedicine user interface can be used to connect to the BRAINBox server.

In some embodiments of the invention, the system can include a normative database of at least one of the group of patient assay test results, patient protected health information (PHI), patient neurocognitive test results, and patient vestibular and motor test results, and the diagnosis and prognosis server application can generate the diagnostic score based on predetermined classification criteria differentiating states of brain injury and normal conditions based on the at least one of the group of patient assay test results, patient protected health information (PHI), patient neurocognitive test results, and patient vestibular and motor test results in the normative database. The predetermined classification criteria differentiating states of brain injury and normal conditions can includes at least one of Random Forest, logistic regression, logit boost, and extreme gradient boosting.

Likewise, in some embodiments of the invention, the system can include a normative database of at least one of the group of patient assay test results, patient protected health information (PHI), patient neurocognitive test results, and patient vestibular and motor test results and the diagnosis and prognosis server application generates the prognosis risk score based on predetermined classification criteria differentiating states of brain injury and normal conditions based on the at least one of the group of patient assay test results, patient protected health information (PHI), patient neurocognitive test results, and patient vestibular and motor test results in the normative database. The predetermined classification criteria differentiating states of brain injury and normal conditions can include at least one of Random Forest, logistic regression, logit boost, and extreme gradient boosting.

In some embodiments of the invention, the system includes a multi-analyte assay to detect, and optionally, measure levels of, one or more biomarkers in a biofluid sample obtained from a patient having or suspected of having traumatic brain injury (TBI), the assay being integrated in a point of care device to generate first input signals representative of the levels of the one or more biomarkers.

The system can also include a computer processor configured to receive digital neurocognitive, vestibular and/or oculomotor function inputs for the patient. The inputs can include one or more metrics of brain function and performance. The computer processor can also be configured to generate second input signals for an algorithm, including one or more of balance testing, oculomotor tracking, convergence insufficiency, and specific neurocognitive tasks, including one or more of memory, pattern finding, reasoning tasks, processing speed. The system also includes a first classifying algorithm to differentiate states of brain injury and normal condition using the first and second input signals, together with subject age and sex as covariates, to classify the patient into a TBI or No TBI category. The system also includes a second stratification algorithm to place a patient determined to be in the TBI category into one of at least two of the following three risk strata: i) TBI positive low risk for one or more post-acute symptoms, ii) TBI positive moderate/medium risk for one or more post-acute symptoms, and iii) TBI positive high risk for one or more post-acute symptoms.

In some embodiments of the invention, the biomarkers are one or more of: proteins; lipids; nucleic acids; and metabolites of proteins, lipids, or nucleic acids.

In some embodiments of the invention, the one or more protein biomarkers comprise Aldolase C (ALDOC), Brain derived neurotrophic factor (BDNF), Calcitonin Gene Related Peptide (CGRP), Endothelin 1 (ET1), Eotaxin (CCL11), Fatty Acid Binding Protein 7 (FABP7), Glial Fibrillary Acidic Protein (GFAP), Growth Associated Protein 43 (GAP-43), Intercellular Adhesion Molecule 5 (ICAM-5), Interleukin 6 (IL-6), Interleukin 8 (IL-8), Interleukin 10 (IL-10), Interleukin-33 (IL-33), Metallothionein 3 (MT3), Neurogranin (NRGN), Neurofilament heavy chain (NF-H), Neurofilament light chain (NF-L), Neurofilament medium chain (NF-M), Neuron Specific Enolase (ENO2/NSE), calcium binding protein S100B, Oligodendrocyte Myelin Glycoprotein (OMG), Reticulon (RTN1), Synuclein alpha (SNCA), Synuclein beta (SNCB), Tau microtubule binding protein (TAU/MAPT), von Willebrand Factor (vWF), Vascular Endothelial Growth Factor (VEGF-A, B, C or D homo or heterodimers), post-translational modifications thereof, fragments thereof, auto-antibodies thereof.

In some embodiments of the invention, the one or more protein biomarkers further comprise one or more of: brain lipid binding protein (BLBP/FABP7); a trauma-specific break down product (BDP) of ALDOC or BLBP/FABP7; glutamine synthetase (GS), astrocytic phosphoprotein PEA-15 (PEA15), αB-crystallin (CRYABiHSP27), a trauma-specific proteolytic cleavage product of ALDOC, GS, PEA 15, CRYAB, and a 20-30 kDa BDP of GFAP.

In some embodiments of the invention, the one or more protein biomarkers further comprise one of more of the protein biomarkers, post-translational modifications thereof, fragments thereof, auto-antibodies thereof listed in FIG. 5.

In some embodiments of the invention, the one or more biomarkers comprise: (i) a cell adhesion protein, a cell signaling protein, a cell toxicity protein, a clotting protein, a cytoskeleton protein, an extracellular matrix protein, a gene expression mediating protein, a gene regulation protein, an inflammation protein, a microtubule trafficking protein, a lipid binding protein, a metabolic enzyme, a metabolism protein, a protein-binding protein, a proteolytic protein, a signaling protein, a structural protein, a synapse protein; (ii) at least one protein biomarker found in mammalian cells or tissue, selected from a protein found in astrocytes, one or more proteins found in blood, one or more protein found in blood, heart and liver tissue, one or more proteins found in brain tissue, a protein found in cardiac tissue, a protein found in epithelial tissue, a protein found in interneurons, a protein found in neuroepithelial cells, one or more proteins found in neurons, a protein found in skin tissue, one or more ubiquitous proteins, and combinations thereof; (iii) a protein with a role in a brain repair process selected from one or more apoptosis proteins, one or more inflammation proteins, one or more innate immunity proteins, one or more membrane repair proteins, one or more metabolism proteins, one or more necrosis proteins, one or more neurodegeneration proteins, one or more neurogenesis proteins, one or more synaptogenesis proteins, one or more vascular repair proteins; or (iv) combinations of (i), (ii), and (iii).

In some embodiments of the invention, the one or more biomarkers comprise a subset of biomarkers comprising at least one of: Protein No.12, Astrotactin 2 (ASTN2); Protein No. 30, Cullin-7 (CUL7); Protein No. 50, metallothionein 1 isoform X (MT1X); Protein No. 67, Slit-Robo GTPase protein (SRGAP1); and Protein No. 79, von Willebrand Factor (vWF).

In some embodiments of the invention, the subset of biomarkers further comprises one or more of Brain-Derived Neurotrophic Factor (BDNF); Glial Fibrillary Acidic Protein (GFAP); Intracellular Adhesion Molecule 5 (ICAM5); Synuclein Beta (SNCB); Metallothionein 3 (MT3); Neurogranin (NRGN); Neuron Specific Enolase (NSE); and Aldolase C (ALDOC).

In some embodiments of the invention, one or more of the biomarkers are citrullinated, acetylated, methylated, dimethylated, carboxylated, sumoylated, or phosphorylated.

In some embodiments of the invention, the system is utilized to determine the prognostic risk of having or developing brain injury related symptoms after a head impact.

In some embodiments of the invention, the system is utilized to monitor patient prognosis or recovery at a remote location outside a hospital, outpatient site, or urgent care clinical settings using internet connected computer or internet connected mobile device interfaces.

In some embodiments of the invention, the system is utilized in a home-based telemedicine application.

In some embodiments of the invention, the digital neurocognitive, vestibular and/or oculomotor function input is derived from an integrated software application to perform digitized questionnaires, neurocognitive testing, vestibular and motor function testing procedures according to a preloaded protocol.

In some embodiments of the invention, the digital neurocognitive, vestibular and/or oculomotor function inputs performed on the Internet connected computer or internet connected mobile device are compared to a normative database, adjusted for the subject's age and sex to generates time-dependent change metrics.

In some embodiments of the invention, the second stratification algorithm is configured to renders a predetermined category classification and one or more associated prognostic risk scores using a predetermined algorithmic model.

In some embodiments of the invention, the bio-sample is one or more of: a blood sample, a serum sample, a plasma sample, a cerebrospinal fluid (CSF) sample, a nasal fluid sample, a saliva sample, a urine sample, a sputum sample, a secretion sample, a tear sample, a sweat sample, or an organ tissue sample.

Some embodiments of the invention include a method of testing a patient having or suspected of having traumatic brain injury (TBI). In some example embodiments of the invention, the method includes receiving results of a bio-sample obtained from the patient from a multi-analyte biomarker assay; receiving neurocognitive, vestibular and/or oculomotor functional testing results from the patient; comparing in a processor the results from the multi-analyte biomarker assay to a normative sample of results from the multi-analyte biomarker assay; comparing in a processor the results from the neurocognitive, the results from vestibular and/or the results from the oculomotor functional testing to a normative sample of results from the neurocognitive, the results from vestibular and/or the results from the oculomotor functional testing; differentiating states of brain injury and normal condition using the results from the multi-analyte biomarker assay and the results from the neurocognitive, the results from vestibular and/or the results from the oculomotor functional testing, together with subject age and sex as covariates, in a first classifying algorithm to classify the patient into a TBI or No TBI category; and calculating a TBI score in a second stratification algorithm to place a patient determined to be in the TBI category into one of at least two of the following three risk strata: i) TBI positive low risk for one or more post-acute symptoms, ii) TBI positive moderate/medium risk for one or more post-acute symptoms, and iii) TBI positive high risk for one or more post-acute symptoms.

In some embodiments of the invention, the methods include receiving other patient health information (PHI), and using these data as additional features in a processor that are inputs to the first or second algorithm, or both, and comparing in a processor the other patient health information to a normative sample of patient health information, prior to being used as additional features in the one or more algorithms.

In some embodiments of the invention, the biomarkers are one or more of: proteins; lipids; nucleic acids; and metabolites of proteins, lipids, or nucleic acids.

In some embodiments of the invention, the multi-analyte biomarker assay tests for the presence of one or more of the following protein biomarkers selected from the group consisting of: Aldolase C (ALDOC), Brain derived neurotrophic factor (BDNF), Calcitonin Gene Related Peptide (CGRP), Endothelin 1 (ET1), Eotaxin (CCL11), Fatty Acid Binding Protein 7 (FABP7), Glial Fibrillary Acidic Protein (GFAP), Growth Associated Protein 43 (GAP-43), Intercellular Adhesion Molecule 5 (ICAM-5), Interleukin 6 (IL-6), Interleukin 8 (IL-8), Interleukin 10 (IL-10), Interleukin-33 (IL-33), Metallothionein 3 (MT3), Neurogranin (NRGN), Neurofilament heavy chain (NF-H), Neurofilament light chain (NF-L), Neurofilament medium chain (NF-M), Neuron Specific Enolase (ENO2/NSE), Oligodendrocyte Myelin Glycoprotein (OMG), Reticulon (RTN1), Synuclein alpha (SNCA), Synuclein beta (SNCB), Tau microtubule binding protein (TAU/MAPT), von Willebrand Factor (vWF), Vascular Endothelial Growth Factor (VEGF-A, B, C or D homo or heterodimers), post-translational modifications thereof, fragments thereof, auto-antibodies thereof, or combinations thereof, or combinations thereof.

In some embodiments of the invention, the protein biomarkers further comprise one or more of: brain lipid binding protein (BLBP/FABP7); a trauma-specific break down product (BDP) of ALDOC or BLBP/FABP7; glutamine synthetase (GS), astrocytic phosphoprotein PEA-15 (PEA15), αB-crystallin (CRYABiHSP27), a trauma-specific proteolytic cleavage product of ALDOC, GS, PEA 15, CRY AB, and a 20-30 kDa BDP of GFAP.

In some embodiments of the invention, the protein biomarkers further comprise one of more of the protein biomarkers, post-translational modifications thereof, fragments thereof, auto-antibodies thereof listed in FIG. 5.

In some embodiments of the invention, the biomarkers comprise: (i) a cell adhesion protein, a cell signaling protein, a cell toxicity protein, a clotting protein, a cytoskeleton protein, an extracellular matrix protein, a gene expression mediating protein, a gene regulation protein, an inflammation protein, a microtubule trafficking protein, a lipid binding protein, a metabolic enzyme, a metabolism protein, a protein-binding protein, a proteolytic protein, a signaling protein, a structural protein, a synapse protein; (ii) at least one protein biomarker found in mammalian cells or tissue, selected from a protein found in astrocytes, one or more proteins found in blood, one or more protein found in blood, heart and liver tissue, one or more proteins found in brain tissue, a protein found in cardiac tissue, a protein found in epithelial tissue, a protein found in interneurons, a protein found in neuroepithelial cells, one or more proteins found in neurons, a protein found in skin tissue, one or more ubiquitous proteins, and combinations thereof; (iii) a protein with a role in a brain repair process selected from one or more apoptosis proteins, one or more inflammation proteins, one or more innate immunity proteins, one or more membrane repair proteins, one or more metabolism proteins, one or more necrosis proteins, one or more neurodegeneration proteins, one or more neurogenesis proteins, one or more synaptogenesis proteins, one or more vascular repair proteins; or (iv) combinations of (i), (ii), and (iii).

In some embodiments of the invention, the one or more biomarkers listed in FIG. 5 comprise a subset of biomarkers comprising at least one of: Protein No.12, Astrotactin 2 (ASTN2); Protein No. 30, Cullin-7 (CUL7); Protein No. 50, metallothionein 1 isoform X (MT1X); Protein No. 67, Slit-Robo GTPase protein (SRGAP1); and Protein No. 79, von Willebrand Factor (vWF).

In some embodiments of the invention, the subset of biomarkers further comprises one or more of Brain-Derived Neurotrophic Factor (BDNF); Glial Fibrillary Acidic Protein (GFAP); Intracellular Adhesion Molecule 5 (ICAM5); Synuclein Beta (SNCB); Metallothionein 3 (MT3); Neurogranin (NRGN); Neuron Specific Enolase (NSE); and Aldolase C (ALDOC).

In some embodiments of the invention, one or more of the protein biomarkers are citrullinated, acetylated, methylated, dimethylated, carboxylated, sumoylated, or phosphorylated.

In some embodiments of the invention, the functional testing results includes at least one of the group of reaction time, cognitive processing, visual attention, task switching, executive function, memory, balance testing, oculomotor tracking, and convergence insufficiency.

In some embodiments of the invention, the neurocognitive test results is based upon the results of patient tasks associated with memory, pattern finding, reasoning, or processing speed.

In some embodiments of the invention, the methods further comprise collecting patient health information (PHI) from other patient physiological evaluations as additional features that are inputs to the first or second algorithm, or both.

In some embodiments of the invention, the risk strata are associated with TBI symptoms including post-traumatic headaches, motor deficit, sleep disturbance, seizures, depression, anxiety, loss of cognitive function, or post-traumatic stress disorders.

In some embodiments of the invention, the multi-analyte assay comprises a point-of-care device.

In some embodiments of the invention, the neurocognitive, vestibular and/or oculomotor functional testing results are derived from an integrated software application to perform digitized questionnaires, neurocognitive testing, and motor function testing procedures according to a preloaded protocol.

In some embodiments of the invention, the bio-sample is one or more of: a blood sample, a serum sample, a plasma sample, a cerebrospinal fluid (CSF) sample, a saliva sample, a urine sample, a sputum sample, a secretion sample, a tear sample, a sweat sample, or an organ tissue sample.

In some embodiments of the invention, the system is utilized to determine the prognostic risk of having or developing brain injury related symptoms after a head impact. Exemplary systems of the invention can also be used to monitor patient prognosis or recovery at a remote location outside a hospital, outpatient site, or urgent care clinical settings using Internet connected computers or Internet connected mobile device interfaces. For example, exemplary systems of the invention can be utilized in a home-based telemedicine application.

In example embodiments of the invention the digital neurocognitive, vestibular and/or oculomotor function input is derived from an integrated software application to perform digitized questionnaires, neurocognitive testing, vestibular and motor function testing procedures according to a preloaded protocol. The digital neurocognitive, vestibular and/or oculomotor function inputs can be performed on the Internet connected computer or Internet connected mobile device and compared to a normative database, adjusted for the subject's age and sex to generate time-dependent change metrics. Additionally, some of the systems in accordance with the invention include a second stratification algorithm that is configured to render a predetermined category classification and one or more associated prognostic risk scores using a predetermined algorithmic model.

Some example embodiments of the invention include a method of testing a patient having or suspected of having traumatic brain injury (TBI). The method includes receiving results of from a multi-analyte biomarker assay from a subject's bio-sample; and receiving neurocognitive testing results, vestibular testing results, and/or oculomotor functional testing results from the subject. The methods further include comparing in a processor the respective results from the multi-analyte biomarker assay to a normative sample of results from the multi-analyte biomarker assay and comparing in a processor the results from the neurocognitive, the results from vestibular, and/or the results from the oculomotor functional testing to a normative sample of results from the neurocognitive, vestibular, and/or oculomotor functional testing. The method then differentiates states of brain injury and normal condition using the results from the multi-analyte biomarker assay and the results from the neurocognitive, vestibular and/or oculomotor functional testing, together with subject age and sex as covariates, in a first classifying algorithm to classify the patient into a TBI or No TBI category. The method calculates a TBI score in a second stratification algorithm to place a patient determined to be in the TBI category into one of at least two of the following three risk strata: i) TBI positive low risk for one or more post-acute symptoms, ii) TBI positive moderate/medium risk for one or more post-acute symptoms, and iii) TBI positive high risk for one or more post-acute symptoms.

In some example embodiments of the invention, the methods can include receiving other patient health information (PHI), and using these data as additional features in a processor that are inputs to the first or second algorithm, or both, and comparing in a processor the other patient health information to a normative sample of patient health information, prior to being used as additional features in the one or more algorithms.

In some example embodiments of the invention the functional testing results include at least one of the group of: reaction time; cognitive processing; visual attention; task switching; executive function; memory; balance testing; oculomotor tracking; and convergence insufficiency. In some examples of the invention, the neurocognitive test results are based upon the results of patient tasks associated with memory, pattern finding, reasoning, or processing speed.

Some embodiments of methods of the invention also include collecting patient health information (PHI) from other patient physiological evaluations as additional features that are inputs to the first or second algorithm, or both. In some cases, the risk strata associated with TBI symptoms can include post-traumatic headaches, motor deficit, sleep disturbance, seizures, depression, anxiety, loss of cognitive function, or post-traumatic stress disorders. Additionally, in some examples of the invention, the multi-analyte assay comprises a point-of-care device.

In some examples of the invention, the neurocognitive, vestibular and/or oculomotor functional testing results are derived from an integrated software application to perform digitized questionnaires, neurocognitive testing, and motor function testing procedures according to a preloaded protocol.

Some example embodiments of the invention include a method of building a classification and stratification model for the diagnosis, prognosis and treatment of patients having traumatic brain injury (TBI) or suspected of having traumatic brain injury (TBI). One example method includes receiving, from an assay reader, biomarker test results of a first set of biomarkers from TBI patients having differences in biomarker levels relative to a normative database. The determined differences can be a difference of zero when the biomarker levels are the same as those in the normative database.

In addition, the method includes receiving, from a smart device, functional test results, where the functional test results include at least one of the following test results: neurocognitive, vestibular, and oculomotor functional test results from TBI patients having differences in functional testing results relative to a normative database. As before, the determined differences can be a difference of zero when the biomarker levels are the same as those in the normative database. The method integrates the results of the biomarker test results and the functional testing results, together with subject age and sex, as covariates, to build a first diagnostic algorithm to classify patients into a TBI or No TBI category. Additionally, some example embodiments of the invention build one or more stratification algorithms based upon the classification results of the patient categorized as TBI or No TBI as well as a specific TBI-related symptom. This is used to place patients determined to be in the TBI category into at least one of the following three risk strata: i) TBI positive low risk for one or more post-acute symptoms, ii) TBI positive moderate/medium risk for one or more post-acute symptoms, and iii) TBI positive high risk for one or more post-acute symptoms. In some example embodiments, the invention also is used to treat patients placed in the TBI positive moderate/medium risk or TBI positive high-risk strata.

In some example embodiments of the invention, building the stratification algorithm(s) can include training the second stratification algorithm(s) by determining a weighting of each covariate based upon its ability to predict a change in severity of a TBI-related symptom. The weighting of one of more covariates can be zero.

in some example embodiments of the invention, the TBI-related symptoms that include post-traumatic headaches, motor deficit, sleep disturbance, seizures, depression, anxiety, loss of cognitive function, post-traumatic stress disorders, dizziness, and/or nausea.

Some embodiments of the methods of the invention include cases where the TBI-related symptoms are determined by one of more of the following evaluations: the Rivermead Post-Concussive Symptom Questionnaire (RPQ-16), Generalized Anxiety Disorder Questionnaire 7 questions (GAD-7), Patient Health questionnaire-9 questions (PHQ-9), PTSD Checklist for DSM-5 (PCL-5), Dizziness and Headache Inventory (DHI), Perceived Stress Scale (PSS), Convergence Insufficiency Symptom Survey (CISS), Montreal Cognitive Assessment (MoCA), Mini Mental State Exam (MMSE), Saint Louis University Mental Status Examination (SLUMS), Hopkins Verbal Learning Test-Revised (HVLT-R), and/or Glasgow Outcome Score-extended (GOS-E).

Some example embodiments of the invention utilize the following thresholds for abnormal symptoms: for Rivermead, scores of 3 or more; for GAD-7, scores of 5 or more, with 10 or more being moderate to severe anxiety symptoms; for PHQ-9, scores of 5 or more, with scores of 10 or more being moderate to severe depressive symptoms; for PCL-5, a score of 33 or greater is not recovered (PTSD+); for Dizziness Handicap Inventory (DHI), a score of 16 or more indicates a subject is symptomatic; for Perceived Stress Scale (PSS), a score of 0-13 is low stress, 14-26 is moderate stress symptoms, and 27-40 is high stress symptoms; for Convergence Insufficiency Symptom Survey (CISS), a score of 21 or greater is symptomatic for convergence insufficiency and/or abnormal oculomotor symptoms; for Montreal Cognitive Assessment (MoCA), scores of 25 or less are cognitively impaired; for Mini Mental State Exam (MMSE), a score of 24 or lower is symptomatic for cognitive decline (cognitive deficit); for Saint Louis University Mental Status Examination (SLUMS), a score of 26 or lower indicating cognitive impairment for subjects with at least a high school education and 24 or lower for individuals with less than a high school education; for Hopkins Verbal Learning Test-Revised (HVLT-R), where a score of 14.5 or lower is poor recall, and 24.5 or lower for the memory score indicating poor memory performance; and for Glasgow Outcome Scale-extended (GOS-E), scores of 6 or less are unrecovered, having ongoing disability.

In some example embodiments of the invention, the first set of functional tests results includes at least one test from the group of: reaction time, cognitive processing, visual attention, task switching, executive function, memory, balance testing, oculomotor tracking, and convergence insufficiency. In some example embodiments, the first set of functional test results include neurocognitive test results based upon the results of patient tasks associated with at least one of memory, executive function, pattern finding, reasoning, or processing speed. The neurocognitive test results can be derived from the Flanker test, the Stroop Test, the Digit symbol Substitution Test, the Trailmaking Test, the Trails A and Trails B cognitive and executive function tests, and/or an immediate and delayed recall (short term memory) test.

In some example embodiments, the invention can receive, from a smart device, patient health information (PHI) from a previous patient physiological evaluation; and integrate the PHI as covariates into the first diagnostic algorithm and/or the second stratification algorithms.

In some example embodiments of the invention, the first set of biomarkers are selected from the group consisting of: Aldolase C (ALDOC), Brain derived neurotrophic factor (BDNF), Calcitonin Gene Related Peptide (CGRP), Endothelin 1 (ET1), Eotaxin (CCL11), Fatty Acid Binding Protein 7 (FABP7), Glial Fibrillary Acidic Protein (GFAP), Growth Associated Protein 43 (GAP-43), Intercellular Adhesion Molecule 5 (ICAM-5), Interleukin 6 (IL-6), Interleukin 8 (IL-8), Interleukin 10 (IL-10), Interleukin-33 (IL-33), Metallothionein 3 (MT3), Neurogranin (NRGN), Neurofilament heavy chain (NF-H), Neurofilament light chain (NF-L), Neurofilament medium chain (NF-M), Neuron Specific Enolase (ENO2/NSE), Oligodendrocyte Myelin Glycoprotein (OMG), Reticulon (RTN1), Synuclein alpha (SNCA), Synuclein beta (SNCB), Soluble suppression of tumorigenicity 2 (sST2), Tau microtubule binding protein (TAU/MAPT), von Willebrand Factor (vWF), Vascular Endothelial Growth Factor (VEGF-A, B, C or D homo or heterodimers), post-translational modifications thereof, fragments thereof, auto-antibodies thereof, or combinations thereof.

In some example embodiments of the invention, the first set of biomarkers further includes one or more of: brain lipid binding protein (BLBP/FABP7); a trauma-specific break down product (BDP) of ALDOC or BLBP/FABP7; glutamine synthetase (GS), astrocytic phosphoprotein PEA-15 (PEA15), αB-crystallin (CRYABiHSP27), a trauma-specific proteolytic cleavage product of ALDOC, GS, PEA 15, CRY AB, and a 20-30 kDa BDP of GFAP.

In some example embodiments of the invention, the first set of biomarkers comprises: (i) a cell adhesion protein, a cell signaling protein, a cell toxicity protein, a clotting protein, a cytoskeleton protein, an extracellular matrix protein, a gene expression mediating protein, a gene regulation protein, an inflammation protein, a microtubule trafficking protein, a lipid binding protein, a metabolic enzyme, a metabolism protein, a protein-binding protein, a proteolytic protein, a signaling protein, a structural protein, a synapse protein; (ii) at least one protein biomarker found in mammalian cells or tissue, selected from a protein found in astrocytes, one or more proteins found in blood, one or more protein found in blood, heart and liver tissue, one or more proteins found in brain tissue, a protein found in cardiac tissue, a protein found in epithelial tissue, a protein found in interneurons, a protein found in neuroepithelial cells, one or more proteins found in neurons, a protein found in skin tissue, one or more ubiquitous proteins, and combinations thereof; (iii) a protein with a role in a brain repair process selected from one or more apoptosis proteins, one or more inflammation proteins, one or more innate immunity proteins, one or more membrane repair proteins, one or more metabolism proteins, one or more necrosis proteins, one or more neurodegeneration proteins, one or more neurogenesis proteins, one or more synaptogenesis proteins, one or more vascular repair proteins; or (iv) combinations of one of more (i), (ii), and (iii).

In some example embodiments of the invention, the first set of biomarkers comprises a subset of biomarkers comprising at least one of: Protein No.12, Astrotactin 2 (ASTN2); Protein No. 30, Cullin-7 (CUL7); Protein No. 50, metallothionein 1 isoform X (MT1X); Protein No. 67, Slit-Robo GTPase protein (SRGAP1); and Protein No. 79, von Willebrand Factor (vWF)), and Soluble suppression of tumorigenicity 2 (sST2).

In some example embodiments of the invention, the first set of biomarkers is i) one of more of proteins; lipids; nucleic acids; and metabolites of proteins, lipids, or nucleic acids; ii) citrullinated, acetylated, methylated, dimethylated, carboxylated, sumoylated, or phosphorylated; or iii) combinations of i) and ii).

In some example embodiments of the invention, the biomarker test results are taken from a bio-sample comprising one or more of: a blood sample, a serum sample, a plasma sample, a cerebrospinal fluid (CSF) sample, a saliva sample, a urine sample, a sputum sample, a secretion sample, a tear sample, a sweat sample, and an organ tissue sample.

The above summary is intended to provide an overview of the subject matter described herein and is not intended to identify essential or key elements of the subject matter or to limit the scope of the claimed embodiments, which may be ascertained from the appended claims.

### Definitions

As used herein, the term “antigen” is generally used in reference to any substance that is capable of reacting with an antibody. More specifically, as used herein, the term “antigen” refers to a synthetic peptide, polypeptide, protein or fragment of a polypeptide or protein, or other molecule which elicits an antibody response in a subject or is recognized and bound by an antibody.

As used herein, the term “biomarker” refers to a molecule that is associated either quantitatively or qualitatively with a biological change. Examples of biomarkers include polypeptides, proteins or fragments of a polypeptide or protein; and polynucleotides, such as a gene product, RNA or RNA fragment; and other body metabolites. In certain embodiments, a “biomarker” means a compound that is differentially present (i.e., increased or decreased) in a biological sample from a subject or a group of subjects having a first phenotype (e.g., having a disease or condition) as compared to a biological sample from a subject or group of subjects having a second phenotype (e.g., not having the disease or condition or having a less severe version of the disease or condition). A biomarker may be differentially present at any level, but is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent); or that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more. Alternatively, the differential presence of a biomarker can be characterized by a-fold change in level including, for example, a level that is decreased by 1.1-fold, at least 1.2-fold, at least 1.3-fold, at least 1.4-fold, at least 1.5-fold, at least 2.0-fold, at least 2.5-fold, at least 3.0-fold, at least 3.5-fold, at least 4.0-fold, at least 5-fold, at least 5.5-fold, at least 6-fold, at least 6.5-fold, at least 7.0-fold, at least 7.5-fold, at least 8.0-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 25-fold, at least 30-fold, at least 40-fold, or at least 50-fold; or that is increased by 1.1-fold, at least 1.2-fold, at least 1.3-fold, at least 1.4-fold, at least 1.5-fold, at least 2.0-fold, at least 2.5-fold, at least 3.0-fold, at least 3.5-fold, at least 4.0-fold, at least 5-fold, at least 5.5-fold, at least 6-fold, at least 6.5-fold, at least 7.0-fold, at least 7.5-fold, at least 8.0-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 25-fold, at least 30-fold, at least 40-fold, or at least 50-fold. A biomarker is preferably differentially present at a level that is statistically significant (e.g., a p-value less than 0.05 and/or a q-value of less than 0.10 as determined using, for example, either Welch's T-test or Wilcoxon's rank-sum Test).

The term “one or more of” refers to combinations of various biomarker proteins. The term encompasses 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40 . . . N, where “N” is the total number of biomarker proteins in the particular embodiment. The term also encompasses at least 1, at least 2, at least 3, at least 4, at least 5, at least 6, at least 7, at least 8, at least 9, at least 10, at least 11, at least 12, at least 13, at least 15, 16, 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . N. It is understood that the recitation of biomarkers herein includes the phrase “one or more of” the biomarkers and, in particular, includes the “at least 1, at least 2, at least 3” and so forth language in each recited embodiment of a biomarker panel.

The term “brain injury” refers to a condition in which the brain is damaged by injury caused by an event. As used herein, an “injury” is an alteration in cellular or molecular integrity, activity, level, robustness, state, or other alteration that is traceable to an event. For example, an injury includes a physical, mechanical, chemical, biological, functional, infectious, or other modulator of cellular or molecular characteristics. An event can include a physical trauma such as a single or repetitive impact (percussive) or a biological abnormality such as a stroke resulting from either blockade or leakage of a blood vessel. An event is optionally an infection by an infectious agent. A person of skill in the art recognizes numerous equivalent events that are encompassed by the terms injury or event.

More specifically, the term “brain injury” refers to a condition that results in central nervous system damage, irrespective of its pathophysiological basis. Among the most frequent origins of a “brain injury” are stroke and traumatic brain injury (TBI). A “stroke” is classified into hemorrhagic and non-hemorrhagic. Examples of hemorrhagic stroke include cerebral hemorrhage, subarachnoid hemorrhage, and intracranial hemorrhage secondary to cerebral arterial malformation, while examples of non-hemorrhagic stroke include cerebral infarction.

A distinction is made between intra-axial hemorrhage (blood inside the brain) and extra-axial hemorrhage (blood inside the skull but outside the brain). Intra-axial hemorrhage is due to intra-parenchymal hemorrhage or intra-ventricular hemorrhage (blood in the ventricular system).

In various embodiments, the intra-axial hemorrhage is caused by brain trauma, hemorrhagic stroke and/or spontaneous bleeding into the brain. Likewise, in various embodiments the intraparenchymal hemorrhage, intraventricular hemorrhage, or intraventricular traumatic diffuse bleeding is caused by brain trauma, hemorrhagic stroke and/or spontaneous bleeding into the brain.

The term “traumatic brain injury” or “TBI” refer to traumatic injuries to the brain which occur when physical trauma causes brain damage. For example, TBI can result from a closed head injury or a penetrating head injury. A “non-traumatic brain injury” refers to brain injuries that do not involve ischemia or external mechanical force (e.g., stroke, Alzheimer's disease, Parkinson's disease, Huntington's disease, multiple sclerosis, amyotrophic lateral sclerosis, brain hemorrhage, brain infections, brain tumor, among others).

The term “brain injury” also refers to subclinical brain injury, spinal cord injury, and anoxic-ischemic brain injury. The term “subclinical brain injury” (SCI) refers to brain injury without overt clinical evidence of brain injury. A lack of clinical evidence of brain injury when brain injury actually exists could result from degree of injury, type of injury, level of consciousness, medications particularly sedation and anesthesia.

As used herein, “secondary brain trauma” refers to damage to the brain of a patient post-acute brain injury, i.e., during the secondary injury phase of a TBI.

“Chronic traumatic encephalopathy (CTE)” is a neurodegenerative disease that is most often identified in postmortem autopsies of individuals exposed to repetitive head impacts, such as boxers and football players. The neuropathology of CTE is characterized by the accumulation of hyperphosphorylated tau protein in a pattern that is unique from that of other neurodegenerative diseases, including Alzheimer's disease. The clinical features of CTE are often progressive, leading to dramatic changes in mood, behavior, and cognition, frequently resulting in debilitating dementia. In some cases, motor features, including Parkinsonism, can also be present. Acute traumatic encephalopathy “ATE” is newer term in the TBI field, coined by Applicant's investigators, that refers to the early post-TBI injury-related changes that are the root cause of long term degenerative processes seen in CTE, including neuroinflammatory processes which affect the process of accumulating aggregation of neuronal proteins such as Tau, which are pathological hallmarks of CTE. The specific causative factors arising after injury are still being defined by Applicant and other investigators.

As used herein, “chronic brain injury” refers to a subject who has suffered a brain injury from three months post-injury onward with continuing symptoms from the brain injury.

As used herein, “sub-acute brain injury” refers to a subject who has suffered a brain injury from about 2-5 days post injury.

The “spinal cord injury” refers to a condition in which the spinal cord receives compression/detrition due to a vertebral fracture or dislocation to cause dysfunction. As used herein, the term “anoxic-ischemic brain injury” refers to deprivation of oxygen supply to brain tissue resulting in compromised brain function and includes cerebral hypoxia. For example, anoxic-ischemic brain injury includes focal cerebral ischemia, global cerebral ischemia, hypoxic hypoxia (i.e., limited oxygen in the environment causes reduced brain function, such as with divers, aviators, mountain climbers, and fire fighters, all of whom are at risk for this kind of cerebral hypoxia), obstructions in the lungs (e.g., hypoxia resulting from choking, strangulation, the crushing of the windpipe).

The term “brain injury biomarker” (BIB), “brain injury biomarker protein”, “brain injury biomarker peptide”, brain injury biomarker polypeptide”, and the like, that can be used in systems and methods according to the principles and exemplary embodiments of the invention, e.g., to diagnose brain injury in a patient. Generally, a BIB is a protein, lipid, nucleic acid (including RNA and DNA), and breakdown product (BDP), modified form, or metabolized form of any of the foregoing biomarkers. For example, BIB proteins include, but are not limited to, biomarkers disclosed herein and in the patents and published patent applications listed below. Accordingly, BIB protein markers include, but are not limited to Aldolase C (ALDOC), Brain derived neurotrophic factor (BDNF), Calcitonin Gene Related Peptide (CGRP), Endothelin 1 (ET1), Eotaxin (CCL11), Fatty Acid Binding Protein 7 (FABP7), Glial Fibrillary Acidic Protein (GFAP), Growth Associated Protein 43 (GAP-43), Intercellular Adhesion Molecule 5 (ICAM-5), Interleukin 6 (IL-6), Interleukin 8 (IL-8), Interleukin 10 (IL-10), Interleukin-33 (IL-33), Metallothionein 3 (MT3), Neurogranin (NRGN), Neurofilament heavy chain (NF-H), Neurofilament light chain (NF-L), Neurofilament medium chain (NF-M), Neuron Specific Enolase (ENO2/NSE), calcium binding protein S100B, Oligodendrocyte Myelin Glycoprotein (OMG), Reticulon (RTN1), Synuclein alpha (SNCA), Synuclein beta (SNCB), Tau microtubule binding protein (TAU/MAPT), von Willebrand Factor (vWF), Vascular Endothelial Growth Factor (VEGF-A, B, C or D homo or heterodimers), brain lipid binding protein (BLBP/FABP7), glutamine synthetase (GS), astrocytic phosphoprotein PEA-15 (PEA15), aB-crystallin (CRYABiHSP27), and all of the protein biomarkers listed in table in FIG. 5.

As stated above, BIB's that can be detected by a multi-analyte assay according to exemplary embodiments of the invention include BDPs, modified forms, and metabolized forms of BIBs. Accordingly, BIB proteins include isoforms and/or post-translationally modified forms of any of the foregoing. For example, modified forms of a BIB protein marker include, but are not limited to, citrullinated, acetylated, methylated, dimethylated, carboxylated, sumoylated, or phosphorylated forms at one or more amino acids of one or more of the protein biomarkers or combinations thereof. Citrullination of BIBs is disclosed in U.S. Patent Application Publication No. 2015/0031048.

Modified forms of protein BIBs also include cleavage products, such as BDPs. Examples of BIBs that are BDPs include, but are not limited to a trauma-specific break down product (BDP) of ALDOC or BLBP/FABP7, trauma-specific proteolytic cleavage product of ALDOC, GS, PEA 15, CRY AB, and a 20-30 kDa BDP of GFAP. In various embodiments, a protein BIB, such as set forth above, is a polypeptide or a fragment thereof having at least about 85% amino acid sequence identity to the amino acid sequence of the specific biomarker protein. In an embodiment, for example, a polypeptide or a fragment thereof has at least about 90%, 95%, or 98% amino acid sequence identity to the amino acid sequence of a specified biomarker protein.

Exemplary embodiments of the invention contemplate the detection, measurement, quantification, determination, and the like of both unmodified and modified (e.g., citrullination or other post-translational modification) proteins/polypeptides/peptides as well as autoantibodies to any of the foregoing. In certain embodiments, it is understood that reference to the detection, measurement, determination, and the like, of a biomarker refers detection of the protein/polypeptide/peptide (modified and/or unmodified). In other embodiments, reference to the detection, measurement, determination, and the like, of a biomarker refers detection of autoantibodies of the protein/polypeptide/peptide.

The term “alteration” or “change” refers to an increase or decrease. An alteration or change may be by as little as 1%, 2%, 3%, 4%, 5%, 10%, 15%, 20%, 25%, 30%, 40%, 45%, 50%, 55%, 60%, or even by as much as 70%, 75%, 80%, 85%, 90%, 95%, or 100%.

As used herein, the terms “comparing”, or “comparison” refers to making an assessment of how the proportion, level or cellular localization of one or more biomarkers in a sample from a patient relates to the proportion, level or cellular localization of the corresponding to one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in standard or control sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels/ratios that correspond to, for example, a patient having brain injury, not having brain injury, is responding to treatment for brain injury, is not responding to treatment for brain injury, is/is not likely to respond to a particular treatment for brain injury, or having/not having another disease or condition. In a specific embodiment, the term “comparing” refers to assessing whether the level of one or more biomarkers in a sample from a patient is the same as, more or less than, different from other otherwise correspond (or not) to levels/ratios of the same biomarkers in a control sample (e.g., predefined levels/ratios that correlate to uninfected individuals, standard brain injury levels/ratios, etc.).

In another embodiment, the terms “comparing”, or “comparison” refers to making an assessment of how the proportion, level or cellular localization of one or more biomarkers in a sample from a patient relates to the proportion, level or cellular localization of another biomarker in the same sample. For example, a ratio of one biomarker to another from the same patient sample can be compared.

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient is improving, not improving, etc. In specific embodiments, the parameter may comprise the level of one or more biomarkers. A particular set or pattern of the amounts of one or more biomarkers may indicate that a patient has improved or worsened.

In other embodiments, a particular set or pattern of the amounts of one or more biomarkers may be correlated to a patient being unaffected (i.e., indicates a patient does not have brain injury). In certain embodiments, “indicating,” or “correlating,” may be by any linear or non-linear method of quantifying the relationship between levels/ratios of biomarkers to a standard, control or comparative value for the assessment of the diagnosis, prediction of brain injury or progression thereof, assessment of efficacy of clinical treatment, identification of a patient that may respond to a particular treatment regime or pharmaceutical agent, monitoring of the progress of treatment, and in the context of a screening assay, for the identification of a therapeutic for brain injury.

The terms “patient,” “individual,” or “subject” are used interchangeably herein, and refer to a mammal, particularly, a human. The patient may have a mild, intermediate, or severe disease or condition. The patient may be an individual in need of treatment or in need of diagnosis based on particular symptoms or personal or family history. In some cases, the terms may refer to treatment in experimental animals, in veterinary application, and in the development of animal models for disease, including, but not limited to, rodents including mice, rats, and hamsters; and primates.

The terms “measuring” and “determining” are used interchangeably throughout and refer to methods which include obtaining or providing a patient sample and/or detecting the level of a biomarker(s) in a sample. In one embodiment, the terms refer to obtaining or providing a patient sample and detecting the level of one or more biomarkers in the sample. In another embodiment, the terms “measuring” and “determining” mean detecting the level of one or more biomarkers in a patient sample. The term “measuring” is also used interchangeably throughout with the term “detecting” or “assessing.” In certain embodiments, the term is also used interchangeably with the term “quantifying.” Where a quantitative and/or qualitative determination is intended, the phrase “determining a level of” or “detecting the level of” a protein, analyte, biomarker, etc. is typically used.

The terms “sample,” “patient sample,” “biological sample,” and the like, encompass a variety of sample types obtained from a patient, individual, or subject and can be used in a diagnostic or monitoring assay. The patient sample may be obtained from a healthy subject or a patient suspected of having or having associated symptoms of brain injury. Moreover, a sample obtained from a patient can be divided and only a portion may be used for diagnosis. Further, the sample, or a portion thereof, can be stored under conditions to maintain sample for later analysis. The definition specifically encompasses blood, cerebrospinal fluid, and other liquid samples of biological origin (including, but not limited to, peripheral blood, serum, plasma, cord blood, amniotic fluid, urine, saliva, stool and synovial fluid), solid tissue samples such as a biopsy specimen or tissue cultures or cells derived therefrom and the progeny thereof. In a specific embodiment, a sample comprises a blood sample. In another embodiment, a sample comprises a plasma sample. In yet another embodiment, a serum sample is used. In certain embodiments, a sample comprises cerebrospinal fluid.

The definition of “sample” also includes samples that have been manipulated in any way after their procurement, such as by centrifugation, filtration, precipitation, dialysis, chromatography, treatment with reagents, washing, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

Various methodologies of the instant invention include a step that involves comparing a value, level, feature, characteristic, property, etc. to a “suitable control,” referred to interchangeably herein as an “appropriate control,” a “control sample,” a “reference” or simply a “control.” A “suitable control,” “appropriate control,” “control sample,” “reference” or a “control” is any control or standard familiar to one of ordinary skill in the art useful for comparison purposes. A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, a “brain injury-positive reference level” of a biomarker means a level of a biomarker that is indicative of brain injury in a subject, and a “brain injury-negative reference level” of a biomarker means a level of a biomarker that is indicative of no brain injury of in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., ELISA, MSD ELISA, PCR, LC-MS, GC-MS, etc.), where the levels of biomarkers may differ based on the specific technique that is used.

In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of may be assayed for levels/ratios in a sample from an unaffected individual (UI) (e.g., no brain injury) or a normal control individual (NC) (both terms are used interchangeably herein). For example, a “suitable control” or “appropriate control” can be a value, level, feature, characteristic, property, ratio, etc. determined prior to performing a therapy (e.g., brain injury treatment) on a patient or a value, level, feature, characteristic, property, ratio, etc. determined prior to disease development (e.g., a baseline test). In yet another embodiment, a protein level/ratio, transcription rate, mRNA level, translation rate, biological activity, cellular characteristic or property, genotype, phenotype, etc., can be determined prior to, during, or after administering a therapy into a cell, organ, or patient. In a further embodiment, a “suitable control” or “appropriate control” is a predefined value, level, feature, characteristic, property, ratio, etc. A “suitable control” can be a profile or pattern of levels/ratios of one or more biomarkers that correlates to brain injury, to which a patient sample can be compared. The patient sample can also be compared to a negative control, i.e., a profile that correlates to not having brain injury.

As used herein, the term “predetermined threshold value of expression” of a biomarker refers to the level of expression of the same biomarker (expressed, for example, in ng/ml) in a corresponding control/normal sample or group of control/normal samples obtained from normal, or healthy, subjects, i.e., subject who do not have brain injury. Further, the term “altered level of expression” of a biomarker in a sample refers to a level that is either below or above the predetermined threshold value of expression for the same biomarker and thus encompasses either high (increased) or low (decreased) expression levels. In particular embodiments, the biomarkers described herein are increased or decreased relative to age-matched controls.

The terms “specifically binds to,” “specific for,” and related grammatical variants refer to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M. As used herein, the terms “specific binding” or “specifically binding” when used in reference to the interaction of an antibody and a protein or peptide means that the interaction is dependent upon the presence of a particular structure (i.e., the epitope) on the protein.

As used herein, the terms “binding agent specific for” or “binding agent that specifically binds” refers to an agent that binds to a biomarker and does not significantly bind to unrelated compounds. Examples of binding agents that can be effectively employed in the disclosed methods include, but are not limited to, proteins and antibodies, such as monoclonal or polyclonal antibodies, or antigen-binding fragments thereof. In certain embodiments, a binding agent binds a biomarker (e.g., a polypeptide biomarker) with an affinity constant of, for example, greater than or equal to about 1×10⁻⁶ M.

By “antibody” is meant any immunoglobulin polypeptide, or fragment thereof, having immunogen binding ability. As used herein, the terms “antibody fragments”, “fragment”, or “fragment thereof” refer to a portion of an intact antibody. Examples of antibody fragments include, but are not limited to, linear antibodies; single-chain antibody molecules and fragments thereof, e.g., scFv; Fc or Fc′ peptides, F(ab) and F(ab′)2 fragments, and multi-specific antibodies formed from antibody fragments, which bind to an antigen. In most embodiments, the terms also refer to fragments that bind an antigen of a target molecule (e.g., a biomarker protein described herein) and can be referred to as “antigen-binding fragments.” As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies that specifically binds the target antigen.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation. An antigenic determinant can compete with the intact antigen (i.e., the “immunogen” used to elicit the immune response) for binding to an antibody.

By “an effective amount” is meant the amount of a required to ameliorate the symptoms of a disease relative to an untreated patient. The effective amount of active compound(s) used to practice exemplary embodiments of the invention for therapeutic treatment of brain injury varies depending upon the manner of administration, the age, body weight, and general health of the subject. Ultimately, the attending physician or veterinarian will decide the appropriate amount and dosage regimen. Such amount is referred to as an “effective” amount.

Ranges provided are understood to be shorthand for all of the values within the range. For example, a range of 1 to 50 is understood to include any number, combination of numbers, or subrange from the group consisting of, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

It is understood that the exemplary embodiments of the invention are not limited to the particular methods and components, etc., described herein, as these may vary. It is also to be understood that the terminology used herein is used for the purpose of describing particular embodiments only, and is not intended to limit the scope of the invention. It is noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth. In addition, for example, reference to “a biomarker” includes reference to more than one biomarker.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the invention.

All publications cited herein are hereby incorporated by reference including all journal articles, books, manuals, published patent applications, and issued patents, including U.S. Pat. Nos. 9,746,481; 9,709,578; 9,709,573, 10,365,288; 10,534,003; 10,557,859; and published patent applications US2018/0024145; US2019/0339291; WO 2018/217792; and WO 2019/099732. In addition, the meaning of certain terms and phrases employed in the specification, examples, and appended claims are provided. The definitions are not meant to be limiting in nature and serve to provide a clearer understanding of certain aspects of the invention.

### BRIEF DESCRIPTION OF THE DRAWINGS

FIG. 1 illustrates and exemplary embodiment of a multimodality system for detection, prognosis, and monitoring of neurological injury and disease constructed in accordance with the principles of the invention.

FIG. 2 illustrates an example of a process flow of using the multimodality system of FIG. 1.

FIG. 3 shows a summary table comparing models developed using covariates to find optimal weightings and example instrument test results used in diagnosis and prognosis determinations using the multimodality system of FIG. 1.

FIG. 4 is a graphical representation of the multimodality system of FIG. 1 showing intermediate outputs of the instruments (modalities).

FIG. 5 is a table listing one or more biomarkers that may be used in accordance with exemplary embodiments of the invention.

FIG. 6 shows sample covariate weightings based on elevated biomarkers in the blood during an injury phase.

FIG. 7 shows sample covariate weightings based on decreased biomarkers in the blood during the injury phase.

FIG. 8 is a table showing differences in blood biomarker levels and standardized neurocognitive test scores between brain injured patients and uninjured control subjects.

FIG. 9 shows correlation of blood biomarkers and standardized neurocognitive test scores in patients with mild TBI.

FIG. 10 shows an expanded correlation of blood biomarkers and functional neurocognitive tests based on multi-modality test panel combinations.

FIG. 11 shows example correlation data in patient outcomes over time.

FIG. 12 shows an example battery of neurocognitive test assessments for use with a system of FIG. 1.

FIG. 13 shows example symptom questionnaires for use with a system of FIG. 1.

FIG. 14 shows an example of process data elements used for determining whether a test subject or patient has TBI.

FIG. 15 shows an example of process data elements to generate a pattern of inputs used for determining whether a test subject or patient has TBI.

FIG. 16 shows an example of process data elements for determining whether a test subject known to have TBI or suspected to have TBI, has an increased likelihood of having a post-injury symptom.

FIG. 17 shows an example of process data elements showing example biomarker detection measurements from an assay reader and neurocognitive and balance assessment scores from a testing application.

FIG. 18 shows an example listing of biomarkers and their characteristics for use in exemplary embodiments of the invention.

FIG. 19A-C show an example sST2 distribution by cohort class.

FIGS. 20A-B show example sST2 distributions by CT status and sex.

FIG. 21 shows an example comparison of models trained to distinguish mild TBI from healthy subjects (HC) showing longitudinal graphs of sST2 distributions measured at varying timepoints after TBI.

FIG. 22 shows example correlations between acute sST2 levels and other identified biomarkers.

FIG. 23 shows another example comparison of models trained to distinguish mild TBI from healthy subjects (HC) showing longitudinal graphs of sST2 distributions measured at varying timepoints after TBI

FIGS. 24-24D show example models trained to distinguish TBI from a group of control subjects that includes both healthy controls and orthopedic injury, with no TBI (CONTROL).

FIG. 25 shows a univariate analysis from Example 9 for the groups studied.

### DETAILED DESCRIPTION

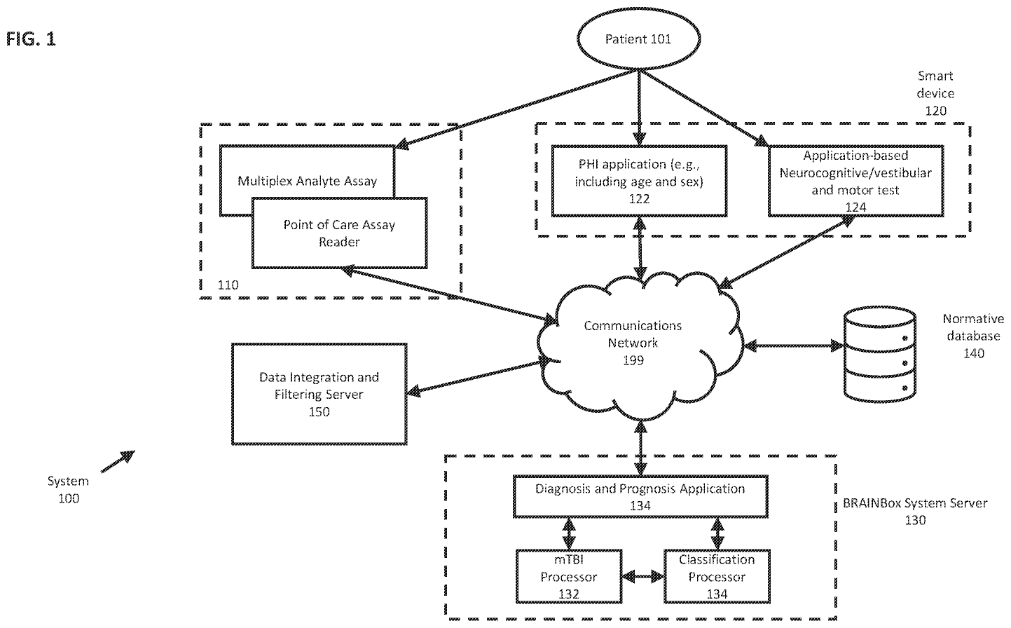

Exemplary embodiments of the invention feature systems and methods for detection, prognosis, and monitoring of neurological injury and disease. FIG. 1 shows a block diagram of a multimodality system 100 for detection, prognosis, and monitoring of neurological injury and disease in which illustrative embodiments of the invention can be implemented. System 100 includes communications network 199. Network 199 is the medium used to provide communications links between various devices and computers connected together within the system 100. Network 199 can include connections, such as wire, wireless communication links, or fiber optic cables. Network 199 can represent a collection of networks and gateways that use the Transmission Control Protocol/Internet Protocol (TCP/IP) and other communication protocols to communicate with one another and with devices connected to the network 199. One example communication network 199 is the Internet, which can include data communication links between major nodes and/or host computers, including thousands of commercial, governmental, educational, and other computer systems that route data and messages. System 100 can also be implemented over a number of different types of networks, such as for example, an intranet, a local area network (LAN), or a wide area network (WAN). FIG. 1 is one example of an environment of the invention and is not an architectural limitation for different illustrative embodiments of the invention.

Clients and servers are only example roles of certain data processing systems and computer systems connected to network 199 do not exclude other configurations or roles for these data processing systems. BRAINBox server 130 and data integration and filtering server 150 connect to network 199 along with storage unit (normative database) 140. Software applications can execute on any computer in the system 100. User computers (clients), including point of care assay reader 110 and smart device 120, are also connected to network 199. A data processing (computer) system, such as servers 130, 150 and clients 110, 120 (and databases, such as normative database 140) can include data and can have software applications and/or software tools executing on them.

FIG. 1 shows an example system architecture and shows certain components that are usable in an exemplary implementation of the invention. For example, servers 130, 150 and clients 110, 120 are depicted as servers and clients only as example and not to imply a limitation to a client-server architecture. In another example embodiment of the invention, the system 100 can be distributed across several data processing (computer) systems and a data network as shown. Similarly, in another example embodiment of the invention, the system 100 can be implemented on a single data processing system within the scope of the illustrative embodiments. Data processing (computer) systems 110, 120, 130, 150 (and databases, such as normative database 140, for example) also represent example nodes in a cluster, partitions, and other configurations suitable for implementing an embodiment of the invention.

The computers (e.g., 110, 120, 130, 150) can take the form of a smartphone, a tablet computer, a laptop computer, a desktop computer, a wearable computing device, or any other suitable computing device and can be servers, personal computers, and/or network computers. Software application programs described as executing in the system 100 in FIG. 1 can be configured to execute in user computers in a similar manner. Data and information stored or produced in another data processing system can be configured to be stored or produced in a similar manner.

Applications 122, 124 implement an embodiment or function of the invention as described in this document. For example, PHI application 122 receives an entry of patient information, including Protected Health Information (PHI) that can include profile elements, demographic information, and/or other personal health information. Application 122 implements an embodiment or a function as described to operate in conjunction with application 134 on the BRAINBox server 130. For example, application 122 produces patient health information used by diagnosis and prognosis application 134 to process, classify, and provide actionable diagnosis and treatment recommendations. Similarly, neurocognitive/vestibular and motor test application 124 operates in conjunction with application 134 on the BRAINBox server 130 and provides neurocognitive/vestibular and motor test results used by diagnosis and prognosis application 134 to process, classify, and provide actionable diagnosis and treatment recommendations.

Computers 110, 120, 130, 150, and storage units (database 140), and additional computers (e.g., clients and servers) may couple to network 199 using wired connections, wireless communication protocols, or other suitable data connectivity.

In the depicted example, BRAINBox server 130 may provide data, such as boot files, operating system images, and applications to user computers (clients) 110, 120. Clients 110, 120 may be clients to server 130 in this example. Clients 110, 120 or some combination, may include their own data, boot files, operating system images, and applications. System 100 may include additional servers, clients, and other devices that are not shown. For example, while countless point of care assay readers, data integration and filtering servers, and smart devices can be used to provide inputs to the BRAINBox server using systems and methods constructed according to the principles and exemplary embodiments of the invention, for clarity and brevity, a single patient A is shown with a single point of care assay reader, a single smart device, and a single data integration and filtering server as shown in FIG. 1.

Among other uses, system 100 may be used for implementing a client-server environment in accordance with exemplary embodiments of the invention. A client-server environment enables software applications and data to be distributed across a network such that an application functions by using the interactivity between a user computer and a server. System 100 may also employ a service-oriented architecture, where interoperable software components distributed across a network can be packaged together as coherent applications.

Together, the system 100 provides inputs for the diagnosis and prognosis application 134 to process, classify, and provide actionable diagnosis and treatment recommendations. More specifically, a multiplex fluorescence immunoassay (used in point of care assay reader 110) uses a panel of in vitro diagnostic blood measurements of a combination of biomarkers providing a single result derived by an AI-driven process that includes two or more of the following: Neurogranin (NRGN), Neuron Specific Enolase (ENO2/NSE), Metallothionein 3 (MT3), (Intercellular Adhesion Molecule 5 (ICAM-5), Synuclein Beta (SNCB), Interleukin 6 (IL-6), Neurofilament light chain (NF-L), Tau microtubule binding protein (TAU/MAPT), Aldolase C (ALDOC), Fatty Acid Binding Protein 7 (FABP7), Oligodendrocyte Myelin Glycoprotein (OMG), von Willebrand Factor (vWF), Vascular Endothelial Growth Factor (VEGF), and/or Glial Fibrillary Acidic Protein (GFAP). The system 100 uses inputs from PHI application 122, including age and sex of the patient, for example, as further inputs for the diagnosis and prognosis application 134. Additionally, application-based neurocognitive/vestibular and motor tests 124 can include test results for balance, cognitive processing speed assessed in immediate and delayed recall tasks, and cognitive acuity tasks. The results of the neurocognitive/vestibular and motor tests 124 are used as further inputs to the diagnosis and prognosis application 134 for processing, classification, and recommendations for actionable diagnosis and prognosis determinations.

### Multiplex Analyte Assay and Point of Care Assay Reader

An early and accurate diagnosis of a TBI or any disease plays a decisive role for its effective treatment. Especially at the point of care, where an immediate decision on treatment most needs to be made, the rapid and precise confirmation of clinical findings is vital.

The point of care readers 110 constructed in accordance with exemplary embodiments of the invention incorporate low sample consumption (e.g., blood from a finger prick), or other easily accessible (noninvasive) samples, like urine, saliva, sweat and breath condensate, for example. The point of care readers 110 provide simple or automated system operation requiring minimal user intervention and provide rapid turnaround times (e.g., 10 minutes to 2 hours) that allow an immediate treatment. The point of care readers 110 have a prolonged reagent storage and shelf life while providing accurate and quantitative results in accordance with clinical and central laboratory findings, adhering to international quality standards (ISO 15189). The point of care readers constructed in accordance with exemplary embodiments of the invention are low-cost and portable readout devices, equipped with disposable test cartridges or strips, satisfying in vitro diagnostics guidelines (EU Directive 98/79/EC or FDA regulations).

In one example embodiment of the invention, a multiple analyte assay, such as a microarray substrate with customized printing of capture antibodies for multiple analytes is submitted to the point of care reader 110. Point of care reader 110 provides simultaneous on-site detection of different analytes from a single specimen. Different multiplexing technologies (e.g., bead- or array-based systems) can be used in the point of care reader 110 along with their detection methods (e.g., electrochemical or optical).

Biomarkers of several types have been developed to detect neurological injury in individuals suspected of or witnessed as having a head injury. Biofluid detection of brain injury proteins (BIBs) released by cellular damage has included mostly blood testing, since blood is easily accessible and routinely drawn in an emergency department setting as standard of care. However, biofluid samples collected from cerebrospinal fluid (CSF), nasal fluid, a saliva, a urine, a sputum, a secretion (e.g. tears), sweat, and organ tissue are also sources for biomarkers.

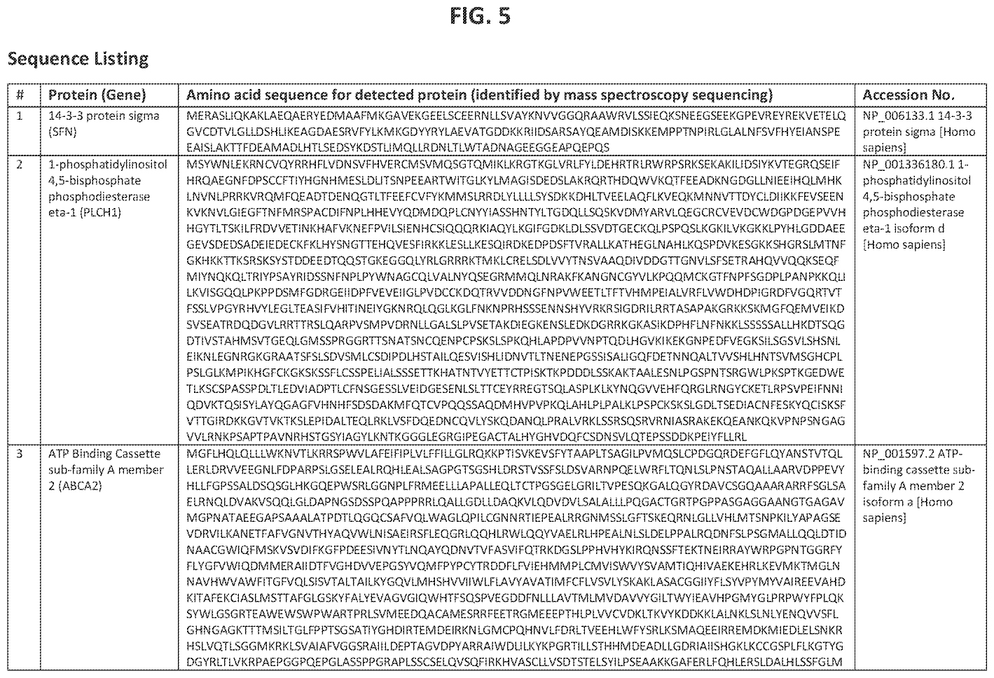

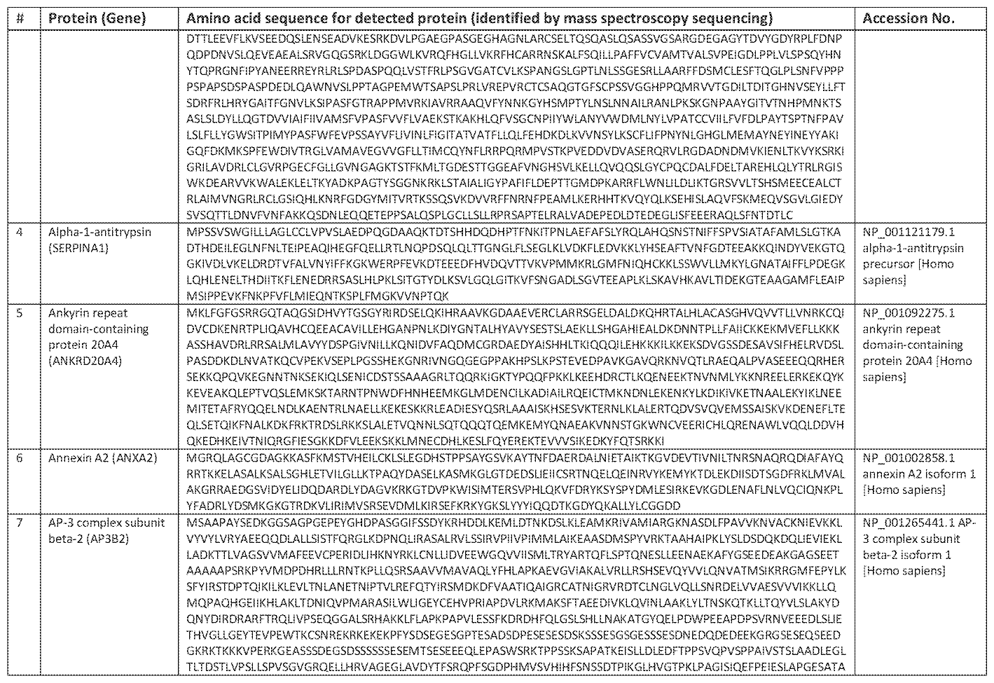

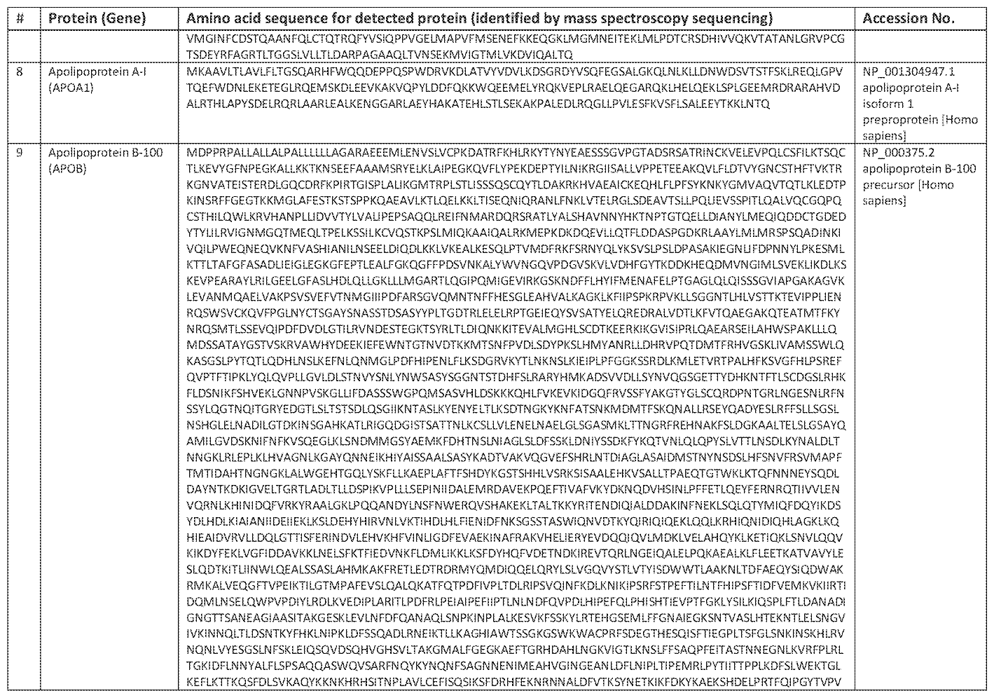

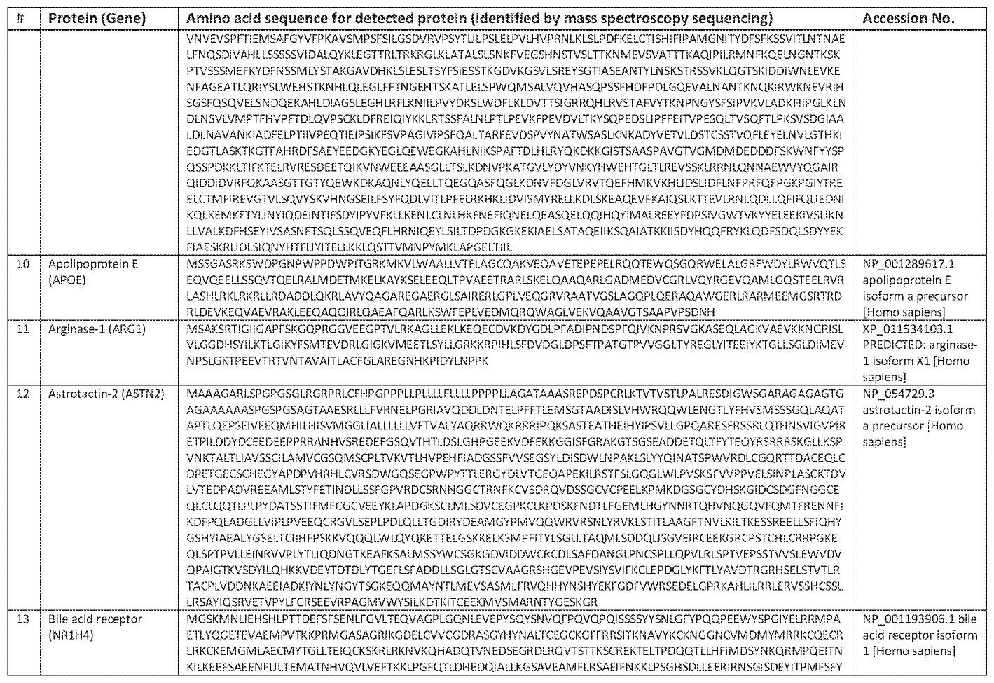

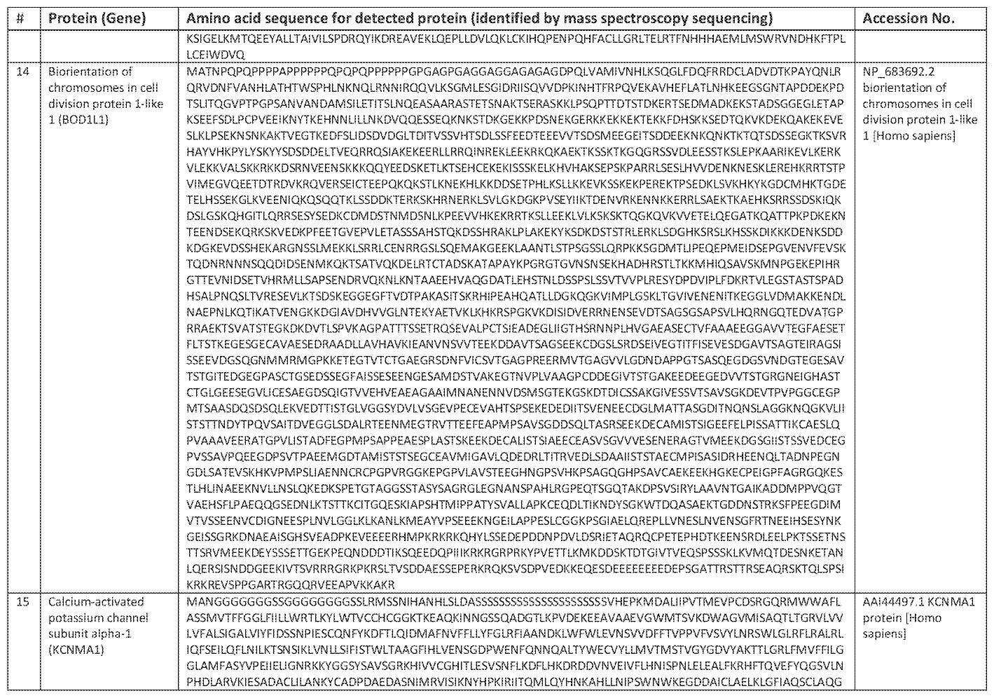

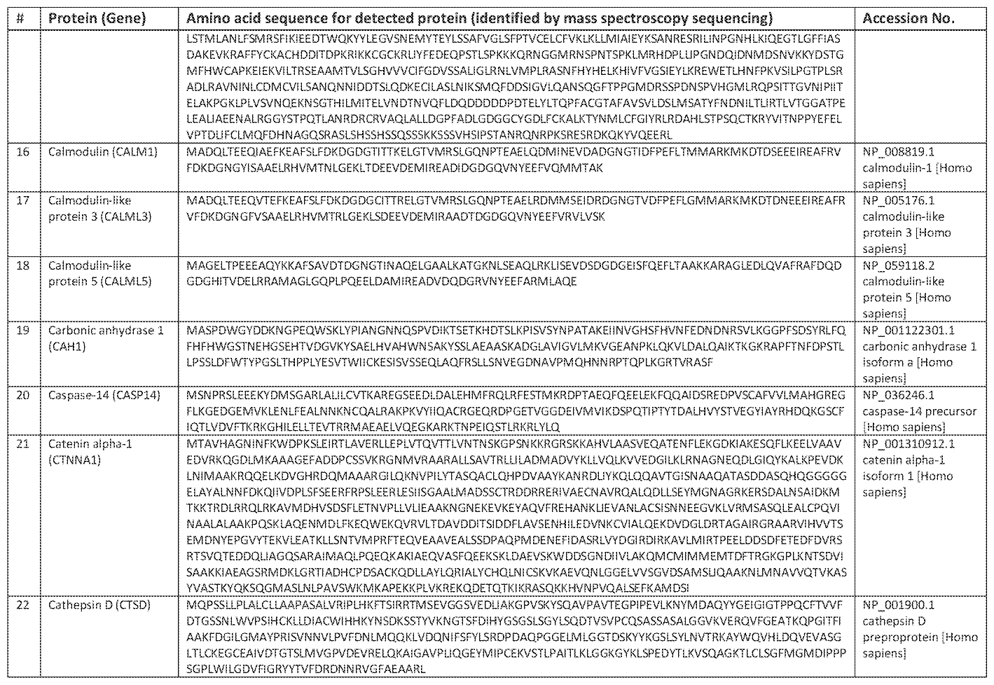

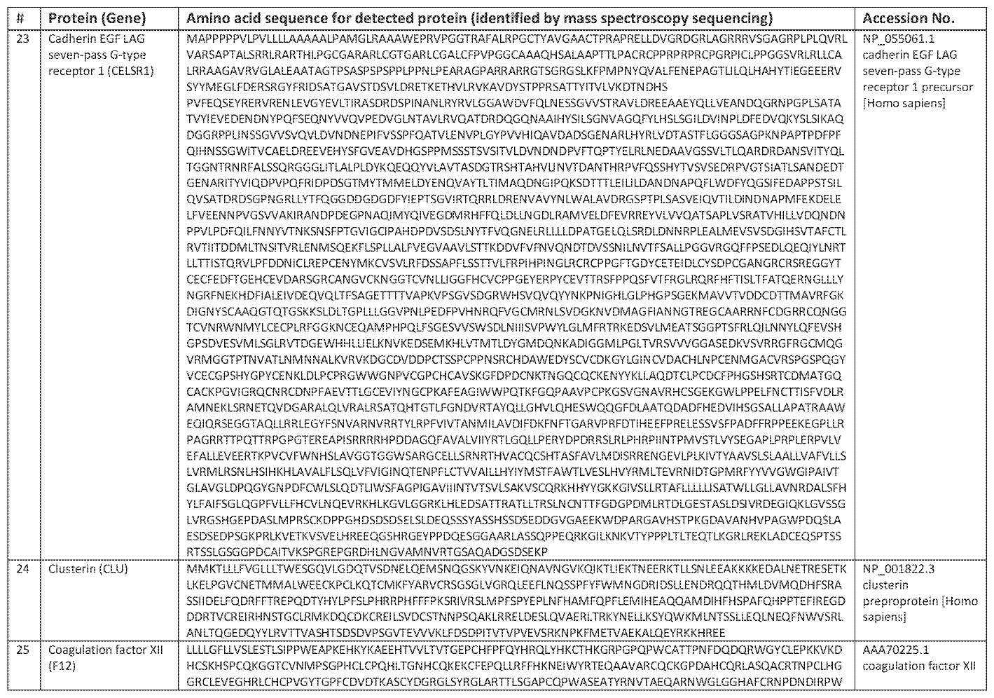

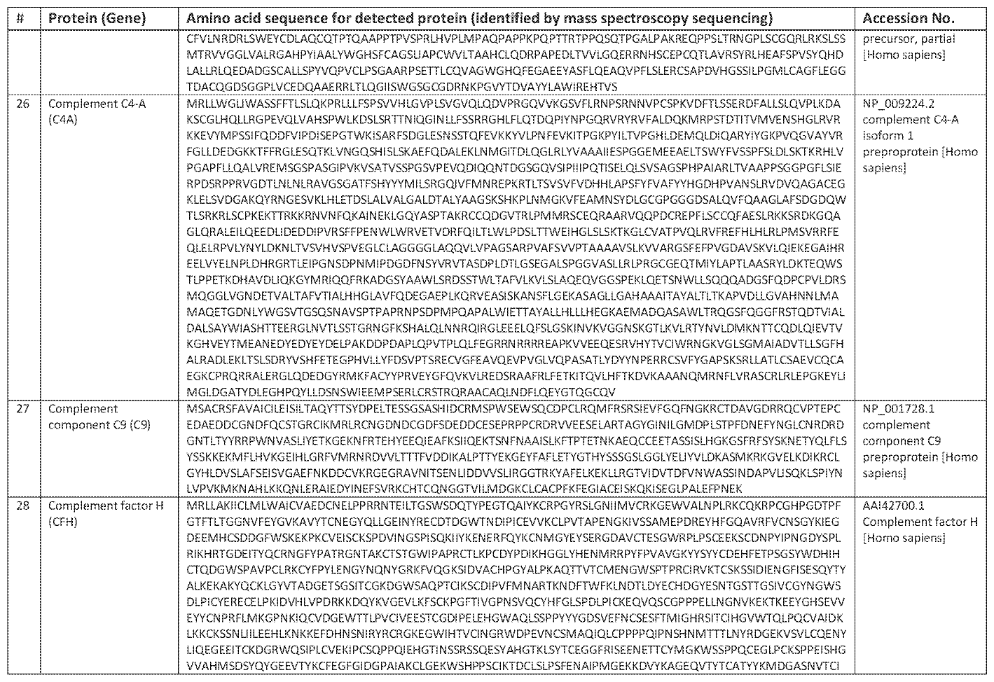

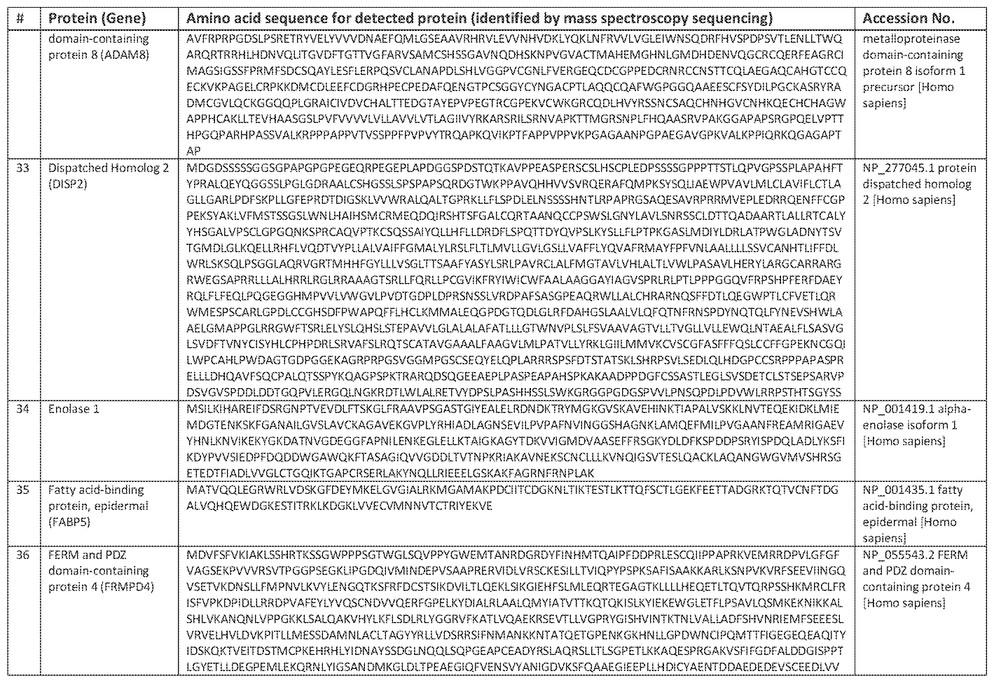

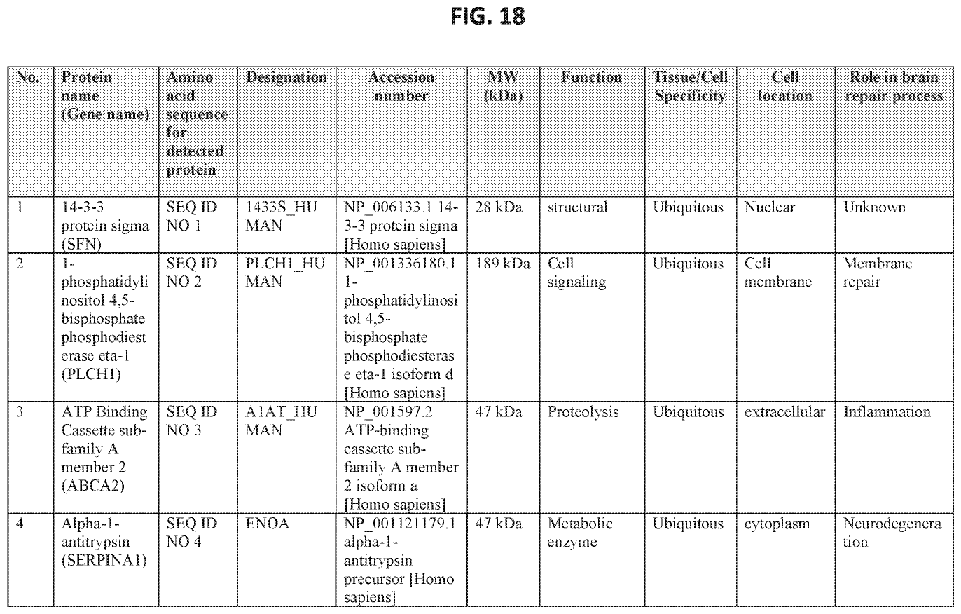

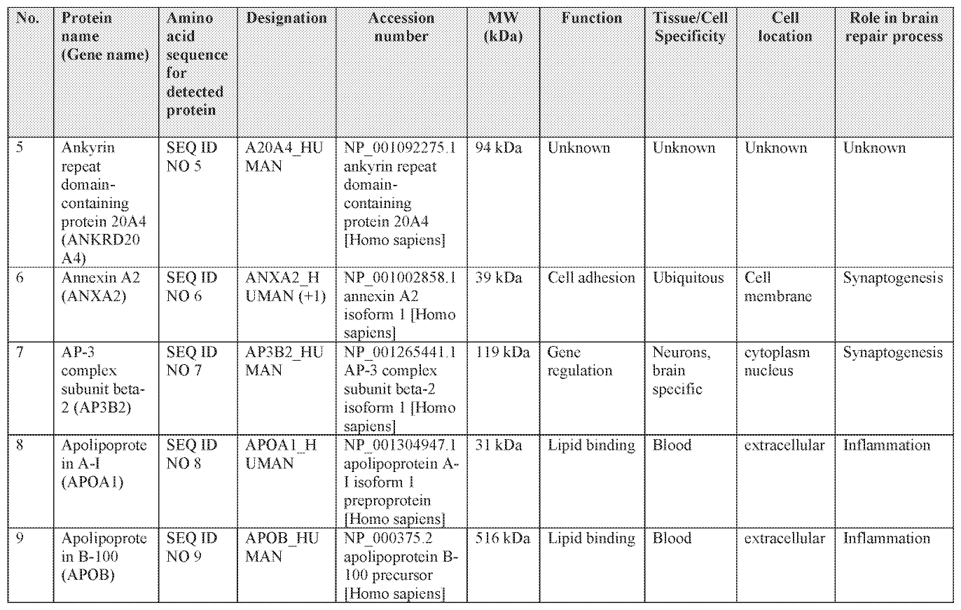

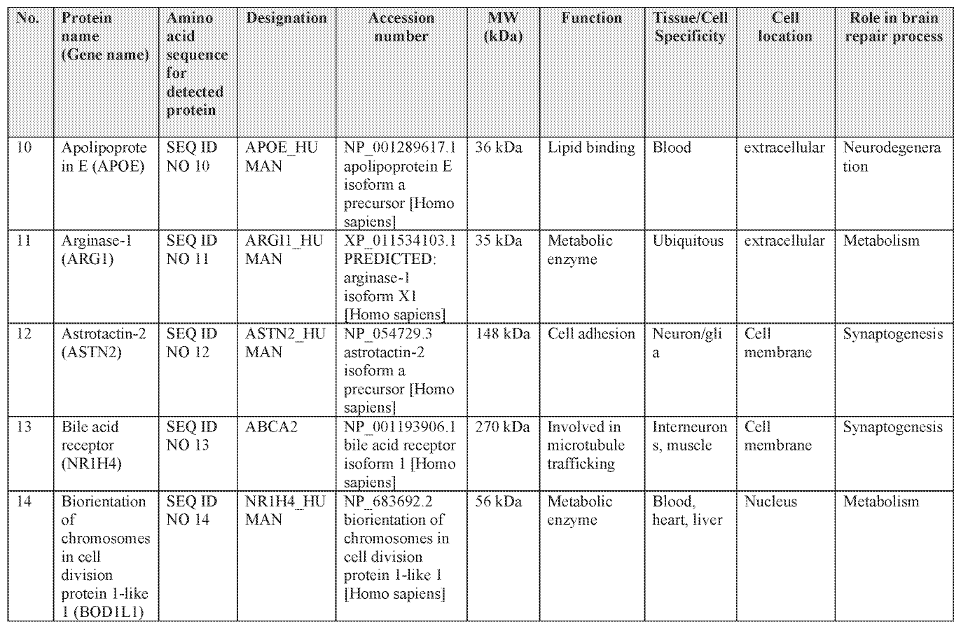

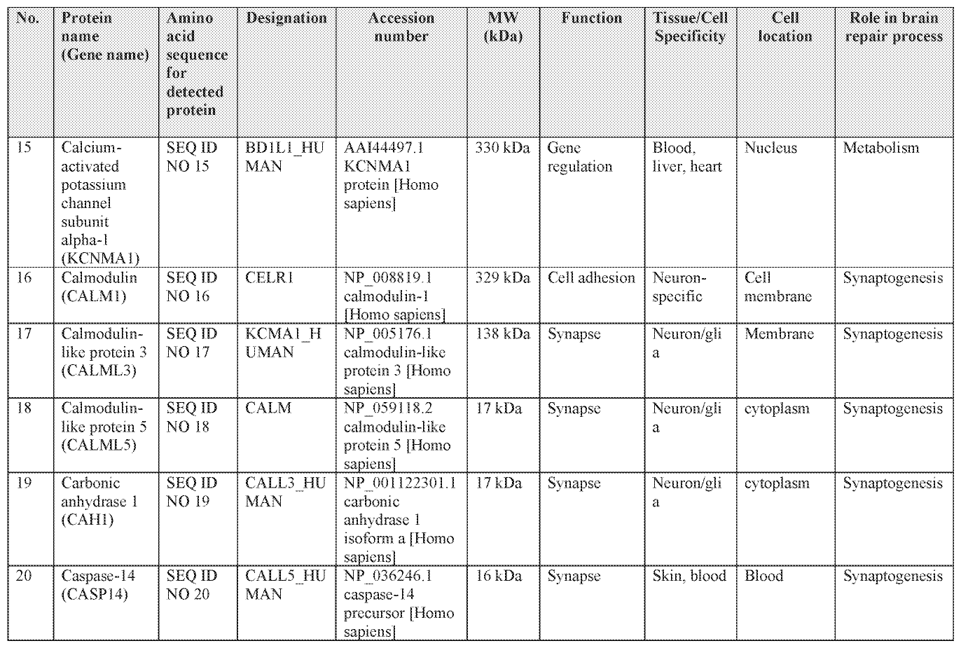

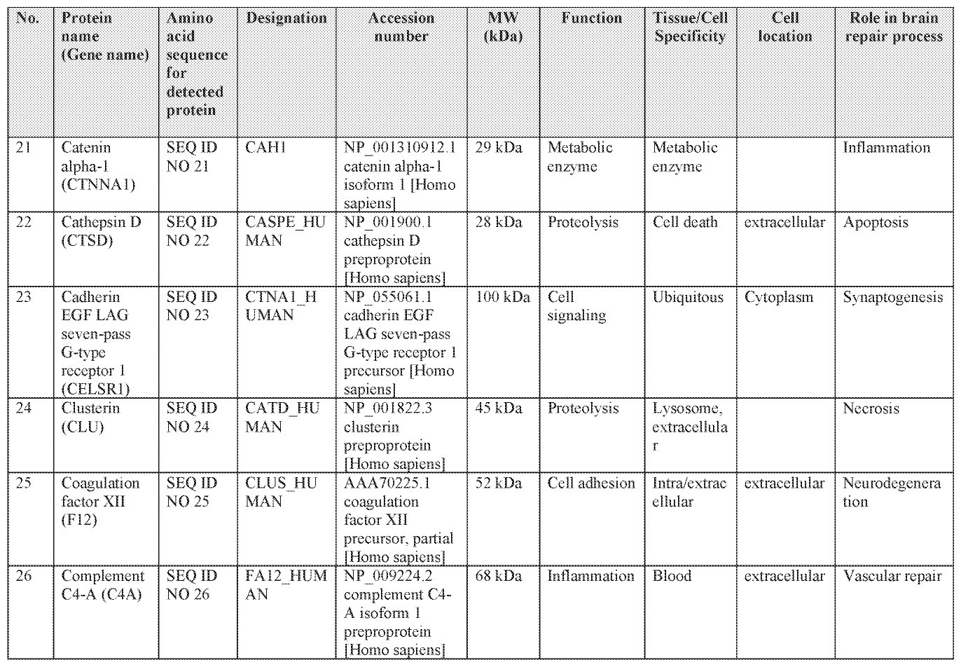

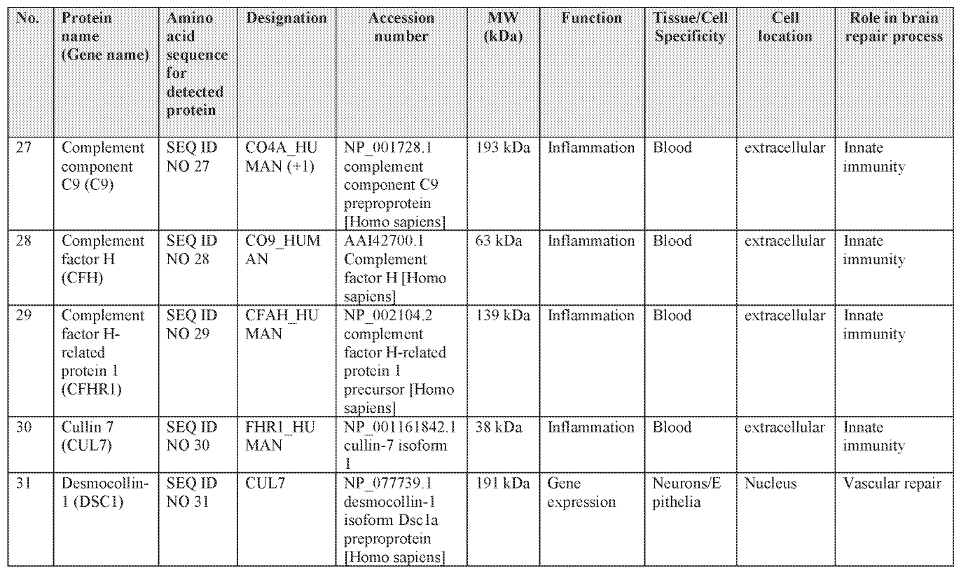

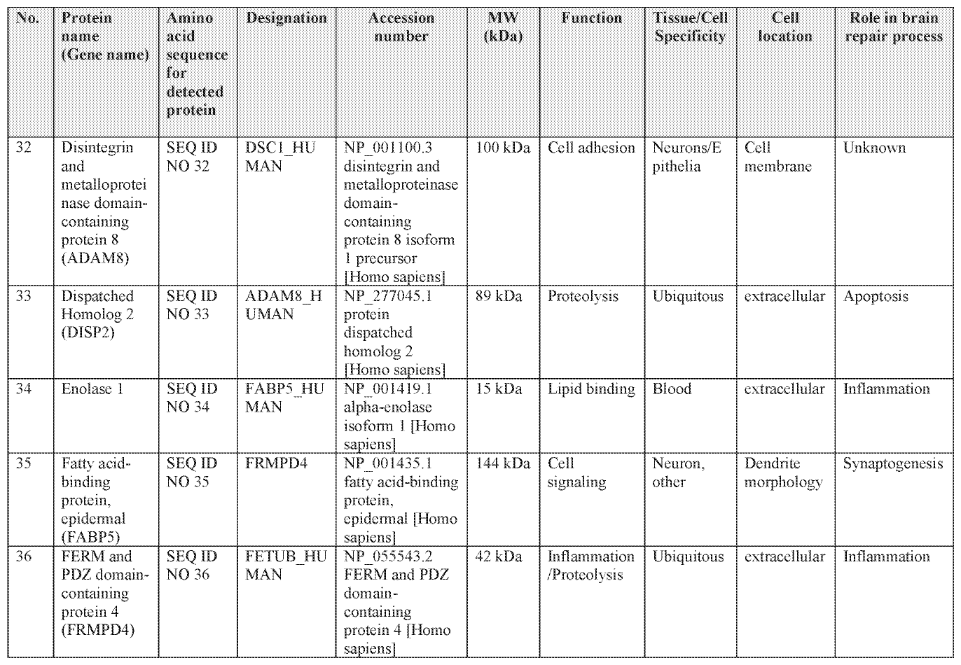

In various embodiments, the biomarkers detected, or detected and quantified, are at least one of proteins, lipids, nucleic acids (including RNA and DNA), and breakdown products (BDP), modified forms, or metabolized forms of any of proteins, lipids, nucleic acids. Accordingly, in some embodiments, BIB proteins include, but are not limited to, biomarkers disclosed herein and in the patents and published applications incorporated by reference and described in this application. Accordingly, BIB protein markers include, but are not limited to Aldolase C (ALDOC), Brain derived neurotrophic factor (BDNF), Calcitonin Gene Related Peptide (CGRP), Endothelin 1 (ET1), Eotaxin (CCL11), Fatty Acid Binding Protein 7 (FABP7), Glial Fibrillary Acidic Protein (GFAP), Growth Associated Protein 43 (GAP-43), Intercellular Adhesion Molecule 5 (ICAM-5), Interleukin 6 (IL-6), Interleukin 8 (IL-8), Interleukin 10 (IL-10), Interleukin-33 (IL-33), Metallothionein 3 (MT3), Neurogranin (NRGN), Neurofilament heavy chain (NF-H), Neurofilament light chain (NF-L), Neurofilament medium chain (NF-M), Neuron Specific Enolase (ENO2/NSE), calcium binding protein S100B, Oligodendrocyte Myelin Glycoprotein (OMG), Reticulon (RTN1), Synuclein alpha (SNCA), Synuclein beta (SNCB), Tau microtubule binding protein (TAU/MAPT), von Willebrand Factor (vWF), Vascular Endothelial Growth Factor (VEGF-A, B, C or D homo or heterodimers), brain lipid binding protein (BLBP/FABP7), glutamine synthetase (GS), astrocytic phosphoprotein PEA-15 (PEA15), aB-crystallin (CRYABiHSP27), and all of the protein biomarkers listed in the table shown in FIG. 18 and in the sequence listing table shown in FIG. 5).

Accordingly, in certain embodiments, a system constructed according to the principles of the invention incudes a multi-analyte assay capable of detecting, or detecting and quantitating, at least one of the following subsets of BIB proteins listed in the tables shown in FIGS. 5 and 18: (i) a cell adhesion protein, a cell signaling protein, a cell toxicity protein, a clotting protein, a cytoskeleton protein, an extracellular matrix protein, a gene expression mediating protein, a gene regulation protein, an inflammation protein, a microtubule trafficking protein, a lipid binding protein, a metabolic enzyme, a metabolism protein, a protein-binding protein, a proteolytic protein, a signaling protein, a structural protein, a synapse protein; (ii) at least one protein biomarker found in mammalian cells or tissue, selected from a protein found in astrocytes, one or more proteins found in blood, one or more protein found in blood, heart and liver tissue, one or more proteins found in brain tissue, a protein found in cardiac tissue, a protein found in epithelial tissue, a protein found in interneurons, a protein found in neuroepithelial cells, one or more proteins found in neurons, a protein found in skin tissue, one or more ubiquitous proteins, and combinations thereof; (iii) a protein with a role in a brain repair process selected from one or more apoptosis proteins, one or more inflammation proteins, one or more innate immunity proteins, one or more membrane repair proteins, one or more metabolism proteins, one or more necrosis proteins, one or more neurodegeneration proteins, one or more neurogenesis proteins, one or more synaptogenesis proteins, one or more vascular repair proteins; or (iv) combinations of (i), (ii), and (iii).

In one advantageous embodiment, a system according to the invention incudes a multi-analyte assay capable of detecting, or detecting and quantitating, a subset of biomarkers comprising at least one of: Protein No.12, Astrotactin 2 (ASTN2); Protein No. 30, Cullin-7 (CUL7); Protein No. 50, metallothionein 1 isoform X (MT1X); Protein No. 67, Slit-Robo GTPase protein (SRGAP1); and Protein No. 79, von Willebrand Factor (vWF). In another embodiment, the multi-analyte assay detects, or detects and quantitates Protein No.12, Astrotactin 2 (ASTN2); Protein No. 30, Cullin-7 (CUL7); Protein No. 50, metallothionein 1 isoform X (MT1X); Protein No. 67, Slit-Robo GTPase protein (SRGAP1); and Protein No. 79, von Willebrand Factor (vWF), and at least one BIB in the subset of BIBs Brain-Derived Neurotrophic Factor (BDNF); Glial Fibrillary Acidic Protein (GFAP); Intracellular Adhesion Molecule 5 (ICAM5); Synuclein Beta (SNCB); Metallothionein 3 (MT3); Neurogranin (NRGN); Neuron Specific Enolase (NSE); and Aldolase C (ALDOC).

As stated above, BIBs that can be detected by a multi-analyte assay constructed according to exemplary embodiments of the invention include BDPs, modified forms, and metabolized forms of BIBs. Accordingly, BIB proteins include isoforms and/or post-translationally modified forms of any of the foregoing. For example, a modified forms of a BIB protein marker include, but are not limited to, citrullinated, acetylated, methylated, dimethylated, carboxylated, sumoylated, or phosphorylated forms at one or more amino acids of one or more of the protein biomarkers or combinations thereof. Citrullination of BIBs is disclosed in U.S. Patent Application Publication No. 2015/0031048.

Modified forms of protein BIBs also include cleavage products, such as BDPs. Examples of BIBs that are BDPs include, but are not limited to a trauma-specific break down product (BDP) of ALDOC or BLBP/FABP7, trauma-specific proteolytic cleavage product of ALDOC, GS, PEA 15, CRY AB, and a 20-30 kDa BDP of GFAP. In various embodiments, a protein BIB, such as described above, is a polypeptide or a fragment thereof having at least about 85% amino acid sequence identity to the amino acid sequence of the specific biomarker protein. In an embodiment, for example, a polypeptide or a fragment thereof has at least about 90%, 95%, or 98% amino acid sequence identity to the amino acid sequence of a specified biomarker protein.

Exemplary embodiments of the invention contemplate the detection, measurement, quantification, determination, and the like of both unmodified and modified (e.g., citrullination or other post-translational modification) proteins/polypeptides/peptides as well as autoantibodies to any of the foregoing. In certain embodiments, it is understood that reference to the detection, measurement, determination, and the like, of a biomarker refers to detection of the protein/polypeptide/peptide (modified and/or unmodified). In other embodiments, reference to the detection, measurement, determination, and the like, of a biomarker refers detection of autoantibodies of the protein/polypeptide/peptide.

As stated above, in addition to proteins, biomarkers according to the invention also include lipids and nucleic acids. For example, in certain embodiments, a system according to the invention includes a multi-analyte assay for detecting the presence and/or levels of one or more of the following lipids used as biomarkers of brain injury: diglyceride; triglyceride; cholesterol ester; phosphatidylcholine; lysophosphotidylcholine; phosphatidylethanolamine; lysophosphatidylethanolamine; sphingomyelin; ceramide; hexosylceramide; phosphatidylserine; and polyunsaturated fatty acid (PUFA). In other embodiments, a system according to the invention includes a multi-analyte assay for detecting the presence and/or levels of one or more of the following nucleic acids used as biomarkers of brain injury: microsatellite DNA; genomic DNA and genomic DNA fragments; tRNA; mRNA long coding nonsense RNAs (IcnRNAs); and microRNAs miR-let7a; miR-7; miR-10a; miR-10b; miR16; miR-21; miR-26a; miR27b; miR29a; miR-30b; miR-107; miR-128; miR-130a; miR-133a-5p; miR 151a; miR181a; miR221; miR-223; miR-292-5p; miR-320; miR-433-3p; miR-451; miR532; miR-541; miR629; miR-711; miR 769; and miR-1307.

The point of care readers 110 constructed in accordance with exemplary embodiments of the invention provide accurate, reproducible biomarker results that can be received by BRAINBox System Server 130 and further examined for use in diagnosis and prognosis application 134.

### Digital Neurocognitive and Motor Testing Device.

In addition to biomarker results from point of care readers 110, digital and neurocognitive and motor test results are also provided to BRAINBox server 130. Digital neurocognitive and motor testing may be integrated on a hand-held device, such as smart device 120 shown in FIG. 1. Other smart phones, or other portable (personal) computers can be configured to run as smart device 120. The device provides a rapid, mobile, and user-friendly cognitive assessment of the patients. The apps in accordance with exemplary embodiments of the invention provide digital testing of cognitive processing speed, immediate and delayed recall, depressive symptoms, anxiety, and trauma related stress.

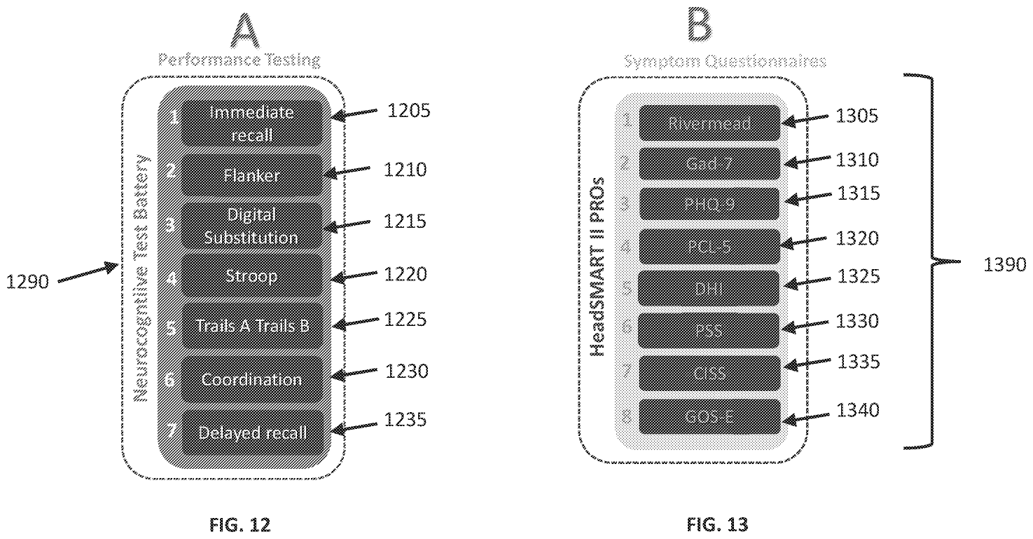

An example of available neurocognitive test assessments is shown in the example Neurocognitive Test Batter Area A of FIG. 12. Performance testing is carried out using one or more of neurocognitive assessments including Immediate recall 1205, Flanker 1210, Digital Substitution 1215, Stroop 1220, Trials A Trials B 1225, Coordination 1230, and Delayed 1235. The neurocognitive assessments 1205, 1210, 1215, 1220, 1225, 1230, 1235 form a neurocognitive test battery 1290 and provide more information about a patient's cognitive capabilities than a basic neurological examination. The battery of tests, in concert with the biomarker information, help determine areas of the brain that are impacted by a TBI and how these impairments impact day-to-day functioning. Likewise, the BRAINBox server 130 uses the test results to help determine how other factors (e.g., depression, anxiety, stress, etc.) are impacting cognitive functioning to form a diagnosis, a prognosis, and a treatment plan. User input for each neurocognitive performance test is captured from the smart device 120, aggregated, and normalized to generate a standardized score for each assessment 1205, 1210, 1215, 1220, 1225, 1230, 1235 and for the battery 1290. The standard composite score is calculated by comparing the raw composite score to our normative data for those in the same age range taking the test on the same type of device, and scaled to have 100 as the mean and 15 as the standard deviation. Any value is smaller than 0 or bigger than 200 are set to 0 and 200, respectively The value of the standard composite score is shown directly on the clinical report and also displayed as a black dot on the bell curve. For both composite score and the assessment scores, their percentile rank within the population was derived from our normative database.

In addition to the neurocognitive test assessments, patient symptoms are collected at the point (time) of injury and at intervals after the injury. FIG. 13 shows example symptom questionnaires that can be used at the point of injury and to record patient outcomes at follow-up visits, such as at the 14-day point, at the one-month mark, and at the three-month mark. An electronic data capture device (such as smart device 120 for one example) can be used to select an appropriate symptom questionnaire 1390, including Rivermead Post-Concussive Symptom Questionnaire (RPQ-16) 1305, Generalized Anxiety Disorder Questionnaire 7 questions (GAD-7) 1310, Patient Health questionnaire-9 questions (PHQ-9) 1315, PTSD Checklist for DSM-5 (PCL-5) 1320, Dizziness and Headache Inventory (DHI) 1325, Perceived Stress Scale (PSS) 1330, Convergence Insufficiency Symptom Survey (CISS) 1335, Montreal Cognitive Assessment (MoCA), Mini Mental State Exam (MMSE), Saint Louis University Mental Status Examination (SLUMS), Hopkins Verbal Learning Test-Revised (HVLT-R), and/or Glasgow Outcome Score-extended (GOS-E) 1340. The symptom questionnaires are used to measure the severity of post-concussive symptoms of patients suspected of suffering a Traumatic Brain Injury (TBI). The results of the symptom questionnaires are received from a user (e.g., via smart device 120), aggregated, and normalized to generate a standardized score that is received by the BRAINBox server 130. The standardized score of the symptom questionnaires is used at the point of injury as well as at the follow-up visits to track symptoms and the recovery process, as well as for use in further correlating biomarker measurements, neurocognitive test assessments, patient symptoms, and outcomes.

The digital and neurocognitive and motor testing apps are modular and can be updated and upgraded to provide additional testing results to the BRAINBox server 130. Additional digital and neurocognitive and motor testing apps can be substituted into the process to provide new and different inputs to the diagnosis and prognosis application 134 in the BRAINBox server 130.

Use of oculomotor function to assess brain functional deficits has also been used. Vestibular-Ocular Motor (VOM) dysfunction is common after TBI. These systems are assessed by testing balance (vestibular component) or the way the eyes track a visual cue or stimulus, and test the smoothness of the oculomotor coordination and the ability to effectively accommodate while focusing on an object at changing distances. “Eye-tracking” devices take into account the differences in function (deficits) that occur in the VOM systems of the brain of injured subjects and have been proposed for diagnosis of brain injury. Different application-based neurocognitive/vestibular and motor tests can be used in accordance with exemplary embodiments of the invention.

Additionally, EEG signals have been used to diagnose TBI using a flexible array of dry electrodes. Flexible and portable EEG arrays can provide rapid, objective assessments of the likelihood of TBI in patients who present with mild symptoms at the point of care, which could be outside the hospital setting in a field-based medical application, in an urgent care clinic, or in a standard emergency room setting.

Non-biofluid biomarkers of TBI have included mainly neuroimaging biomarkers derived from computed tomography (CT) scans or magnetic resonance imaging (MRI), but have also been developed from electroencephalogram (EEG) wave patterns and signal properties. Where neuroimaging features themselves are diagnostic, prognosis has also been described using data from MRI. These include measures of fluid accumulation and structural features of nerve fiber tracts that differ from normative control databases or pre-injury scans from the same subject.

In addition to these example neurocognitive test assessments, including oculomotor function tests, EEGs, and non-biofluidic biomarkers, as new and different (neurocognitive/vestibular and motor) tests are developed, they can be tested and collected using smart device 120 and associated application 124 to provide new and different inputs to the diagnosis and prognosis application 134 in the BRAINBox server 130 for use in generating a diagnostic score and prognostic risk scores.

### BRAINBox Server

The BRAINBox server 130 integrates elements and testing results from the PHI application 122, application-based neurocognitive/vestibular and motor test 124 and point of care reader 110 and generates a diagnostic score and prognostic risk scores for post-acute TBI symptom categories as measures of likelihood of patient outcomes.

The diagnostic and prognosis application 134 incorporates machine learning algorithms (MLAs) that use cloud-based AI data analytics to automatically generate a TBI diagnostic call score and an accompanying prognosis report, reflecting the severity of each patient's TBI and a clinical prognosis. The MLAs use and refine subsets of biomarkers with different weightings as well as the results of the digital cognitive testing assessments and PHI application results to classify patients and facilitate the understanding of brain injury status, as well as patient classification and risk profiles.

According to some embodiments of the invention, the generated diagnostic score and prognostic risk scores can provide clinically useful information relevant to traumatic brain injury (TBI). For example, they can discriminate between subjects with TBI and those without TBI. In some cases, subjects with TBI, but with no intracranial hemorrhage, may have a concussion. In such cases, differences in the diagnostic scores and prognostic risk scores relative to a control subject may be used to identify those patients with a concussion or a significant concussion. The described methods have broad applicability not only in diagnosing athletes and those who play sports, but others as well. Blood or serum biomarker levels are used to determine whether the athlete or sports player has had significant brain injury, such as a concussion (may not return to play immediately) or has not had a significant brain injury or a concussion (may return to play immediately), but also in determining whether an individual can return to work.

For athletes, sports players, military personnel, and other subjects suspected of sustaining mild TBI, the current diagnostic paradigm is frequently based on subjective patient reports of symptoms and physical exam findings. As a result, there is an unmet clinical need for a diagnostic test that can objectively discriminate TBI among undifferentiated blunt head injury patients. The BRAINBox servers 130 in accordance with the invention integrates biomarkers from point of care reader 110 with testing results from the PHI application 122, and application-based neurocognitive/vestibular and motor tests 124 to determine a diagnostic score and prognostic risk scores for post-acute TBI symptom categories.

### Diagnosis and Prognosis Application

Diagnosis and prognosis application 134 processes, classifies, and recommends actionable diagnosis and prognosis determinations. In one example embodiment of the invention, diagnosis and prognosis application 134 is part of BRAINBox server 130, but in other example embodiments, diagnosis and prognosis apps 134 can be run from separate servers or other computers.

In diagnosis and prognosis application 134, several statistical modeling methods are used to distinguish between patient populations or sub-populations (subclasses, clinically different), such as to distinguish between the adjudicated TBI positive (TBI+) vs TBI negative (TBI−), including multivariable logistic regression, random forest, support vector machines, gradient boosting, and Bayesian methods. According to Wolpert's no free lunch theorem, no method performs optimally for every modeling scenario. Therefore, different methods are used to identify a best performing model for each aim. For the development of each algorithm, the data is divided into training and validation sets.

### Selecting a Model

Each model derived is dependent on the specific signal data as outputs from the specific instruments used in the system. The models were derived using training set data from representative subjects evaluated. In the model building process, in order for a variable (Z) to be a confounder of the biomarkers' relationship with the outcome (e.g., TBI+, TBI−), it must satisfy four conditions: (a) the predictor of interest (biomarkers, X) is not a cause of the confounding variable (Z); (b) the potential confounder (X) must be related to the outcome; (c) the potential confounder (Z) must be related to the predictor of interest (biomarkers, X); and (d) the coefficient of the predictor (βX) of interest must change when the confounder (Z) is included in the model. Potential confounding variables such as age, sex, time from injury until blood draw, and others are examined using these four steps to determine if they are truly confounders. Any confounding variables identified can be included as covariates in the models.

Furthermore, subgroups and interactions of the variables are examined within the intended use population. Pairwise interactions, or products of two predictor variables, are entered into the model provided the main effect terms of those predictor variables are also included the model. If the interaction is not significant, it is omitted from the model. Commonly, a backwards selection procedure is used for identifying which interactions should be retained in a model. First, a large model with all main effects and interaction terms is fit, and backwards selection is performed on all interaction terms, being careful to keep main effects of any significant interactions that are retained in the model. Then, backwards selection is performed on the remaining (main effects) predictors. Regarding subgroup analysis, if there are categorical variables or continuous variables that can be logically categorized into clinically meaningful groups that are identified as confounders, then stratified analyses may be conducted, provided that sufficient sample sizes for model building and validation are available.

The utilized model provides a probability/score that the subject has mild TBI. The area under the (receiver operating characteristic) curve (AUC) is reported using the fitted probabilities. A threshold is selected to ensure the model meets 85% sensitivity, where the specificity and confidence interval is reported. Accuracy is reported as the percentage of all subjects correctly classified as “mild TBI” versus “not mild TBI” using this threshold. As for measures of precision, confidence intervals are reported for all statistics. The probability of obtaining results at least as extreme as the observed results (p-value) is the area in the tail of the probability distribution. P-values comparing the AUC, specificity, and accuracy can also be reported. In the reported results, the likelihood of TBI for a given patient can be provided by reporting the fitted probability from frequentist-based models. Alternatively, Bayesian models can be fit instead of frequentist-based models, which would provide the likelihood along with a credible interval.

### Example Diagnosis and Prognosis Application

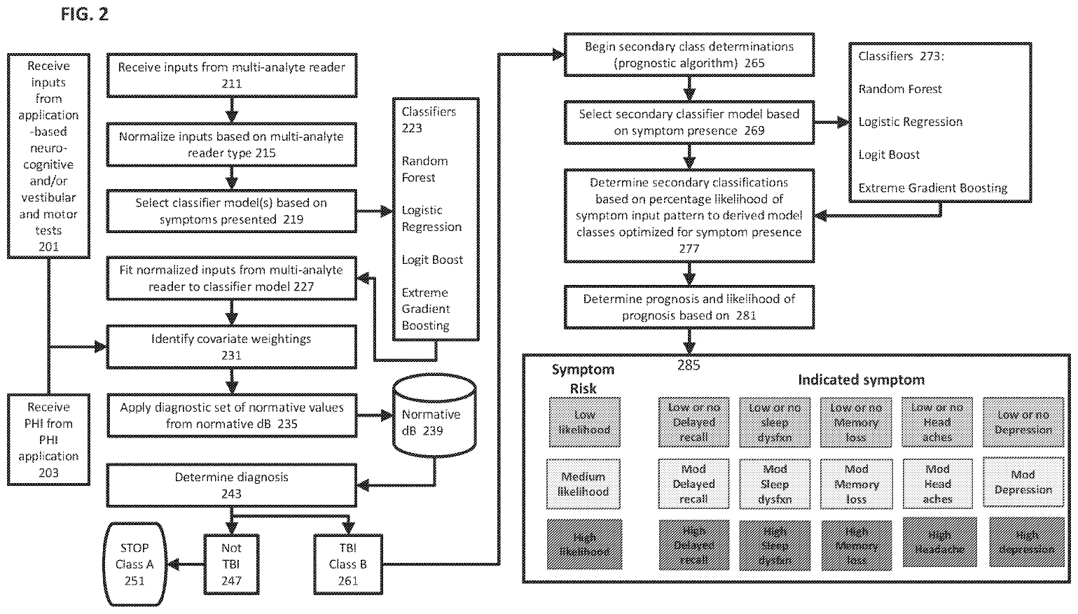

An example of the diagnosis and prognosis application 134 is shown graphically in FIG. 2, where the neurocognitive, vestibular, and/or motor test results from app 124 are received in block 201 by the BRAINBox server 130. Example neurocognitive, vestibular, and/or motor test results can include memory tests, processing speed tests, vestibular function tests, and noted symptom results. Eye movement and pupillometer test results can also be used. As outlined above, example neurocognitive test assessments are shown in FIG. 12. Performance testing is carried out using one or more of neurocognitive assessments 1205, 1210, 1215, 1220, 1225, 1230, 1235. An aggregated score from the neurocognitive test assessments is based on responses and performance on the neurocognitive assessments. The neurocognitive assessment results can be derived from the Flanker test, the Stroop Test, the Digit symbol Substitution Test, the Trailmaking Test, the Trails A and Trails B cognitive and executive function tests, and/or an immediate and delayed recall (short term memory) test. The patient's standardized score is determined as described above and is made available to the BRAINBox server 130.

In parallel, PHI information from PHI application 122 is also received in block 203 by the BRAINBox server 130. PHI application 122 can provide patient information, including Protected Health Information (PHI) that can include patient profile elements (e.g., sex, age, weight, etc.), demographic information, and other personal health information. In block 211, the BRAINBox server 130 also receives inputs from a multi-analyte reader, such as a POC reader 110, for example. The outputs from the multi-analyte reader can include biomarker signals which are processed signals from a detector device, derived, for example, from processed raw relative light units (fluorescence emission or luminescence, for example).

In block 215, the multi-analyte inputs to the diagnosis and prognosis application 134 are normalized based on the type of multi-analyte reader that was used. The normalized results are used as inputs to the classifier models, and a ranking of greatest accuracy, area under the curve (AUC), and relative risk is determined for each model. In block 219, a classifier model is selected from classifiers 223 based on the greatest accuracy, area under the curve, and relative risk as well as the symptoms presented by the patient. For example, the classifier models can treat different symptoms and cognitive performance differently, and a classifier model that provides the greatest accuracy for headaches and seizures may not provide the greatest accuracy for depression and anxiety or other symptoms or neurocognitive test results. Example classifiers are shown, but additional classifiers can also be used. Combinations of classifiers can also be used as well.

Exemplary embodiments of the invention select a classifier model based on symptoms presented by the patient and past examples of confirmed inputs (e.g., symptoms) and outputs (e.g., classification). The classification model uses a training data set with many examples of inputs (e.g., biomarker signals from multi-analyte readers 110, PHI information from PHI apps 122, neurocognitive/vestibular and motor test results from app 124) and outputs from which to learn. The models use the training dataset and calculate how to best map examples of input data to specific class labels, such as “subjects experiencing post-traumatic headache at 3 months after injury”, or “subjects having greater than a score of 9 on the patient health questionnaire-9, PHQ-9, for assessment of depressive symptoms, indicating moderate to severe depressive symptoms at the time of assessment”, or “having post-concussion syndrome with 3 or more symptoms at 3 months”. As such, the training dataset must be sufficiently representative of the patients and have many examples of each class label. The classification predictive models are selected based on prior, confirmed results.

In block 227, the normalized inputs from the multi-analyte reader are fit to the classifier(s). The inputs can be fit to the classifiers based on logistic regression, discriminant analysis, and other classification techniques. Logistic regression describes the data and explains the relationship between one dependent binary variable and one or more nominal, ordinal, interval, or ratio-level independent variables. Discriminant analysis classifies new observations into one of the known classification populations based on measured characteristics. Linear discriminant analysis computes “discriminant scores” for each observation to classify what response variable class the observation is in. These scores are obtained by finding linear combinations of the independent variables. Linear discriminant analysis assumes that the observations within each class are drawn from a multivariate Gaussian distribution and the covariance of the predictor variables are common across all levels of the response variable. Quadratic discriminant analysis provides an alternative approach that assumes that the observations from each class are drawn from a Gaussian distribution. However, quadratic discriminant analysis assumes that each class has its own covariance matrix. That is, the predictor variables are not assumed to have common variance across each of the levels of the response variable.

In block 231, covariate weightings are assigned to the multi-analyte results as well as to the results from the neurocognitive and/or vestibular and/or motor tests. Likewise, the PHI from the PHI application is also assigned covariate weightings in block 231.

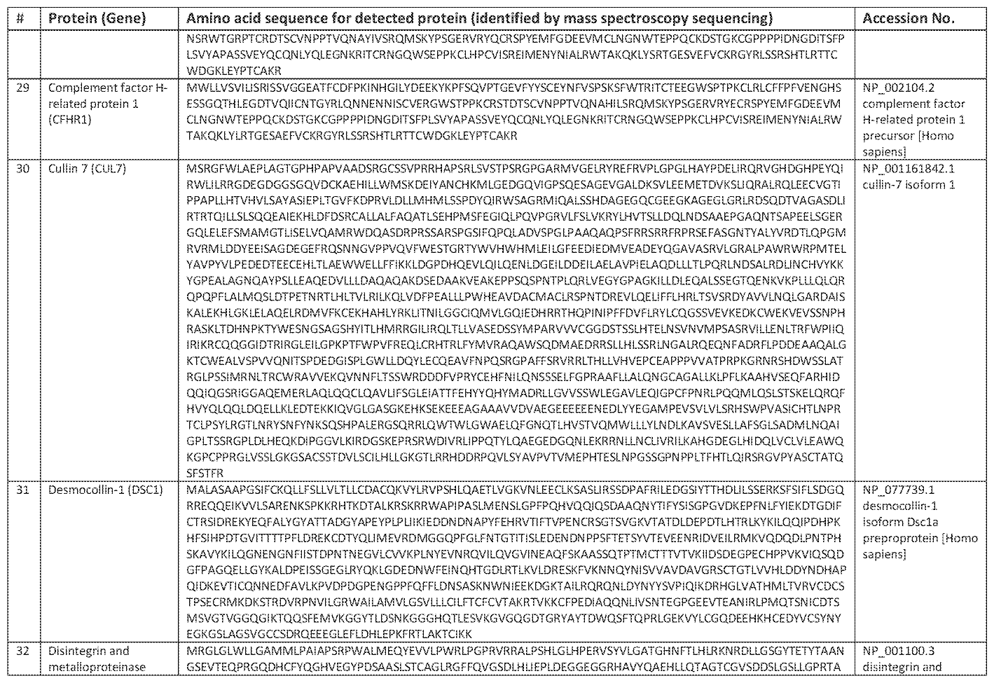

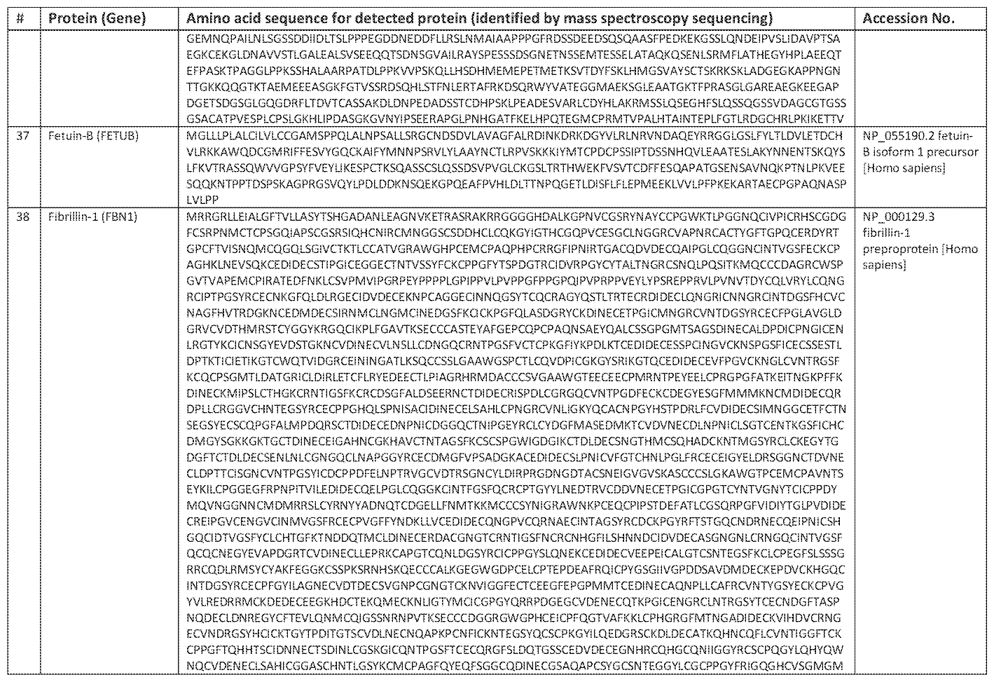

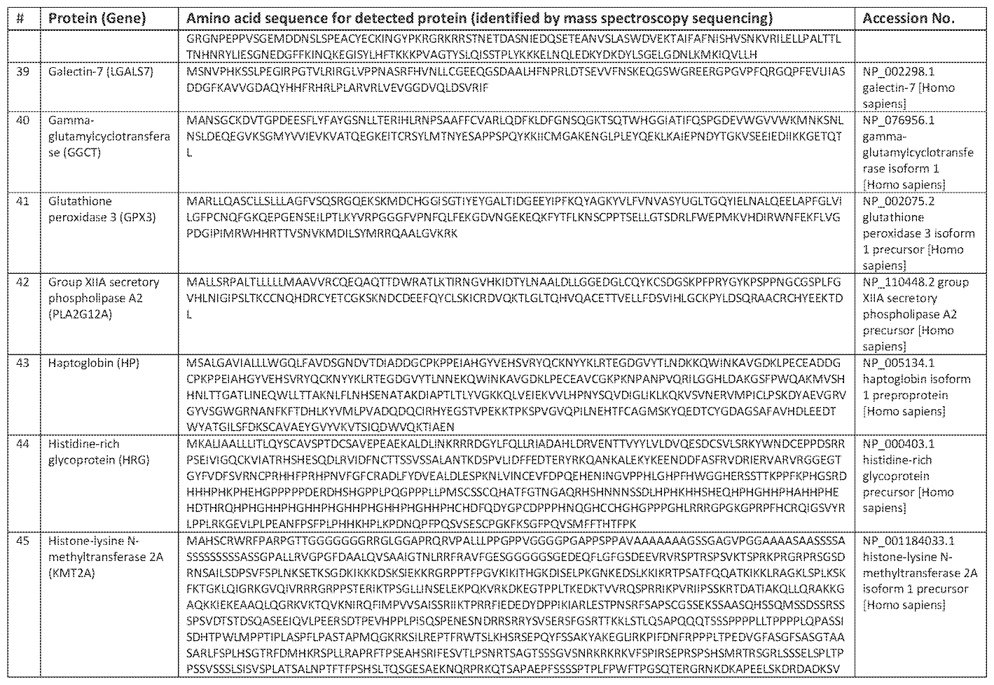

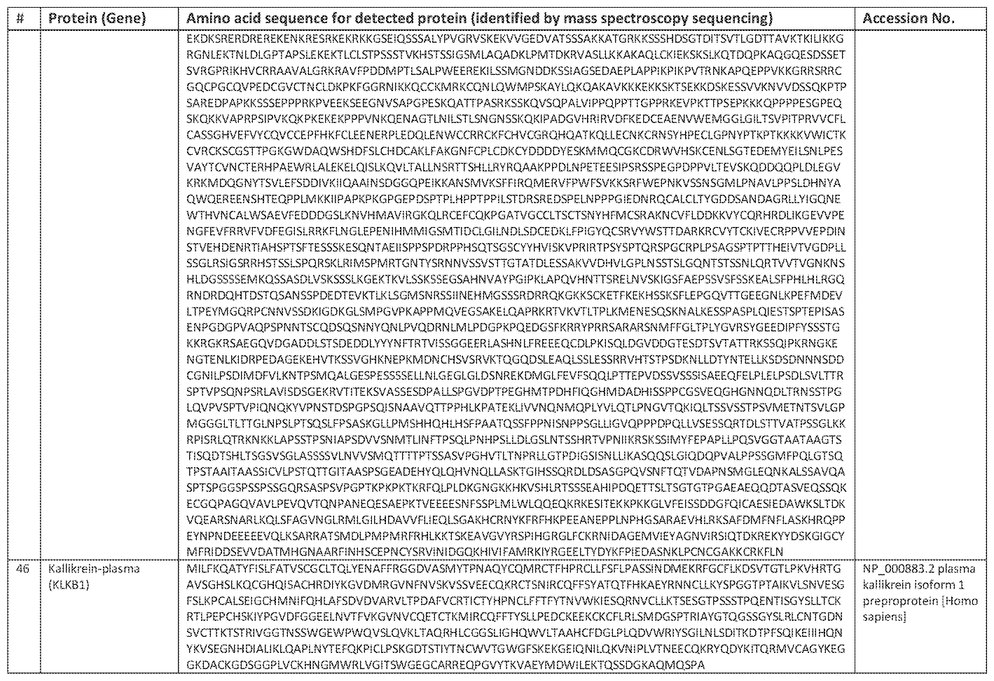

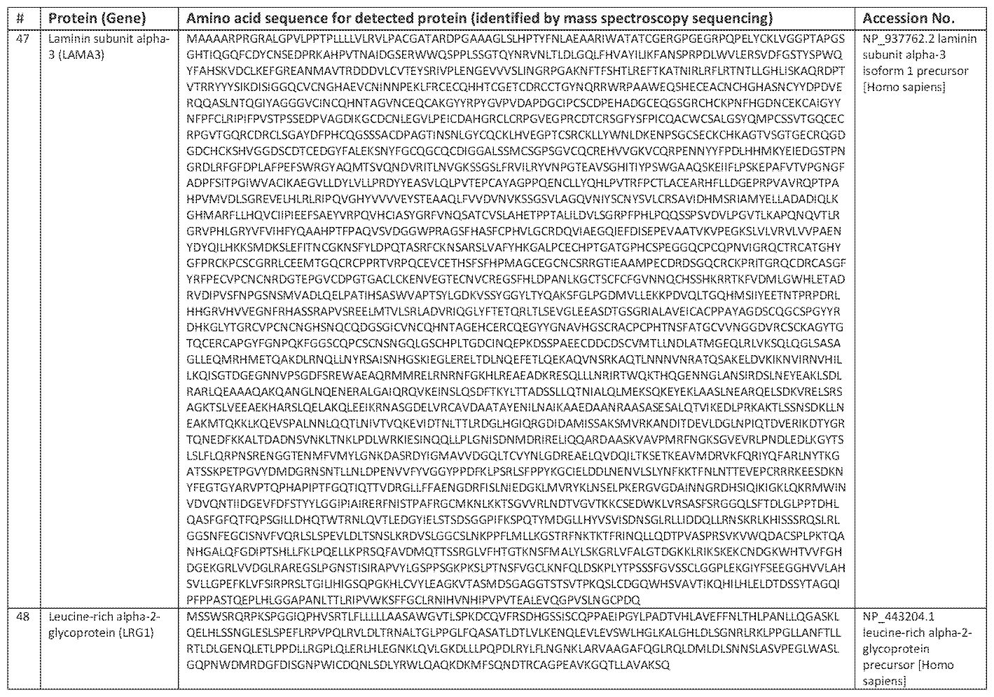

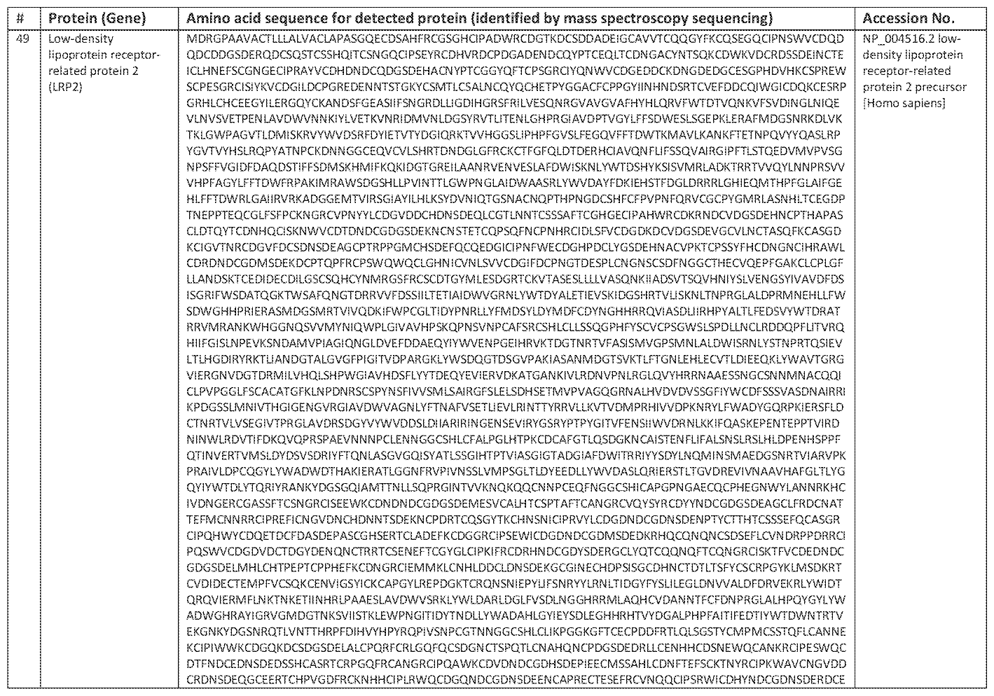

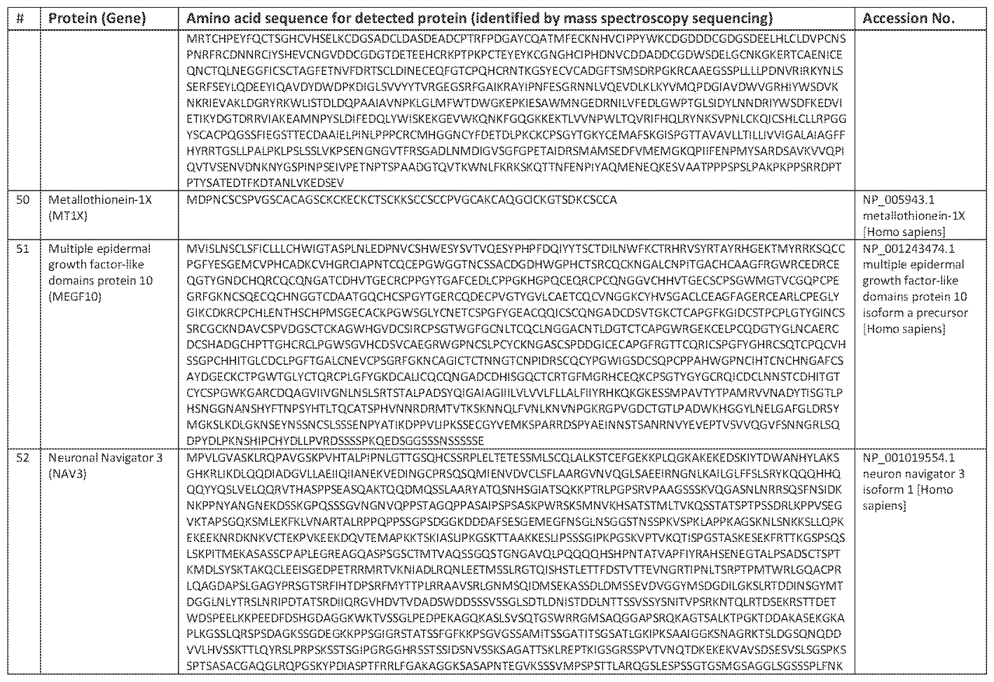

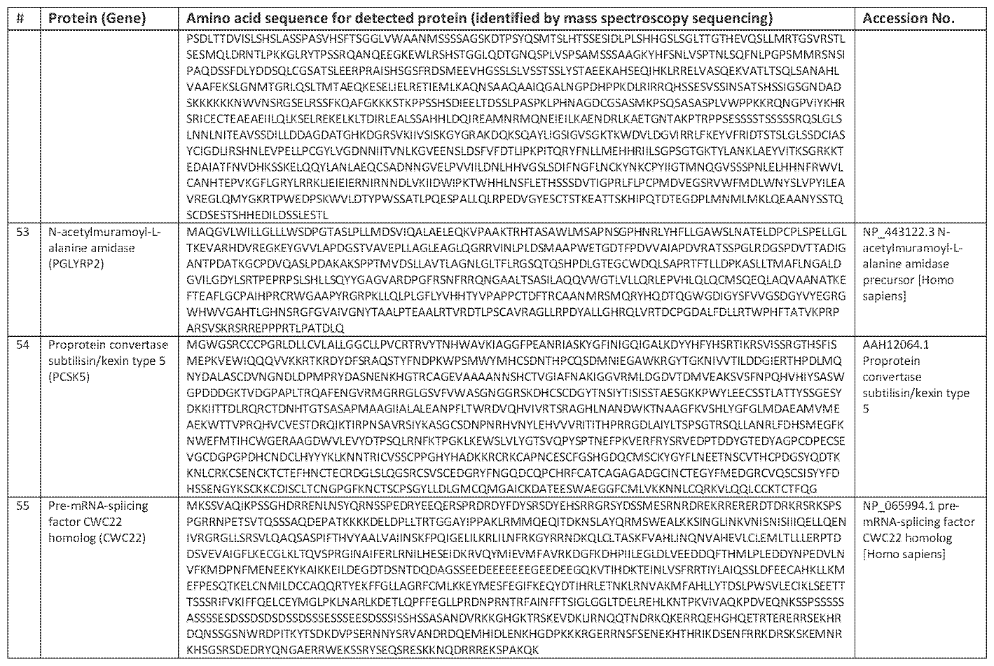

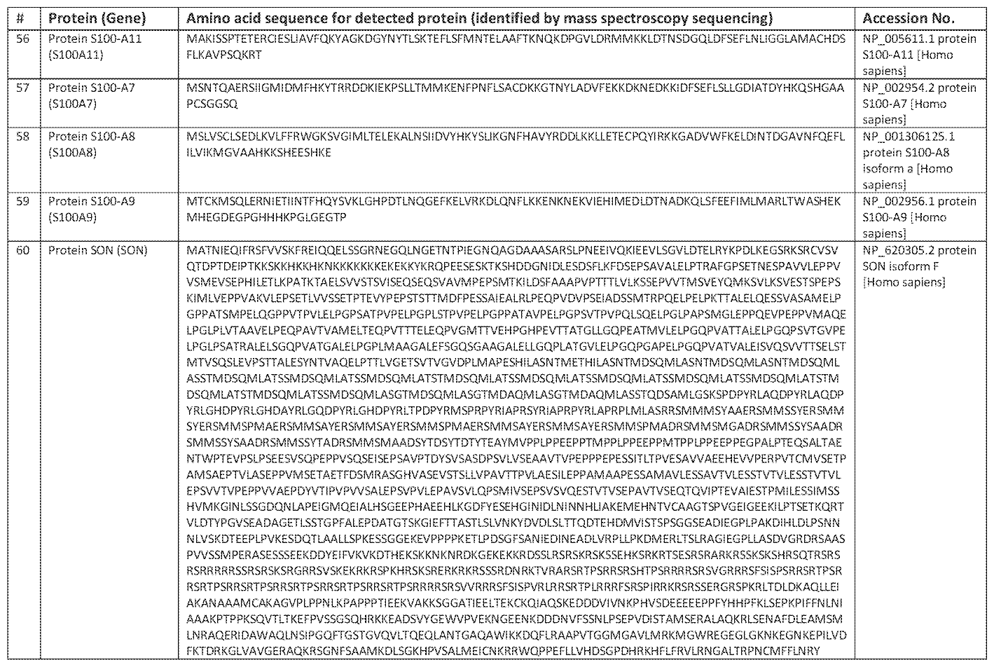

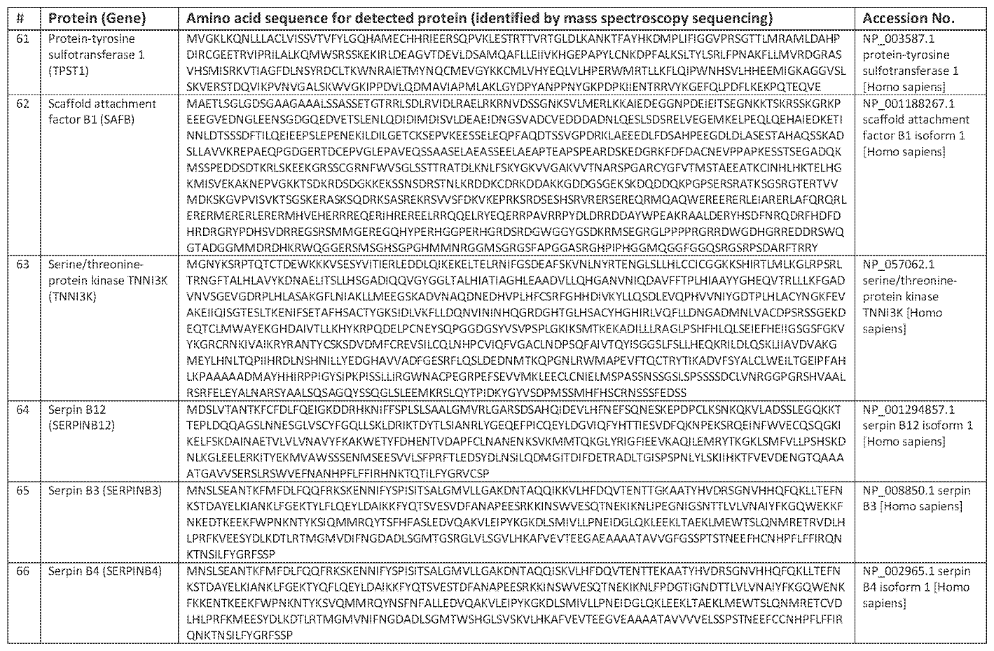

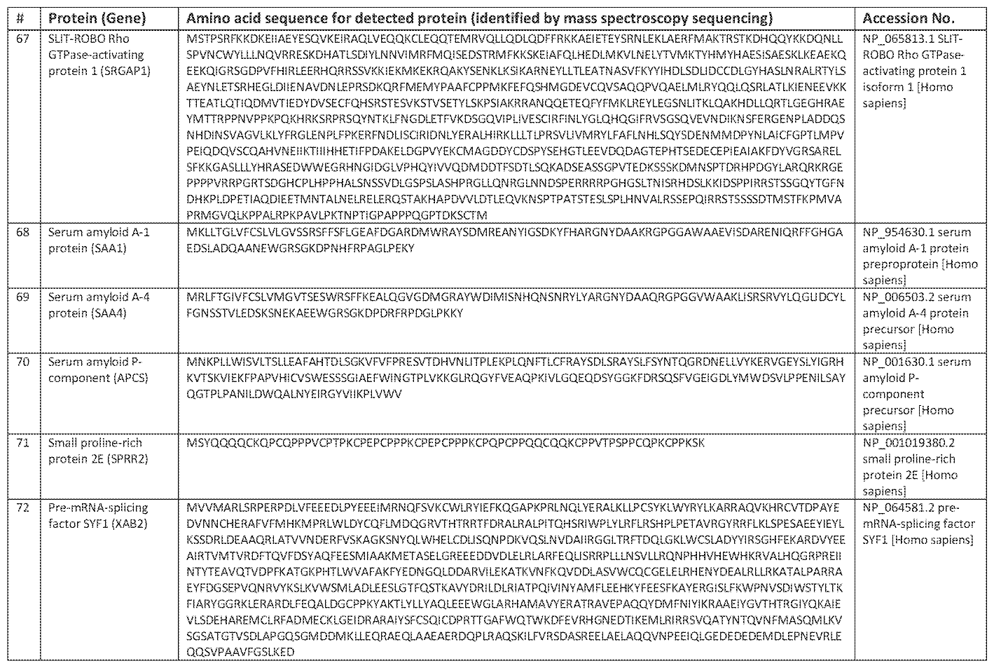

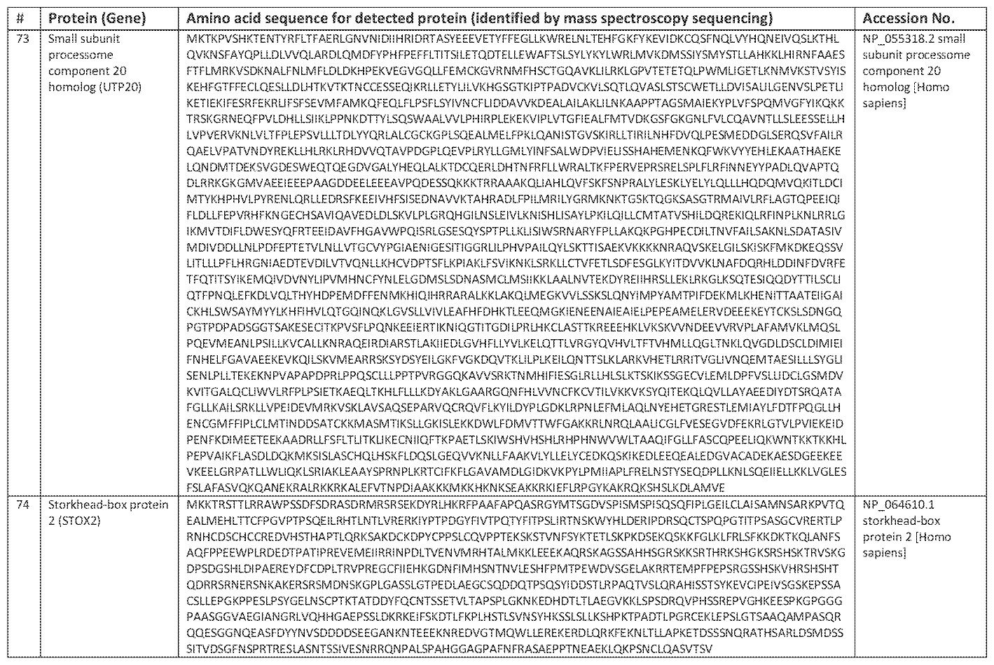

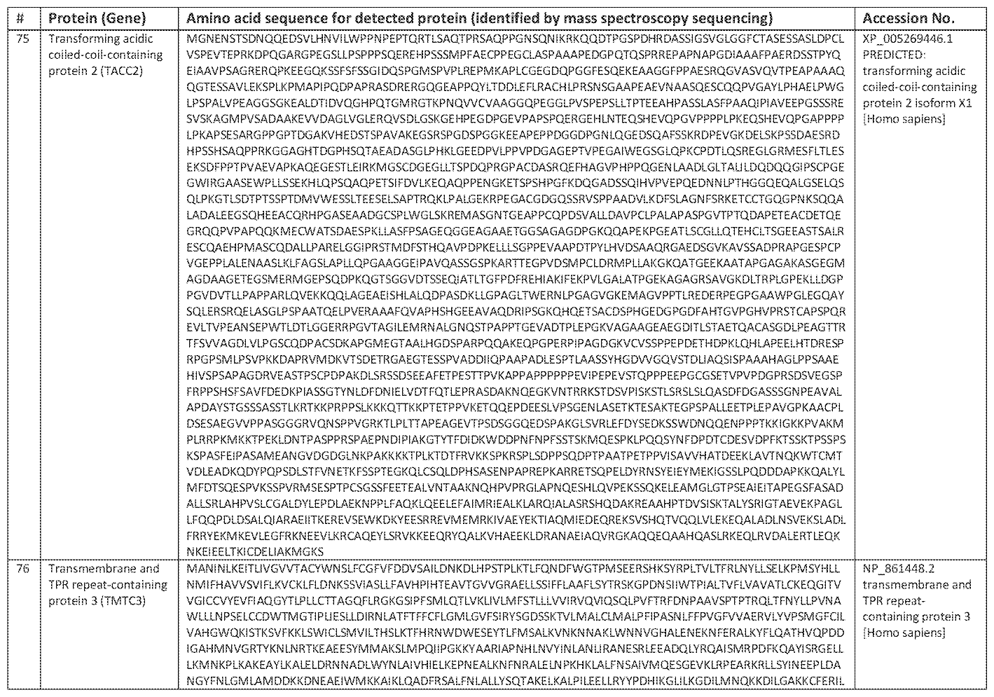

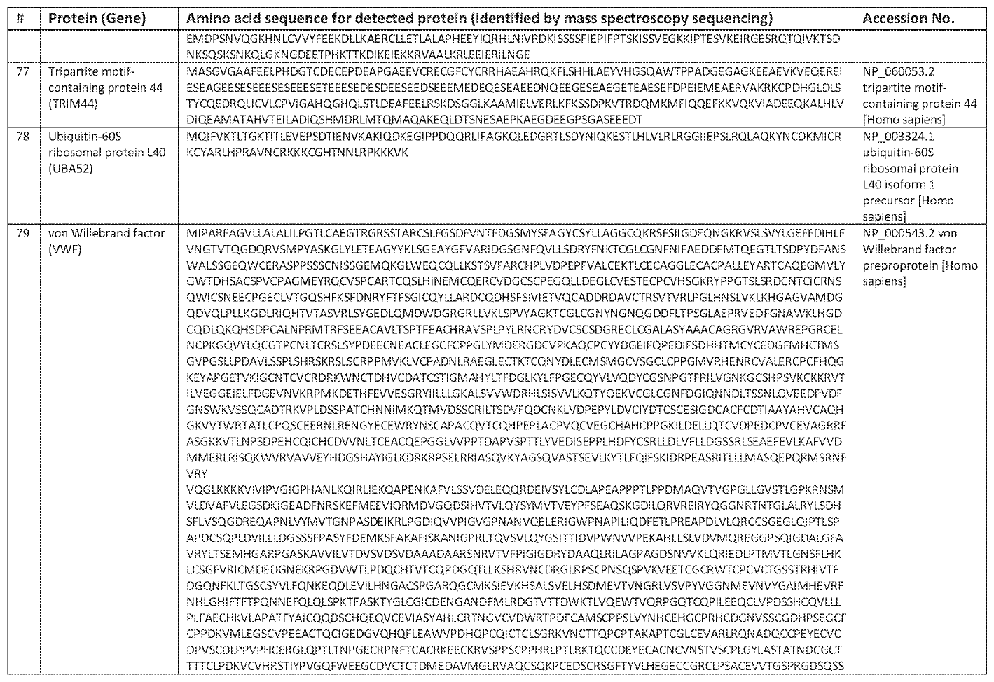

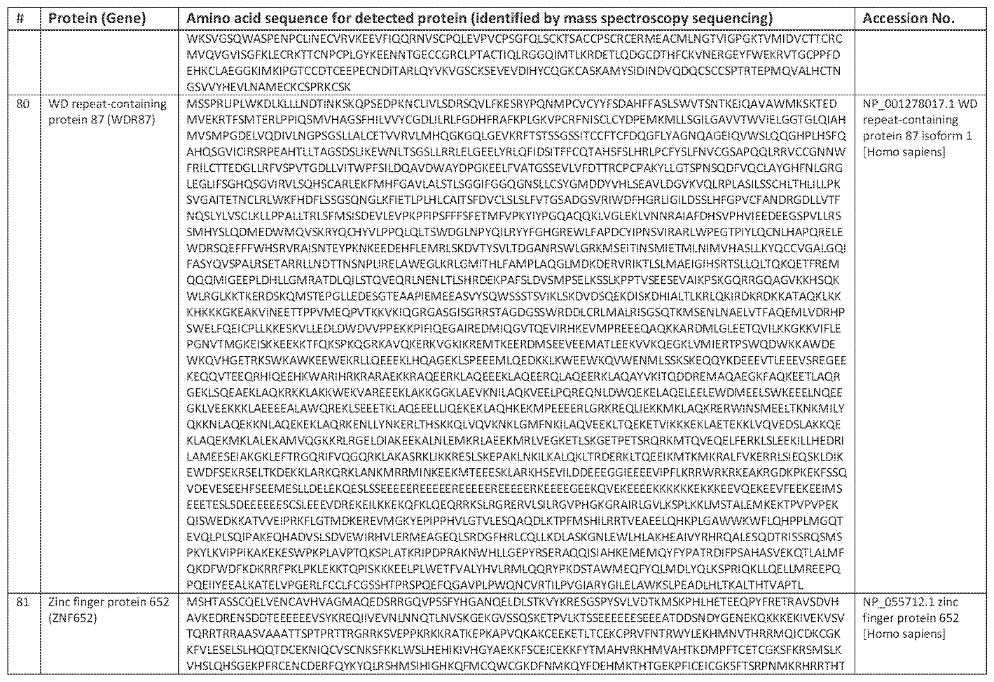

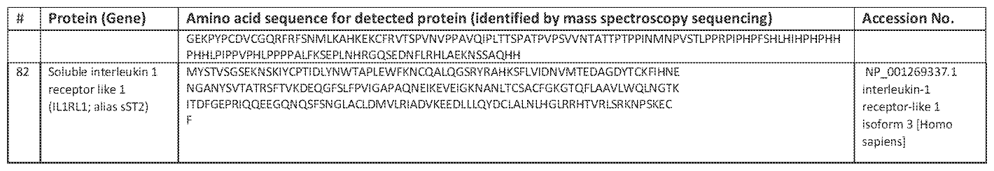

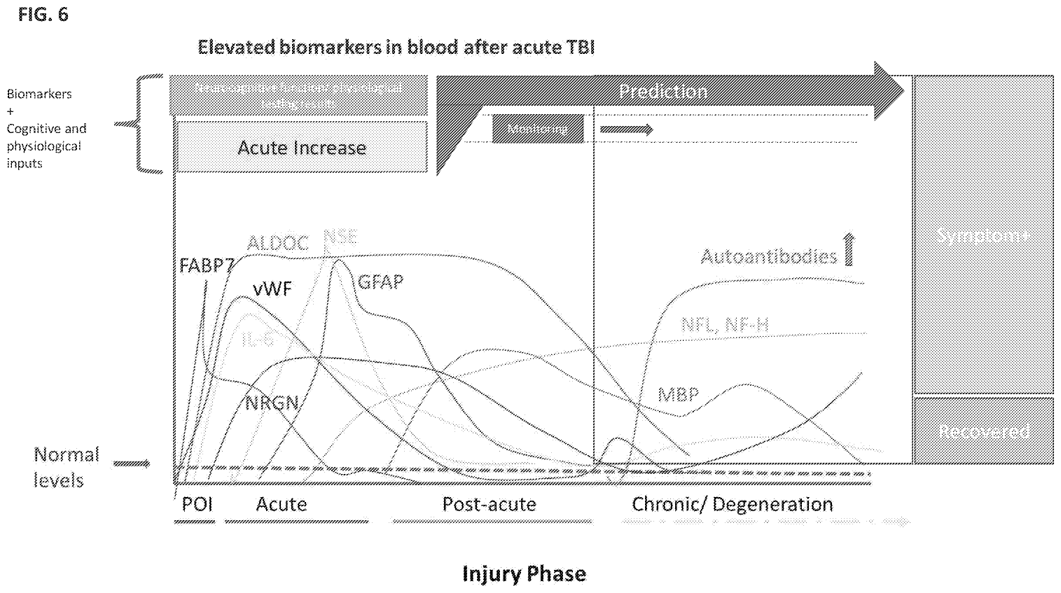

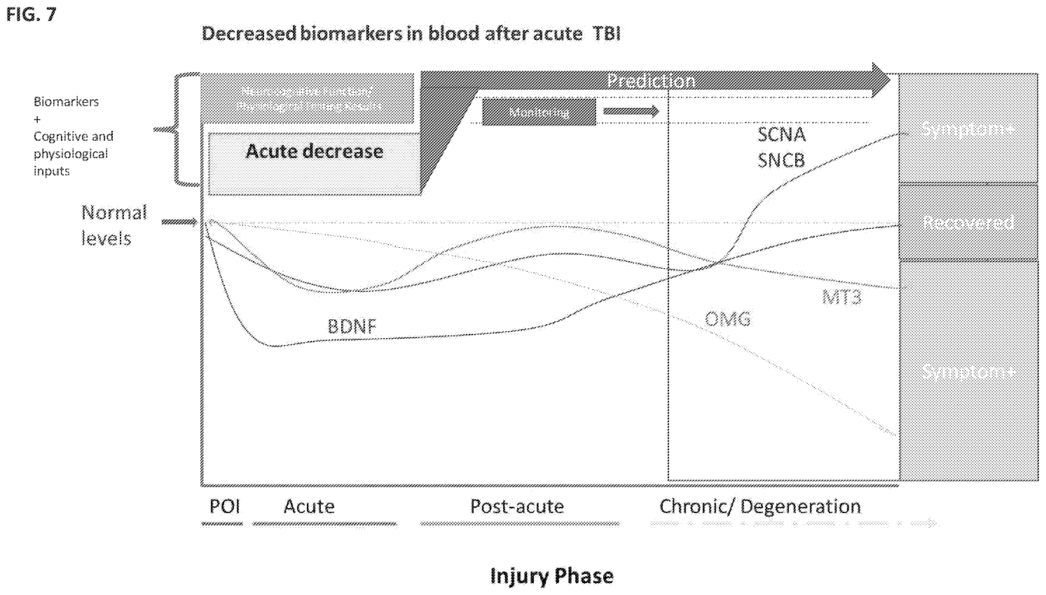

As shown further in FIG. 6, the covariate weightings can be based on elevated biomarkers (e.g., FABP7, ALDOC, vWF, IL6, NRGN, NSE, GFAP, and others) during the injury phase. Likewise, as shown in FIG. 7, the covariate weightings can be based on decreased biomarkers in the blood (e.g., BDNF, SCNA SCNB, MT3, OMG, and others) during the injury phase. Similarly, covariate weightings can be based on results from received PHI from the PHI application. The covariates for the biomarker tests are selected and ranked based on the degree to which they correlate or are linked to determined outcomes (e.g., diagnosis, prognosis) or to a particular clinical question.

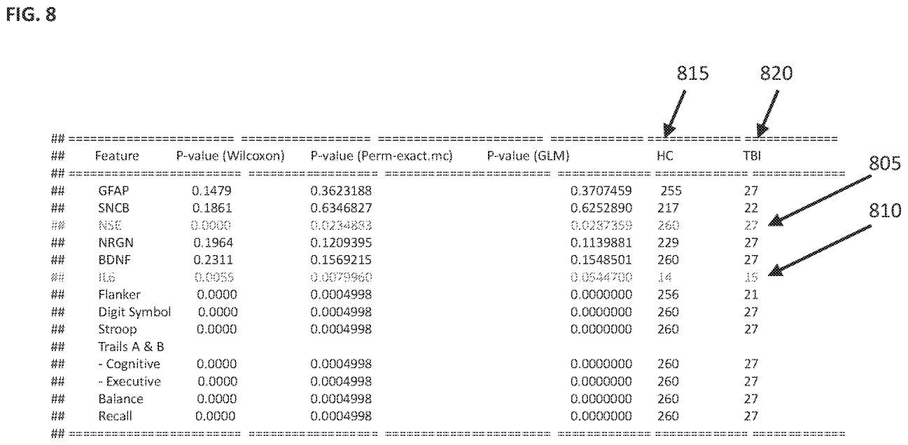

FIG. 8 shows differences in blood biomarker levels and standardized neurocognitive test scores between brain injured patients and uninjured control subjects using three different statistical methods. The biomarkers 805, 810 show statistically significant differences between the healthy control subjects (column 815) and those patients with traumatic brain injury (TBI) shown in column 820. The relative performance of different biomarkers can help determine covariates and covariate weighting.

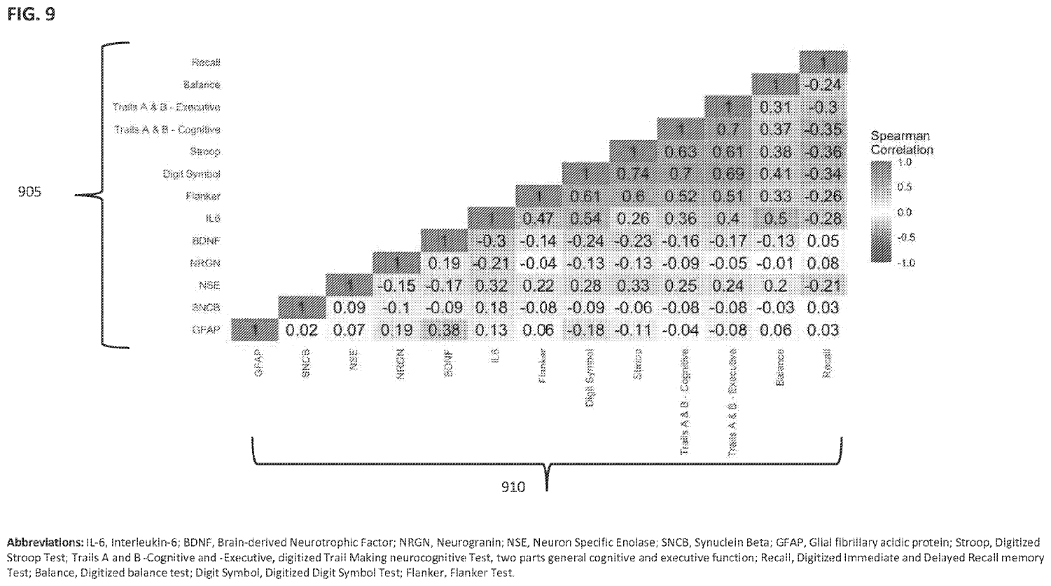

FIG. 9 provides additional (Spearman) correlation of blood biomarkers and standardized neurocognitive test scores in patients with mild TBI. The correlation shown in FIG. 9 provides an indication of the degree to which the biomarker and neurocognitive/vestibular assessment test results are tightly correlated.

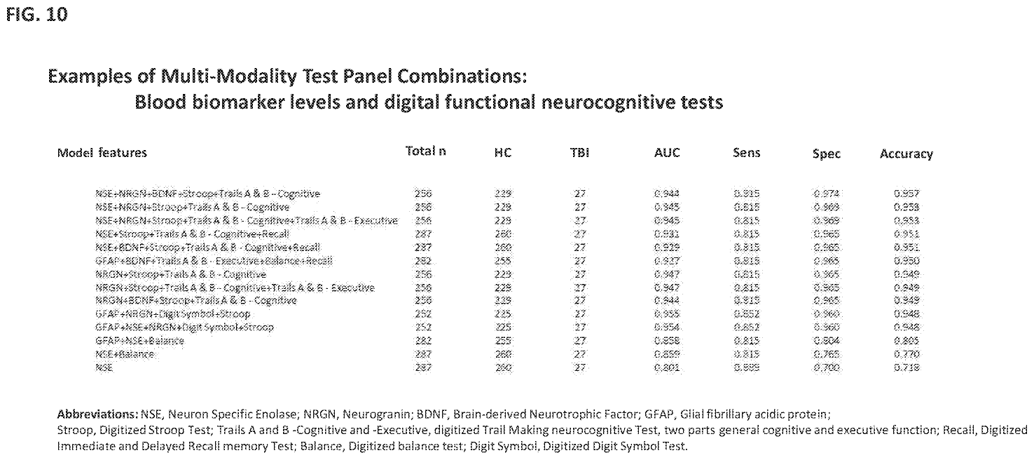

FIGS. 8 and 9 show differences in blood biomarker levels and standardized neurocognitive test scores between brain injured patients and uninjured control subjects based on individual biomarkers. For several different combinations, FIG. 10 reports the performance characteristics Sensitivity (Sens), Specificity (Spec), and Accuracy when combining different multi-modal features into one model predicting brain injured versus non-injured control subjects. As shown in FIG. 8, red highlighted elements 805, 810 for specific biomarkers show statistically significant biomarkers in this comparison, as examples (NSE, IL-6 having p values less than 0.05 for each statistic method used to compare data from the groups 815, 820, e.g., Wilcoxon rank sum test compares the median values of the groups). As individual markers are combined, the accuracy of the quantitative diagnostic test improves, as measured by the area under (a ROC) curve (AUC). For example, NRGN+Stroop+Trails A & B—Cognitive yields an AUC of 0.947, which results in an accuracy of 0.949. Similarly, GFAP+NSE+NRGN+Digit Symbol+Stroop has an AUC of 0.954 and an accuracy of 0.948. Blood biomarker levels and digital functional neurocognitive tests are shown along with the percentage of true positives (sensitivity—Sens) and the percentage of true negatives (specificity—Spec). As shown by the results in FIG. 10, the combination of biomarkers and neurocognitive tests provides additional confidence and capabilities in weighting covariates. FIG. 10 provides insight into the biomarkers that will be the most accurate predictors of TBI, and TBI severity.

Returning to FIG. 2, the identified co-variate weightings are then applied in block 235 against a diagnostic set of normative values from normative database 239, which includes reference values developed from clinical studies with TBI subtypes, general healthy, non-TBI individuals, and peripheral trauma control subjects.

Once the normative values are applied, the system 100 (shown in FIG. 1) determines a diagnosis for the patient in block 243 of FIG. 2. The diagnosis is based on a determined TBI score specified by the combination of the biomarker levels, the presented symptoms, the PHI, and the results of the neurocognitive/vestibular tests.

Furthermore, clinically significant subgroups are used to define and train each algorithm. For generating the BRAINBox TBI Score, all interactions are examined within the intended use population. For the algorithm that distinguishes mild TBI (true TBI+) from non-TBI (healthy or injured but not brain injured), the diagnostic scoring algorithm, the following derivation is used. Pairwise interactions, or products of two predictor variables, are entered into the model, provided the main effect terms of those predictor variables are also included the model. If the interaction is not significant, it will be omitted from the model. Commonly, a backwards selection procedure is used for identifying which interactions should be retained in a model. First, a large model with all main effects and interaction terms is fit and backwards selection is performed on all interaction terms, being careful to keep main effects of any significant interactions that are retained in the model. Backwards selection is performed on remaining (main effects) predictors. Regarding subgroup analysis, if there are categorical variables or continuous variables that can be logically categorized into clinically meaningful groups that are identified as confounders, then stratified analyses are conducted, provided that sufficient sample sizes for model building and cross-validation. The model provides a probability/score that the subject has mild TBI. The area under the receiver operating characteristic curve (AUC) is the resulting performance metric using the fitted probabilities. An AUC value preferably greater than 0.750 is a required performance level for all selected models, which is also defined by having at least 85% sensitivity and at least 75% specificity, and with greater than 75% accuracy. Accuracy is the percentage of all subjects correctly classified as “mild TBI” versus “non TBI” using this threshold. The TBI Score, our diagnostic measure, is the likelihood that a given patient has mild TBI, and is provided by reporting the fitted probability from frequentist-based models. Alternatively, Bayesian models could be fit instead of frequentist-based models, which would provide the likelihood along with a credible interval. Having established this model (TBI Score, the following is how raw data will become the TBI Score via the BRAINBox algorithm. Data from an independent patient is collected via a number of data inputs, which can be from more than one device. Biomarker measurements give a defined number of values, obtained from running a biofluid sample in a detection device, with onboard software for image capture and quantification of each biomarker signal. These biomarkers could be measured on more than one device. Raw data is standardized within the onboard device software (assay reader for measurement and normalization software), generating the individual preprocessed biomarker values. These data are transmitted to the processing data engine, which is cloud based. Data from the same subject is generated by having the subject perform a number of physiological or neurocognitive tests, which each generate standardized metrics that are the preprocessed data for those tests. Data from each test and device used is also communicated to the cloud-based data engine. Patient Health Information, including at a minimum age and sex, but also acute symptoms, are transmitted to the cloud by a device without preprocessing. The “TBI Score algorithm” draws together each signal input tied to a time stamp and a patient ID primary key, and integrates all preprocessed signals as primary model elements. The influence of each element in the model is adjusted by the specific predeveloped optimal covariate weighting, accomplished during model training. There are specific advantages of the selected elements, the combination of input types, is an iterative development process (which can be seen in the enhanced performance of the models, see the table shown in FIG. 10).

Returning to FIG. 2, the comparison of the model algorithm output (e.g., TBI Score) to the predefined normative threshold then provides a TBI score that is used in block 243 to determine a diagnosis. If the diagnosis is that the patient did not have a TBI in block 247, the process stops at block 251 and no additional analysis is performed.

If, however, the patient is diagnosed as having a TBI based on their TBI score in block 243, the process continues in block 261 as the severity and prognosis for that patient is determined. To determine a prognostic score, the secondary classification determinations are made in block 265. In block 269, a secondary classification model is selected from classifiers 273. As above, example classifiers are shown, but additional classifiers can also be used. Combinations of classifiers can also be used as well. In block 277, the normalized inputs from the multi-analyte reader are fit to the classifier(s) based on a likelihood of a symptom input pattern to be from the derived model classes, optimized covariates and their weightings, established as the signature for symptom presence. In block 281, a prognosis is determined and a likelihood (similar to a confidence score) of that prognosis is also determined. In block 285, the indicated symptoms are identified as well as their risk of manifestation.

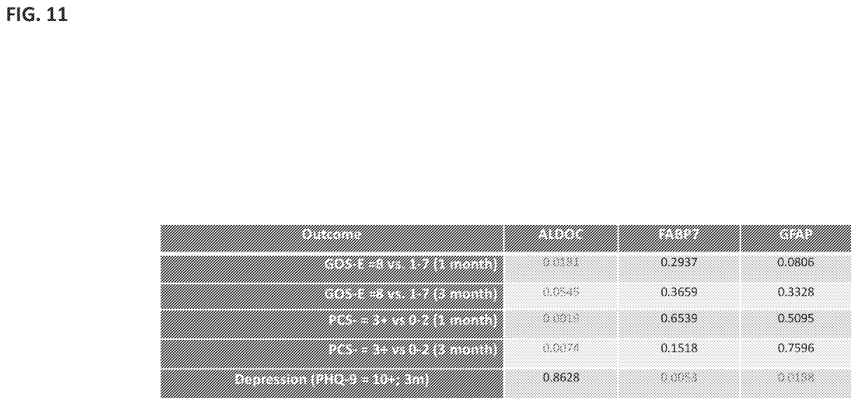

FIG. 11 shows an example correlation data in patient outcomes for 1, and 3 months, although 6 months or longer periods can also be determined in the same manner with data defining TBI subclasses at that timepoint post-injury. Biomarker levels from MSD assays were analyzed with respect to thresholds indicating one-month and three-month outcomes. FIG. 11 shows a summary of the results for three assay prototypes using appropriate cut-offs for mild TBI populations. FIG. 11 used a generalized linear regression modeling (GLM, p values shown). The three biomarker assays show discrimination for GOS-E and Rivermead PCS outcome groups.

In general, prognostic scores will be derived from algorithms trained using subgroups of patients defined by specific score thresholds in a time dependent manner. That is, for a combination of data inputs (e.g., A, B, C and D, along with functional neurocognitive test score A, B, C, and oculomotor score A and B), a separate model will be derived for each symptom category and derived for each post-injury time point (i.e., 14 days, 30 days or 90 days post-injury, as examples). The data will be evaluated to determine the prognostic output report. Results will be grouped into Low Risk and High Risk. If applicable, symptom categories may be combined into one overarching composite result, and defined as either Low Risk or High Risk. Please refer to the table shown in FIG. 10: Symptom Categories with Corresponding Neurocognitive (NC) and Neuropsychological (NP) Tests. NC/NP Test Symptom Category Composite Category/Result HIT-6 Headache Dizziness Handicap Inventory, Convergence Insufficiency Symptom Survey, BESS Motor Impairment (Balance, Dizziness/Vertigo, Visual Dysfunction) PROMIS Sleep Disturbance Short Form Sleep Disturbance GOS-E, BrainCheck Cognitive (Memory, Attention, Concentration, Executive Function) GAD-7, PCL-5, PHQ-9 and Perceived Stress Scale Psychological (Depression, Anxiety, Mood, Irritability, PTSD).

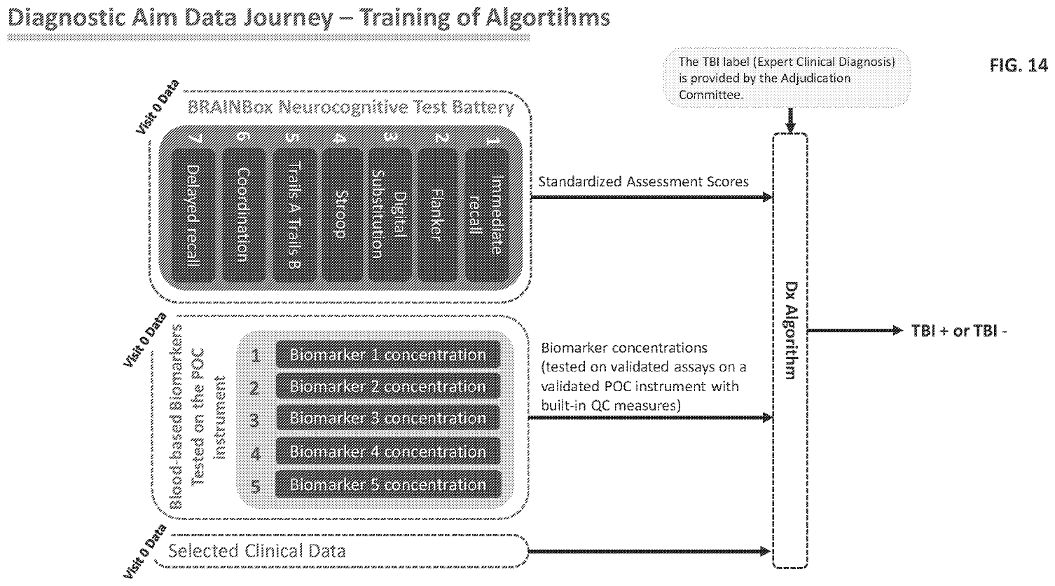

FIG. 14 shows an example of the process data elements, including individual biomarker values, acquired on a detection device, and tests or results from a separate instrument or device, are combined to generate a pattern of inputs used for determining whether a test subject or patient has TBI. A collection of such data elements from a population or cohort of TBI patients is used to train a diagnostic algorithm combining functional and biological (biomarkers, or BIBs) This gives a more comprehensive view of the patient's injury by combining biological factors and functional assessments into one diagnostic algorithm.

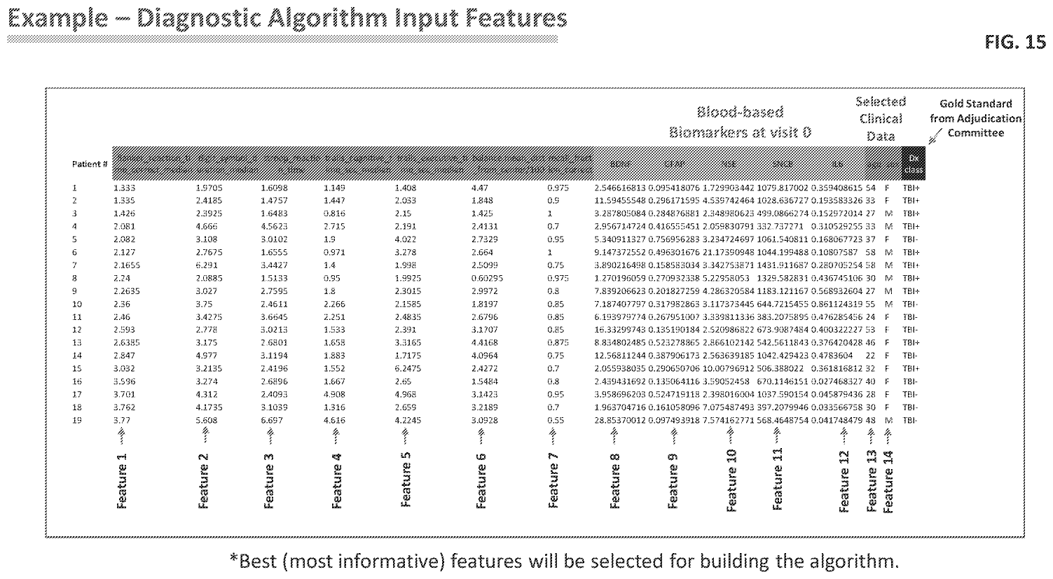

FIG. 15 shows an example. of the process data elements, with actual raw data, in the format received from the respective device, including individual biomarker values, acquired on a detection device, and tests or results from a separate instrument or device, are combined to generate a pattern of inputs used for determining whether a test subject or patient has TBI. A collection of such data elements from a population or cohort of TBI patients is used to train a diagnostic algorithm combining functional and biological (biomarkers, or BIBs) This gives a more comprehensive view of the patient's injury by combining biological factors and functional assessments into one diagnostic algorithm.

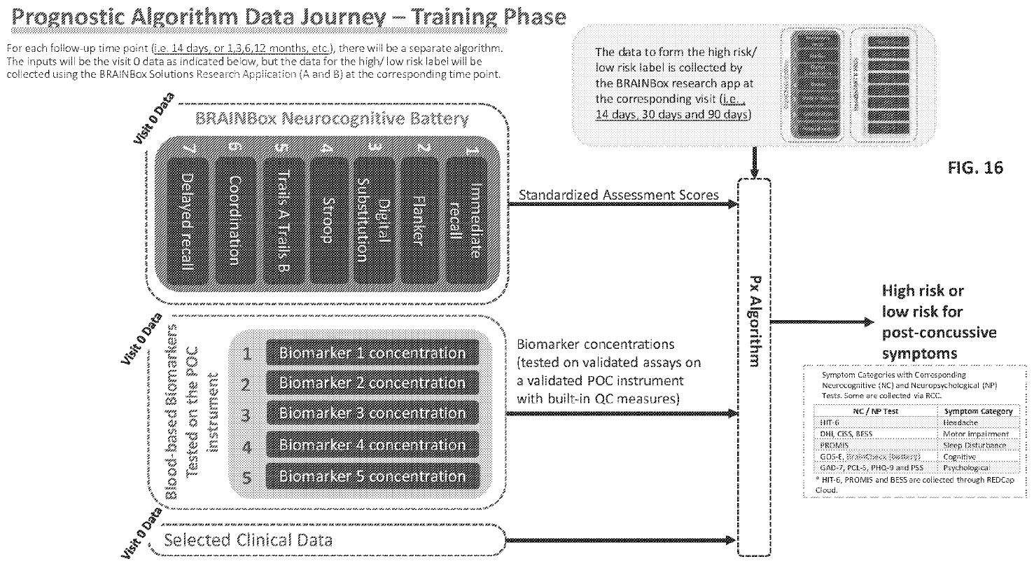

FIG. 16 shows an example of the process data elements, including individual biomarker values, acquired on a detection device, and tests or results from a separate instrument or device, are combined to generate a pattern of inputs used for determining whether a test subject known to have TBI or suspected to have TBI, has an increased likelihood of having a post-injury symptom defined by a predeveloped patient reported outcome assessment tool (e.g., PHQ9, a validated assessment instrument for depressive symptoms). A collection of such data elements from a population or cohort of TBI patients is used to train one or more prognostic algorithms by combining functional and biological (biomarkers, or BIBs), as well as other patient characteristics (sex, age, etc.). Such prognostic algorithms are specific to a defined outcome subgroup used to define the pattern int the input data elements that are associated with a patient subtype or subgroup. These outcomes could be defined by symptoms at a point after injury, or can encompass a time period or window of symptom assessment after injury. This gives a more comprehensive view of the patient's injury by combining biological factors and functional assessments into one prognostic algorithm.

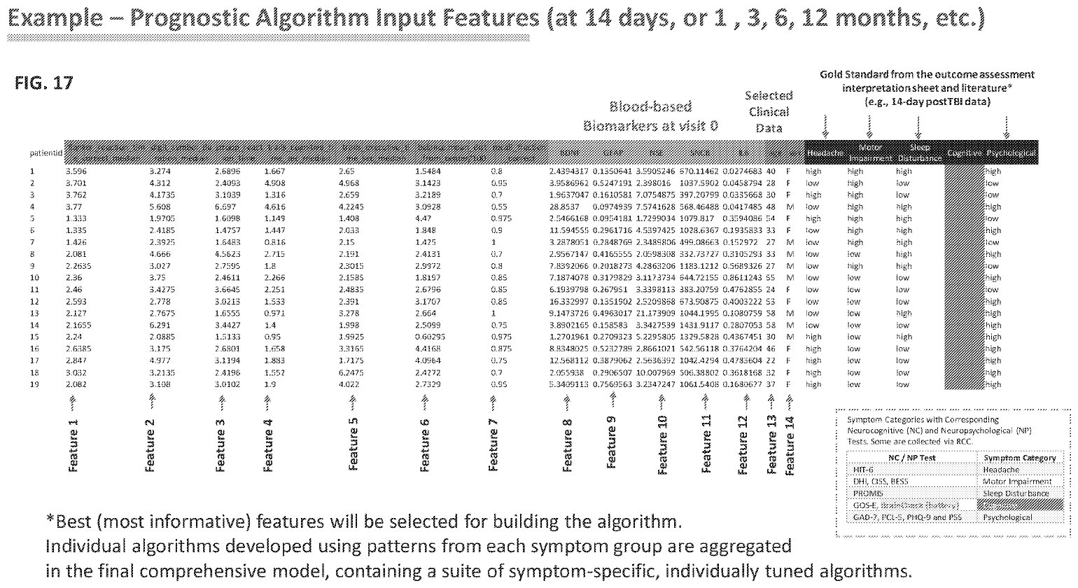

FIG. 17 shows an example of the process data elements showing actual raw data inputs as received from each device, in this example biomarker detection measurements from an assay reader and neurocognitive and balance assessment scores from a testing application on a tablet-based software. For this example, these include individual biomarker values, acquired on a detection device, and tests or results from a separate instrument or device, are combined to generate a pattern of inputs used for determining whether a test subject known to have TBI or suspected to have TBI, has an increased likelihood of having a post-injury symptom defined by a predeveloped patient reported outcome assessment tool). A collection of such data elements from a population or cohort of TBI patients is used to train one or more prognostic algorithms by combining functional and biological (biomarkers, or BIBs), as well as other patient characteristics (sex, age, etc.). Such prognostic algorithms are specific to a defined outcome subgroup used to define the pattern int the input data elements that are associated with a patient subtype or subgroup. These outcomes could be defined by symptoms at a point after injury, or can encompass a time period or window of symptom assessment after injury. This gives a more comprehensive view of the patient's injury by combining biological factors and functional assessments into one prognostic algorithm.

Several statistical modeling methods could be used to predict symptom category at each time point. Mixed effects models seem the most appropriate to account for correlated data from the same subject over time. If mixed effects models do not seem appropriate because the patient trajectories are not smooth, then at each individual time point models will be fit using methods such as multivariable logistic regression, random forests, support vector machines, gradient boosting, and Bayesian methods. According to Wolpert's no free lunch theorem, no method performs optimally for every modeling scenario. Therefore, different methods will be used to identify a best performing model. For each, the subjects will be divided into training and validation sets and the model will be derived using the training data and the validation data will be an independent set against which the model will be tested for generalizability.

In the model building procedure, in order for a variable (Z) to be a confounder of the biomarkers' relationship with the outcome (e.g., TBI+ high risk for symptoms, TBI+ low risk for symptoms), it must satisfy four conditions: (a.) the predictor of interest (biomarkers, X) is not a cause of the confounding variable (Z); (b.) the potential confounder (Z) must be related to the outcome; (c.) the potential confounder (Z) must be related to the predictor of interest (biomarkers, X); and (d.) the coefficient of the predictor (MX) of interest must change when the confounder (Z) is included in the model. Potential confounding variables such as age, sex, time from injury until blood draw, will be examined using these four steps to determine if they are truly confounders. Any confounding variables identified will be included in all models.

Subgroups and interactions will be examined within the intended use population. Pairwise interactions, or products of two predictor variables, can be entered into the model provided the main effect terms of those predictor variables are also included the model. If the interaction is not significant, it will be omitted from the model. A backwards selection procedure is used for identifying what interactions should be retained in a model. First, a large model with all main effects and interaction terms is fit and backwards selection is performed on all interaction terms, being careful to retain the main effects of any significant interactions. Then, backwards selection is performed on remaining (main effects) predictors. Regarding subgroup analysis, if there are categorical variables or continuous variables that can be logically categorized into clinically meaningful groups that are identified as confounders, stratified analyses may be conducted provided a sufficient sample size for model building and validation are available. To address potential sources of bias, variables that may affect symptoms post-injury could be examined such as treatment. Patient demographics will be included in the models or examined to determine if they are confounders. The models will provide the likelihood that the subject will experience the symptom at 14 days, 30 days and 90 days. A threshold will be selected to ensure the model meets 85% sensitivity, where the specificity, Positive Predictive Value and Negative Predictive Value will be reported. Accuracy will be reported as the percentage of all cases correctly classified at each time point.

### Temporal Capabilities

Exemplary embodiments of the system have the capability of testing in a static single test mode score, or in a “Delta” mode, in which a change in values over a defined interval is a component used to derive the score in the diagnosis and prognosis application. The delta mode is predetermined in the device settings and is an optional mode whereby, instead of a static reading of the physiological status of the patient, a change in values over time is used to determine the diagnosis or severity of a patient's injury.

### Remote Testing Component

Remote testing capability for the multianalyte reader and other components is envisioned such that subject in a remote testing location, including a home-based environment, can perform a simplified or automated test, have the results directly uploaded to the internet cloud-based processing software, and algorithms applied, and the result ported back to the user interface and/or physician, health care worker or other caregiver that is reviewing the result remotely. This is intended in the design of the system and supports telehealth and digital health settings and arrangements that are increasingly used for remote testing and healthcare delivery.

The BRAINBox TBI test multimodality system 100 provides a comprehensive solution to the diagnosis, prognosis and monitoring of TBI and concussion patients. By combining discreet neurocognitive and motor functional components, assessed in a real-time testing environment, with simultaneous assessment of biomarkers released into biofluids after brain cellular damage, the output score of the diagnosis and prognosis application offers clinicians a comprehensive panel of data to assist in the diagnosis of the full spectrum of brain injury.

### Detection of Brain Injury Biomarkers

### Detection by Immunoassay

In specific embodiments, the system includes a multi-analyte assay in which biomarkers are detected and/or measured by immunoassay. Immunoassay requires biospecific capture reagents/binding agent, such as antibodies, to capture the biomarkers. Many antibodies are available commercially. Antibodies also can be produced by methods well known in the art, e.g., by immunizing animals with the biomarkers (as antigens/immunogens). Biomarkers can be isolated from samples based on their binding characteristics. Alternatively, if the amino acid sequence of a polypeptide biomarker is known, the polypeptide can be synthesized and used to generate antibodies by methods well-known in the art.

Exemplary embodiments of the invention embrace traditional immunoassays including, for example, sandwich immunoassays, including ELISA or fluorescence-based immunoassays, immunoblots, Western Blots, as well as other enzyme immunoassays. Nephelometry is an assay performed in liquid phase, in which antibodies are in solution. Binding of the antigen to the antibody results in changes in absorbance. This change in absorbance is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments, the expression levels of the biomarkers employed herein are quantified by immunoassay, such as enzyme-linked immunoassay (ELISA) technology. In specific embodiments, the levels of expression of the biomarkers are determined by contacting the biological sample with antibodies, or antigen binding fragments thereof, that selectively bind to the biomarkers; and detecting binding of the antibodies, or antigen binding fragments thereof, to the biomarkers. In certain embodiments, the binding agents employed in the disclosed methods and compositions are labeled with a detectable moiety.

For example, the level of a biomarker in a sample can be assayed by contacting the biological sample with an antibody, or antigen binding fragment thereof, that selectively binds to the target biomarker (referred to as a capture molecule or antibody or a binding agent), and detecting the binding of the antibody, or antigen-binding fragment thereof, to the biomarker. The detection can be performed using a second antibody to bind to the capture antibody complexed with its target biomarker. A target biomarker can be an entire protein, or a variant or modified form thereof. Kits for the detection of biomarkers as described herein can include pre-coated strip plates, biotinylated secondary antibody, standards, controls, buffers, streptavidin-horse radish peroxidase (HRP), tetramethyl benzidine (TMB), stop reagents, and detailed instructions for carrying out the tests including performing standards.

Embodiments of the invention also provide methods for diagnosing brain injury in a subject, wherein the levels of expression of the biomarkers in a biological sample are determined simultaneously. For example, in one embodiment, methods are provided that comprise: (a) contacting a biological sample obtained from the subject with a plurality of binding agents that selectively bind to a plurality of biomarkers disclosed herein for a period of time sufficient to form binding agent-biomarker complexes; (b) detecting binding of the binding agents to the plurality of biomarkers, thereby determining the levels of expression of the biomarkers in the biological sample; and (c) comparing the levels of expression of the plurality of biomarkers in the biological sample with predetermined threshold values, wherein levels of expression of at least one of the plurality of polypeptide biomarkers above or below the predetermined threshold values indicates, for example, brain injury in the subject. Examples of binding agents that can be effectively employed in such methods include, but are not limited to, antibodies and antigen-binding fragments thereof, aptamers, lectins and the like.

In a further aspect, exemplary embodiments of the invention provide compositions that can be employed in the disclosed methods. In certain embodiments, such compositions comprise a solid substrate and a plurality of binding agents immobilized on the substrate, wherein each of the binding agents is immobilized at a different, indexable, location on the substrate and the binding agents selectively bind to a plurality of biomarkers disclosed herein. In a specific embodiment, the locations are pre-determined. In one embodiment, the binding agents selectively bind to a plurality of biomarkers described herein. Binding agents that can be employed in such compositions include, but are not limited to, antibodies, or antigen-binding fragments thereof, aptamers, lectins and the like.

In a related aspect, methods for assessing brain injury in a subject are provided, such methods comprising: (a) contacting a biological sample obtained from the subject with a composition disclosed herein for a period of time sufficient to form binding agent-polypeptide biomarker complexes; (b) detecting binding of the plurality of binding agents to the plurality of polypeptide biomarkers, thereby determining the levels of expression of the plurality of polypeptide biomarkers in the biological sample; and (c) comparing the levels of expression of the plurality of polypeptide biomarkers in the biological sample with predetermined threshold values, wherein levels of expression of at least one of the plurality of polypeptide biomarkers above or below the predetermined threshold values indicates brain injury status in the subject.

In yet another aspect, embodiments of the invention provide compositions comprising a solid substrate and a plurality of polypeptide biomarkers disclosed herein immobilized on the substrate, wherein each of the polypeptide biomarkers is immobilized at a different, indexable, location on the substrate. In certain embodiments, the plurality of polypeptide biomarkers includes Synuclein Beta (SNCB).

Although antibodies are useful because of their extensive characterization, any other suitable agent (e.g., a peptide, an aptamer, or a small organic molecule) that specifically binds a biomarker may be optionally used in place of the antibody in the above described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

In specific embodiments, the assay performed on the biological sample can comprise contacting the biological sample with one or more capture agents (e.g., antibodies, peptides, aptamer, etc., combinations thereof) to form a biomarker capture agent complex. The complexes can then be detected and/or quantified. A subject can then be identified as having brain injury based on a comparison of the detected/quantified/measured levels of biomarkers to one or more reference controls as described herein.

In one method, a first, or capture, binding agent, such as an antibody that specifically binds the biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker (or to the bound capture antibody) is then used to detect binding of the polypeptide biomarker to the capture antibody. The detection antibody is preferably conjugated, either directly or indirectly, to a detectable moiety. Examples of detectable moieties that can be employed in such methods include, but are not limited to, chemiluminescent and luminescent agents; fluorophores such as fluorescein, rhodamine and eosin; radioisotopes; colorimetric agents; and enzyme-substrate labels, such as biotin.

In another embodiment, the assay is a competitive binding assay, wherein labeled biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in U.S. Patent Application Publication No. US 2010/0093557 A1. Methods for the automation of immunoassays are well known in the art and include, for example, those described in U.S. Pat. Nos. 5,885,530, 4,981,785, 6,159,750 and 5,358,691.

The presence of several different polypeptide biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in U.S. Patent Application Publication Nos. US 2010/0093557A1 and US 2010/0190656A1, the entire disclosures of which are specifically incorporated by reference herein.

Multiplex arrays in several different formats based on the utilization of, for example, flow cytometry, chemiluminescence or electro-chemiluminescence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

### Detection by Mass Spectrometry

In one aspect, the biomarkers may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap, hybrids or combinations of the foregoing, and the like.

In particular embodiments, the biomarkers are detected using selected reaction monitoring (SRM) mass spectrometry techniques. Selected reaction monitoring (SRM) is a non-scanning mass spectrometry technique, performed on triple quadrupole-like instruments and in which collision-induced dissociation is used as a means to increase selectivity. In SRM experiments two mass analyzers are used as static mass filters, to monitor a particular fragment ion of a selected precursor ion. The specific pair of mass-over-charge (m/z) values associated to the precursor and fragment ions selected is referred to as a “transition” and can be written as parent m/z→fragment m/z (e.g. 673.5→534.3). Unlike common MS based proteomics, no mass spectra are recorded in a SRM analysis. Instead, the detector acts as counting device for the ions matching the selected transition thereby returning an intensity distribution over time. Multiple SRM transitions can be measured within the same experiment on the chromatographic time scale by rapidly toggling between the different precursor/fragment pairs (sometimes called multiple reaction monitoring, MRM). Typically, the triple quadrupole instrument cycles through a series of transitions and records the signal of each transition as a function of the elution time. The method allows for additional selectivity by monitoring the chromatographic co-elution of multiple transitions for a given analyte.

The terms SRM/MRM are occasionally used also to describe experiments conducted in mass spectrometers other than triple quadrupoles (e.g. in trapping instruments) where upon fragmentation of a specific precursor ion a narrow mass range is scanned in MS2 mode, centered on a fragment ion specific to the precursor of interest or in general in experiments where fragmentation in the collision cell is used as a means to increase selectivity. In this application, the terms SRM and MRM or also SRM/MRM can be used interchangeably, since they both refer to the same mass spectrometer operating principle. As a matter of clarity, the term MRM is used throughout the text, but the term includes both SRM and MRM, as well as any analogous technique, such as e.g. highly-selective reaction monitoring, hSRM, LC-SRM or any other SRM/MRM-like or SRM/MRM-mimicking approaches performed on any type of mass spectrometer and/or, in which the peptides are fragmented using any other fragmentation method such as e.g. CAD (collision-activated dissociation (also known as CID or collision-induced dissociation), HCD (higher energy CID), ECD (electron capture dissociation), PD (photodissociation) or ETD (electron transfer dissociation).

In another specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein.

In an alternative embodiment, the mass spectrometric technique comprises surface enhanced laser desorption and ionization or “SELDI,” as described, for example, in U.S. Pat. Nos. 6,225,047 and 5,719,060, which are incorporated herein by reference. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker panel, pursuant to exemplary embodiments of the invention.

In another mass spectrometry method, the biomarkers can be first captured on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

### Detection by Electrochemiluminescent Assay

In several embodiments, the biomarker may be detected by means of an electrochemiluminescent assay developed by Meso Scale Discovery (Gaithersburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ^{˜}620 nm, eliminating problems with color quenching. See, e.g., U.S. Pat. Nos. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also, U.S. Patent Application Publication Nos. 2009/0170121; 2009/006339; 2009/0065357; 2006/0172340; 2006/0019319; 2005/0142033; 2005/0052646; 2004/0022677; 2003/0124572; 2003/0113713; 2003/0003460; 2002/0137234; 2002/0086335; and 2001/0021534; all of which are incorporated by reference in their entireties.

### Other Methods for Detecting Biomarkers

The biomarkers can be detected by any other known suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Invitrogen Corp. (Carlsbad, Calif.), Affymetrix, Inc. (Fremont, Calif.), Zyomyx (Hayward, Calif.), R&D Systems, Inc. (Minneapolis, Minn.), Biacore (Uppsala, Sweden) and Procognia (Berkshire, UK). Examples of such protein biochips are described in the following patents or published patent applications: U.S. Pat. Nos. 6,537,749; 6,329,209; 6,225,047; 5,242,828; International PCT Publication No. WO 00/56934; and International PCT Publication No. WO 03/048768; all of which are incorporated by reference in their entireties.

In a particular embodiment, Exemplary embodiments of the invention comprises a microarray chip. More specifically, the chip comprises a small wafer that carries a collection of binding agents bound to its surface in an orderly pattern, each binding agent occupying a specific position on the chip. The set of binding agents specifically bind to each of the one or more one or more of the biomarkers described herein. In particular embodiments, a few micro-liters of blood serum or plasma are dropped on the chip array. Biomarker proteins present in the tested specimen bind to the binding agents specifically recognized by them. Subtype and amount of bound mark is detected and quantified using, for example, a fluorescently-labeled secondary, subtype-specific antibody. In particular embodiments, an optical reader is used for bound biomarker detection and quantification. Thus, a system can comprise a chip array and an optical reader. In other embodiments, a chip is provided.

### Determination of a Subject's Brain Injury Status

Exemplary embodiments of the invention generally relate to the use of biomarkers to assess brain injury, such as traumatic brain injury (TBI) or concussion. More specifically, neurocognitive and/vestibular tests, PHI information, as well as the biomarkers can be used in diagnostic and determinative tests and methods to determine, qualify, and/or assess brain injury, for example, to assess brain injury, in an individual, subject, or patient. More specifically, the biomarkers to be detected in assessing brain injury status include, but are not limited to any of the biomarkers disclosed herein.

### Biomarker Panels

According to exemplary embodiments of the invention, the biomarkers can be used in panels of several biomarkers in diagnostic tests to assess, determine, and/or qualify (used interchangeably herein) brain injury in a patient. The phrase “brain injury status” includes any distinguishable manifestation of brain injury, as the case may be, including not having brain injury. For example, brain injury status includes, without limitation, brain injury or non-injury in a patient, the stage or severity of brain injury, the progress of brain injury (e.g., progress of brain injury over time), or the effectiveness or response to treatment of brain injury (e.g., clinical follow up and surveillance of brain injury after treatment). Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the biomarker panels of the invention may show a statistical difference in different brain injury statuses of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9.

The biomarkers can be differentially present in UI (NC or non-brain injury) and brain injury, and, therefore, are useful in aiding in the determination of brain injury status. In certain embodiments, the biomarkers are measured in a patient sample using the methods described herein and compared, for example, to predefined biomarker levels/ratios and correlated to brain injury status. In particular embodiments, the measurement(s) may then be compared with a relevant diagnostic amount(s), cut-off(s), or multivariate model scores that distinguish a positive brain injury status from a negative brain injury status. The diagnostic amount(s) represents a measured amount of a biomarker(s) above which or below which a patient is classified as having a particular brain injury status. For example, if the biomarker(s) is/are up-regulated compared to normal (e.g., a control), then a measured amount(s) which is above the diagnostic cutoff(s) provides an assessment of brain injury status. Alternatively, if the biomarker(s) is/are down-regulated, then a measured amount(s) at or below the diagnostic cutoff(s) provides an assessment of brain injury status. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of biomarkers in a statistically significant number of samples from patients with the different brain injury statuses, and drawing the cut-off to suit the desired levels of specificity and sensitivity.

In other embodiments, the relative or normalized amounts biomarkers to each other are useful in aiding in the determination of brain injury status. In certain embodiments, the biomarker ratios are indicative of diagnosis. In other embodiments, a biomarker ratio can be compared to another biomarker ratio in the same sample or to a set of biomarker ratios from a control or reference sample.

Furthermore, in certain embodiments, the values measured for markers of a biomarker panel are mathematically combined and the combined value is correlated to the underlying diagnostic question. Biomarker values may be combined by any appropriate state of the art mathematical method. Mathematical methods useful for correlating a marker combination to a brain injury status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods, including extreme gradient boosting (XG Boost), Generalized Linear Models (e.g., Logistic Regression, Linear Mixed Effects), Principal Components based Methods (e.g., SIMCA), Generalized Additive Models, Fuzzy Logic based Methods, Neural Networks, Genetic Algorithms based Methods, and variations and combinations thereof. In one embodiment, the method used in correlating a biomarker combination, e.g. to assess brain injury, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

### Determining Risk of Brain Injury

Exemplary embodiments of the invention provide methods for determining the risk of brain injury in a patient. Neurocognitive test results, vestibular test results, motor test results combined with biomarker percentages, ratios, levels, amounts, or patterns are characteristic of various risk states of a TBI symptom developing over a period of time from the injury. The risk states may be classified as high, medium, or low. The risk of developing symptoms indicative of a brain injury is determined by evaluating the neurocognitive test results, vestibular test results, and motor test results and measuring the relevant biomarkers for a particular symptom while accounting for PHI information. The combination of results is then submitted to a classification algorithm and/or are compared to a reference amount, such as a predefined level or pattern of biomarkers that is associated with the particular symptom and a particular risk level for developing that symptom.

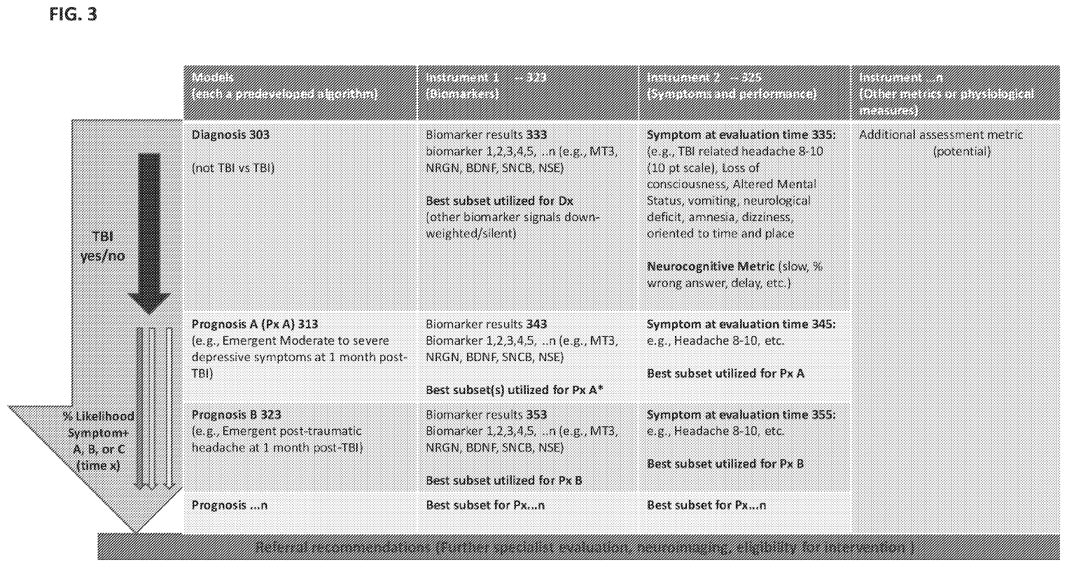

A number of examples are shown in FIG. 3, including models developed using covariates to find optimal weightings, measured in relationship to established control population ranges. In the first row, a binary diagnosis 303 is made between a TBI or not a TBI using outputs 333 from the assay reader 110 (a.k.a. “instrument 1”), outputs 335 from neurocognitive/vestibular testing app 124 (a.k.a. “instrument 2”), and outputs from PHI app 122 (a.k.a. “instrument n”). The outputs of these “instruments” are used as inputs to the diagnosis and prognosis application 134. Diagnosis 303 is made based upon the inputs 333, 335, and other inputs to the diagnosis and prognosis application 134.

Further, the biomarker results 343 from the multi-analyte assay shown in the column labeled “Instrument 1,” and the neurocognitive/vestibular/motor test results 345 shown in the column labeled “Instrument 2,” as well as PHI data or other test results, metrics, or physiological measures are used in the prognosis determination algorithms and processes of exemplary embodiments of the invention. Based on the biomarkers present 343 and the symptom(s) at evaluation time 345, a prognosis 313 in FIG. 3 can be made for particular symptoms. For example, Prognosis A 313 is the result of the presence of certain biomarkers along with the headache rated 8-10 (symptom 345) at evaluation time. Based on these results, the Prognosis A 313 for the patient is that they will develop moderate to severe depressive symptoms at 1 month post-TBI.

Similarly, prognosis B 323 can be made based on the biomarker results 353, symptoms at evaluation time 355, and other inputs to the diagnosis and prognosis application 134, including PHI, and other metrics or physiological measures. The diagnosis and prognosis application 134 can create a chart of symptoms and the relative risk of the patient developing that symptom, such as the simplified example table 285 in FIG. 2.

### Determining Severity of Brain Injury

Exemplary embodiments of the invention provide methods for determining the severity of brain injury in a patient. Each grade or stage of brain injury likely has a characteristic level of a biomarker or relative levels/ratios of a set of biomarkers (a pattern or ratio). The severity of brain injury is determined by measuring the relevant biomarkers and then either submitting them to a classification algorithm or comparing them with a reference amount, i.e., a predefined level or pattern of biomarkers that is associated with the particular stage.

### Determining Brain Injury Prognosis

Exemplary embodiments of the invention provide methods for determining the course of brain injury in a patient. Brain injury course refers to changes in brain injury status over time, including brain injury progression (worsening) and brain injury regression (improvement). Over time, the amount or relative amount (e.g., the pattern or ratio) of the biomarkers changes. For example, biomarker “X” may be increased with brain injury, while biomarker “Y” may be decreased with brain injury. Therefore, the trend of these biomarkers, either increased or decreased over time toward brain injury or recovery, indicates the course of the condition. Accordingly, this method involves measuring the level of one or more biomarkers in a patient at least two different time points, e.g., a first time and a second time, and comparing the change, if any. The course of brain injury is determined based on these comparisons.

### Patient Management

In certain embodiments of the methods of qualifying brain injury status, the methods further comprise managing patient treatment based on the status. Such management includes the actions of the physician or clinician subsequent to determining brain injury status. For example, if a physician makes a diagnosis of brain injury, then a certain regime of monitoring would follow. An assessment of the course of brain injury using the exemplary methods of the invention may then require a certain therapy regimen. Alternatively, a diagnosis of no brain injury might be followed with further testing. Also, further tests may be called for if the diagnostic test gives an inconclusive result on brain injury status.

### Determining Therapeutic Efficacy of Pharmaceutical Drug

Exemplary embodiments of the invention provide methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern, profile or ratio) of one or more of the biomarkers may change toward a brain injury status profile. Therefore, one can follow the course of one or more biomarkers in the patient during the course of treatment. Accordingly, this method involves measuring one or more biomarkers in a patient receiving drug therapy, and correlating the biomarker levels/ratios with the brain injury status of the patient (e.g., by comparison to predefined levels/ratios of the biomarkers that correspond to different brain injury statuses). One embodiment of this method involves determining the levels/ratios of one or more biomarkers for at least two different time points during a course of drug therapy, e.g., at a first time point and at a second time point, and comparing the change(s) in levels/ratios of the biomarkers, if any. For example, the levels/ratios of one or more biomarkers can be measured before and after drug administration or at two different time points during drug administration. The effect of therapy is determined based on these comparisons. Accordingly, the effectiveness of a patient's treatment or therapy can be monitored over time. If a treatment is effective, then the level/ratio of one or more biomarkers will trend toward normal, while if treatment is ineffective, the level/ratio of one or more biomarkers will trend toward a particular brain injury status.

### Generation of Classification Algorithms for Qualifying Brain Injury Status

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., brain injury versus no brain injury).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Patent Application Publication No. 2002/0138208 A1 to Paulse et al., “Method for analyzing mass spectra,” which is incorporated by reference in its entirety.

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application Publication No. 2002/0193950 (Gavin et al. “Method or analyzing mass spectra”), U.S. Patent Application Publication No. 2003/0004402 (Hitt et al., “Process for discriminating between biological states based on hidden patterns from biological data”), and U.S. Patent Application Publication No. 2003/0055615 (Zhang and Zhang, “Systems and methods for processing biological expression data”). These publications are incorporated by reference in their entireties.

The classification models can be formed on and used on any suitable digital computer. Suitable digital computers include micro, mini, or large computers using any standard or specialized operating system, such as a UNIX, WINDOWS® or LINUX™ based operating system. In embodiments utilizing a mass spectrometer, the digital computer that is used may be physically separate from the mass spectrometer that is used to create the spectra of interest, or it may be coupled to the mass spectrometer.

The training data set and the classification models according to exemplary embodiments of the invention can be embodied by computer code that is executed or used by a digital computer. The computer code can be stored on any suitable computer readable media including optical or magnetic disks, sticks, tapes, etc., and can be written in any suitable computer programming language including R, C, C++, visual basic, etc.

The learning algorithms described above are useful both for developing classification algorithms for the biomarkers already discovered, and for finding new biomarkers. The classification algorithms, in turn, form the base for diagnostic tests by providing diagnostic values (e.g., cut-off points) for biomarkers used singly or in combination.

### Kits for the Detection of Biomarkers

In another aspect, exemplary embodiments of the invention provide kits for qualifying brain injury status, which kits are used to detect the biomarkers described herein. In a specific embodiment, the kit is provided as an enzyme linked immunosorbent assay (ELISA) kit comprising antibodies to biomarkers including, but not limited to one of more of the biomarkers disclosed herein

The ELISA kit may comprise a solid support, such as a chip, microtiter plate (e.g., a 96-well plate), bead, or resin having biomarker capture reagents attached thereon. The kit may further comprise a means for detecting the biomarkers, such as antibodies, and a secondary antibody-signal complex, such as horseradish peroxidase (HRP)-conjugated goat anti-rabbit IgG antibody or tetramethyl benzidine (TMB) as a substrate for HRP.

The kit may be provided as an immuno-chromatography strip comprising a membrane on which the antibodies are immobilized, and a means for detecting, e.g., gold particle bound antibodies, in which the membrane may be a nitrocellulose-based (NC) membrane, a PVDF membrane, or other suitable type of membrane used in the art. The kit may comprise a plastic plate or substrate onto which a sample is applied and immobilized detection agents, such as detectably labeled antibodies, e.g., gold particle-bound antibodies temporally spaced and immobilized on the substrate, e.g., a glass fiber filter or a nitrocellulose membrane comprising one or more bound antibodies (immobilized in one or more bands on the substrate), and a bound secondary antibody (immobilized in an band on the substrate) and an absorbent pad are positioned in a serial manner, so as to keep continuous capillary flow of blood or serum over the immobilized detection reagents.

In certain embodiments, a patient can be diagnosed by adding a biological sample (e.g., blood or serum) from a patient to the kit, or components thereof, and detecting the relevant biomarkers using antibodies that specifically bind to the biomarkers. By way of example, the method comprises: (i) collecting blood from the patient; (ii) adding the blood or serum from the patient to the components in the kit, e.g., a holding tube or a substrate; and (iii) detecting the biomarkers to which the antibodies have bound. In this method, the antibodies are brought into contact with the patient's blood. If the biomarkers are present in the sample, the antibodies will bind to the sample, or a portion thereof. In other kit and diagnostic embodiments, blood is not collected from the patient (i.e., it is already collected), and is assayed for the presence of biomarkers using the kit. Moreover, in other embodiments, the sample may comprise a tissue sample or a clinical sample, which can be processed, e.g., homogenized and/or suspended in medium or buffer, prior to assay.

The kit can also comprise a washing solution or instructions for making a washing solution, in which the combination of the capture reagents and the washing solution allows capture of the biomarkers on the solid support for subsequent detection by, e.g., antibodies or mass spectrometry. In a further embodiment, a kit can comprise instructions for suitable operational parameters in the form of a label or separate insert. For example, the instructions may inform a consumer or user about how to collect the sample, how to wash the probe or the particular biomarkers to be detected, etc. In yet another embodiment, the kit can comprise one or more containers with biomarker samples, to be used as standard(s) for calibration or normalization.

The practice of the exemplary embodiments of the invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These publications are incorporated by reference in their entireties. These techniques are applicable to the production of the polynucleotides and polypeptides n, and, as such, may be considered in making and practicing exemplary embodiments of the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of Exemplary embodiments of the invention, and are not intended to limit the scope of what the inventors regard as their invention.

Without further elaboration, it is believed that one skilled in the art, using the preceding description, can utilize Exemplary embodiments of the invention to the fullest extent. The following examples are illustrative only, and not limiting of the remainder of the disclosure or claims in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Multiple Serum Biomarker Panels Identify Brain-Injured Patients in CT-Negative Populations

Head injury brings nearly 5 million patients into emergency departments per year in the US. Only a small percentage of these patients have a positive CT scan, showing structural evidence of injury. Adjunct diagnostic tests measuring changes in physiological levels of blood-borne biomarkers may therefore aid in identifying patients at risk for deleterious effects of head injury, and predict long-term consequences.

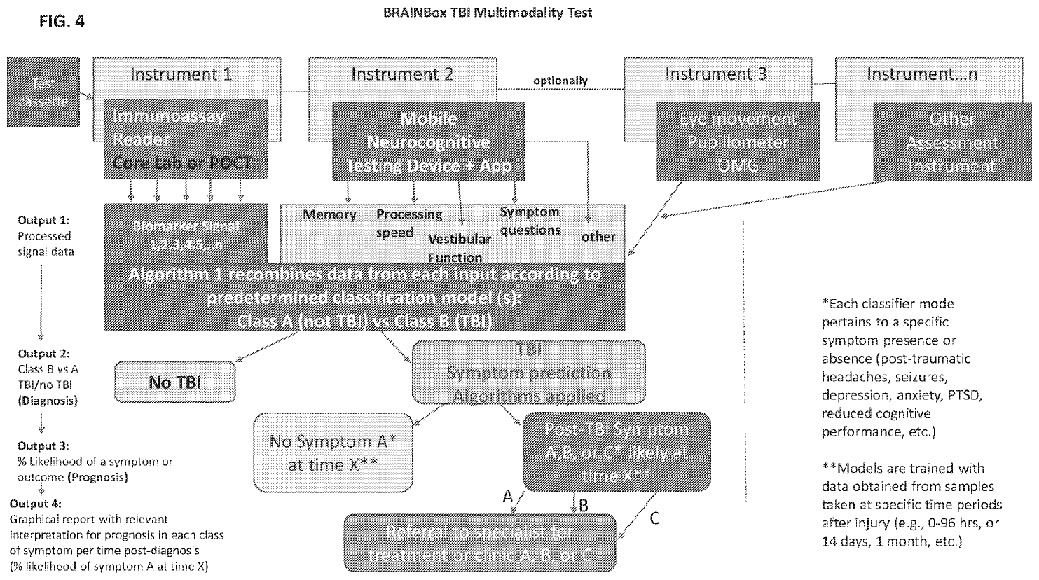

HeadSMART is a prospective study conducted at Johns Hopkins University, with serum sampling performed at initial evaluation, and at 7 subsequent time points up to 6 months post-injury. The current study was designed to evaluate the utility of 8 brain-specific protein biomarkers, namely, BDNF, GFAP, ICAM5, MT3, NRGN, citrullinated-NRGN, NSE, and/or SNCB, to diagnose brain injury. Biomarker assays were performed on a cohort of 200 brain-injured patients, and compared with 200 healthy control serum samples. Clinical data, with detailed neurocognitive and neuroimaging results were compiled, consistent with NIH common data elements (CDE). Table 2 herein shows the demographic breakdown of the HeadSMART study and acute symptoms used in diagnosis. Serum biomarker concentrations were assessed in replicate assays and the values obtained were subjected to machine learning trials. Three-analyte panels were able to classify patients as brain-injured in the CT negative population with high sensitivity and specificity (>0.95). These results indicate the potential utility of applying machine learning algorithms to serum biomarker findings in a point of care setting, in order to identify brain injury prior to CT, or to assess acute risk. (See FIGS. 1-11 of the Assignee's US Patent Application Publication US2019/0339291).

### Example 2: Serum Biomarker Panels Distinguish Between Severity and Location of Intracranial Hemorrhage

### Methods:

The study was approved by the Institutional Review Boards of the participating clinical unit; informed consent was obtained from all participants. In an initial study, sera derived from blood samples obtained from healthy subjects, and from subjects suffering from brain injury at varying times post injury, and with varying clinical profiles, were tested using a sandwich ELISA-based microtiter multi-well plate assay with either colorimetric detection or electro-chemiluminescence detection methods (Meso Scale Discovery or “MSD”).

Blood samples and clinical data were collected from patients arriving at the emergency departments (ED) of Johns Hopkins Hospital (JHH, Baltimore; n=255). Defined human serum samples were used for this study. Samples from adult TBI patients were analyzed retrospectively. The control cohort of patients, evaluated for non-TBI complaints was obtained from Baylor College of Medicine (Houston, Tex.; n=250).

To be considered a TBI patient, the following criteria had to be met: 18 years old or greater, blunt TBI presenting within 24 hours of injury, met the American College of Emergency Physicians (ACEP) criteria for obtaining head CT scans in TBI. Patients having brain tumor, brain surgery, pregnant, non-English speakers, were excluded. Serial serum samples were collected from enrollment of up to 6 months from 255 TBI patients. Three samples per patient at three different time-points from injury were collected. For controls, 250 non-TBI individuals at least 18 years of age were recruited under informed consent. One blood sample was collected per control individual. All patient identifiers were kept confidential.

### Results:

Evaluation of Six brain-specific protein biomarkers to diagnose brain injury (BDNF, GFAP, MT3, NRGN, NSE and SNCB) identified three-analyte panels that performed with >95% sensitivity and specificity to identify ACRM+ TBI samples versus healthy controls. Single and multi-analyte panels were compared for their ability able to classify patients according to specific CT findings, including severity of hemorrhage and evidence of intra-parenchymal hemorrhage. These findings include that individual markers such as Neurogranin (NRGN) can identify patients with intra-parenchymal bleeding (ROC, Sensitivity >0.9, specificity 0.625). See FIG. 1A of the Assignee's US Patent Application Publication US2019/0339291) and that the severity of hemorrhage could be differentiated with small panels (e.g., SNCB, NRGN, GFAP, Sensitivity 0.864, Specificity 0.625) versus brain-injured in the CT negative population. These results, specific to subcategories of neuroimaging findings, may assist in guiding patient care and indicate the potential utility of applying machine learning algorithms to serum biomarker findings in a point of care setting, in order to identify specific brain injury features prior to CT, or to assess acute risk.

### Example 3: Use of Biomarker Values to Predict Patient Recovery Post-Injury

Measurement of serum biomarker levels with MSD or ELISA assays was analyzed in groups of patients with single or combinations of neurological or neuropsychiatric clinical data (symptoms at initial evaluation in the emergency medicine setting), and tested for their ability to discriminate between different global disability and recovery outcomes such as Glasgow Outcome Score Extended (GOSE), and Post-Concussive Syndrome (PCS), measured at 1, 3, and 6 months post-injury. The differences in median values between outcome classes for GOSE (7-8 being lower and upper complete favorable recovery, and GOSE 1-6 being poor recovery) were determined using Wilcoxon Rank Sum Test. The significant differences were determined using a 95% confidence threshold. (See FIGS. 12-17 of the Assignee's US Patent Application Publication US2019/0339291.)

A combination of clinical data and objective biomarker levels was used to predict outcomes, and could serve as the basis for a return to work or return to play test, wherein the test determines whether an individual fitting a symptom group has returned to biomarker levels that predict a favorable outcome or absence of disease.

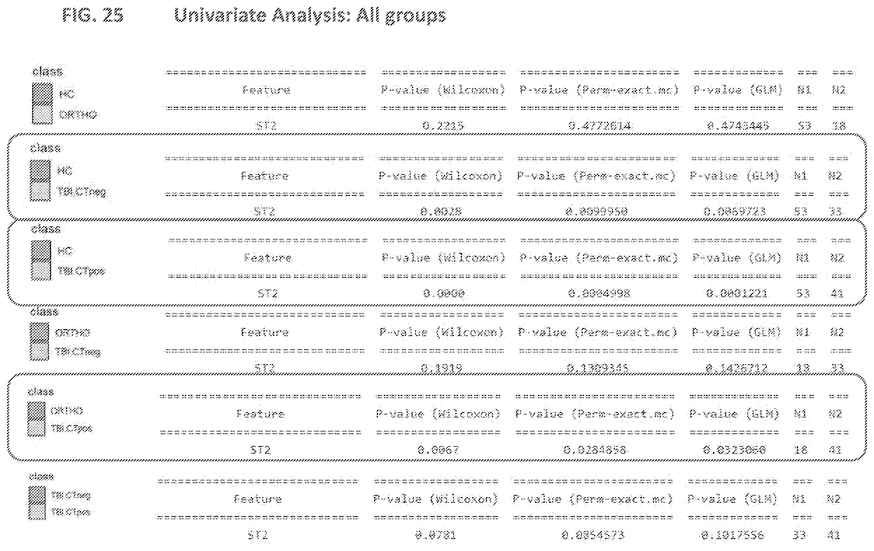

By way of example, the table presented in FIG. 25 of the Assignee's US Patent Application Publication US2019/0339291) reflects the use of biomarker values to predict patient recovery at 1-month post injury. The table in FIG. 25 shows data based on CT negative (CT−) patients enrolled in the HeadSMART study. The HeadSMART (Head Injury Serum Markers for Assessing Response to Trauma study (HeadSMART)) aims to examine blood-based biomarkers for diagnosing and determining prognosis in traumatic brain injury (TBI). HeadSMART is a 6-month prospective cohort study comparing emergency department patients evaluated for TBI (exposure group) to (1) emergency department patients evaluated for traumatic injury without head trauma and (2) healthy persons. Study methods and characteristics of the first 300 exposure participants are discussed in Peters, M. E. et al., 2017, Brain Inj., 31(3):370-378. As reported by Peters et al., of the first 300 participants in the exposure arm, 70% met the American Congress of Rehabilitation Medicine (ACRM) criteria for TBI, with the majority (80.1%) classified as mild TBI. The majority of subjects in the exposure arm had Glasgow Coma Scale scores of 13-15 (98.0%), normal head computed tomography (81.3%) and no prior history of concussion (71.7%).

To obtain the data presented in the FIG. 25 table of the Assignee's US Patent Application Publication US2019/0339291) four different methods of assessing patient outcomes at 1 month after injury were applied: GOSE, ICD10-PCS (The International Classification of Diseases, 10^{th} Revision, Procedure Coding System), GOSE or ICD10-PCS, and GOSE and ICD10-PCS, which are defined in the figure below the table of the Assignee's US Patent Application Publication US2019/0339291 as follows: ICD10-PCS: an ICD10-based post concussive symptom score (Scored as 0=no PCS, 1=mild PCS; 2=moderate to severe PCS); and GOSE (Glasgow Outcome Scale-Extended (Scored 1-8, with 8 being complete recovery). For each of the four assessment methods, data are presented for the 10 best performing panels of biomarkers. The biomarker panels include SNCB. In the 1-month patient outcome assessment shown in FIG. 25 of the Assignee's US Patent Application Publication US2019/0339291), biomarker levels were used along with other factors (e.g., depression, headache/severe headache, amnesia, gender, age). The data can be used to select the biomarker panels that best discriminate and stratify risk.

### Example 4: Two Serum Biomarkers Identify Sustained Injury in Mild TBI Cohorts and American Football Players

The current study was designed to evaluate the utility of brain-specific protein biomarkers detectable in human serum to diagnose brain injury in suspected concussed or mild TBI patients.

Methods: Highly sensitivity ELISA assays (traditional ELISA or MSD) were developed to detect Neurogranin (NRGN) and Synuclein Beta (SNCB). Serum biomarker concentrations were assessed in replicate assays. Markers were used to study two separate clinical studies, one mild TBI and the second a cohort of football players sampled prior to and during the football season. The cohort of mild TBI from HeadSMART (n=192 Johns Hopkins University, 2 sites), was compared with healthy control serum samples (n=250, Baylor College of Medicine). The football players (n=25 off season, n=25 on season), were obtained from Ben Gurion University of the Negev (Age range 18-39, median 25.5; and compared with 52 age-matched healthy controls, age range 18-39, median 28). Results from serum biomarkers were tested in traditional logistic regression, and in machine learning algorithms (including FLDA and LogitBoost).

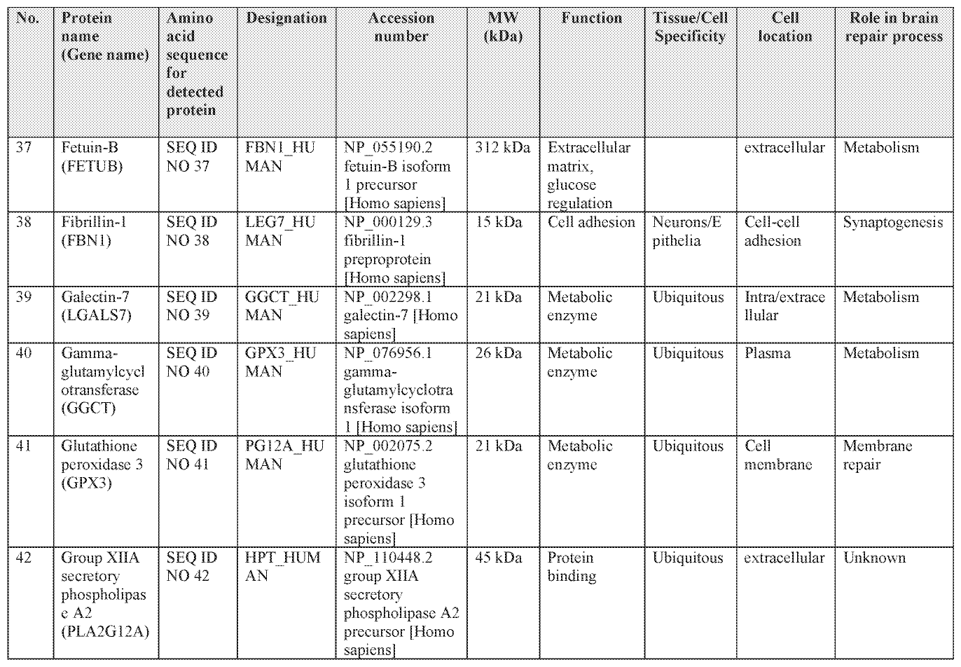

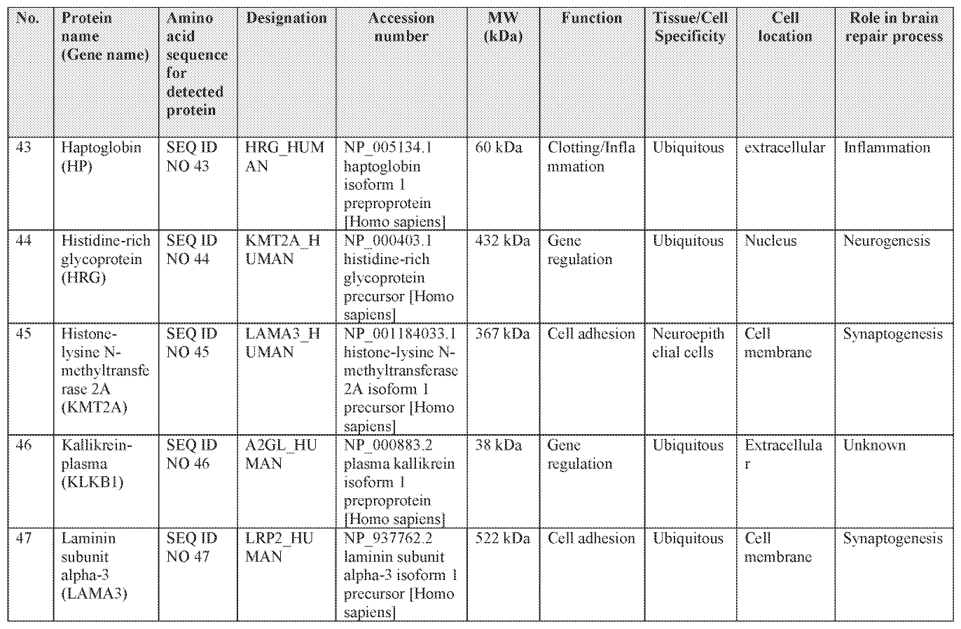

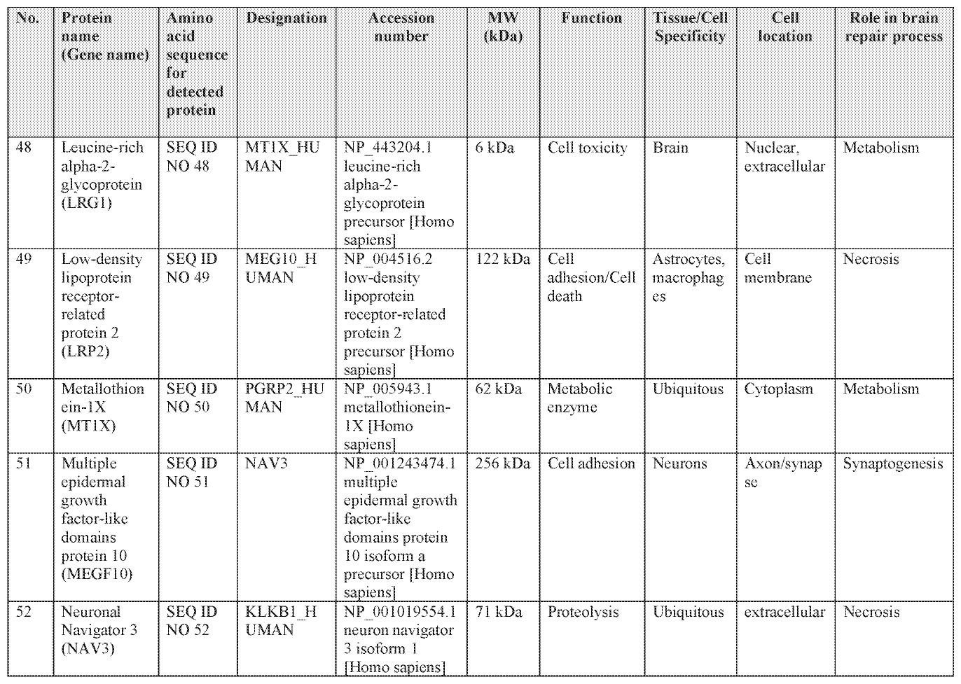

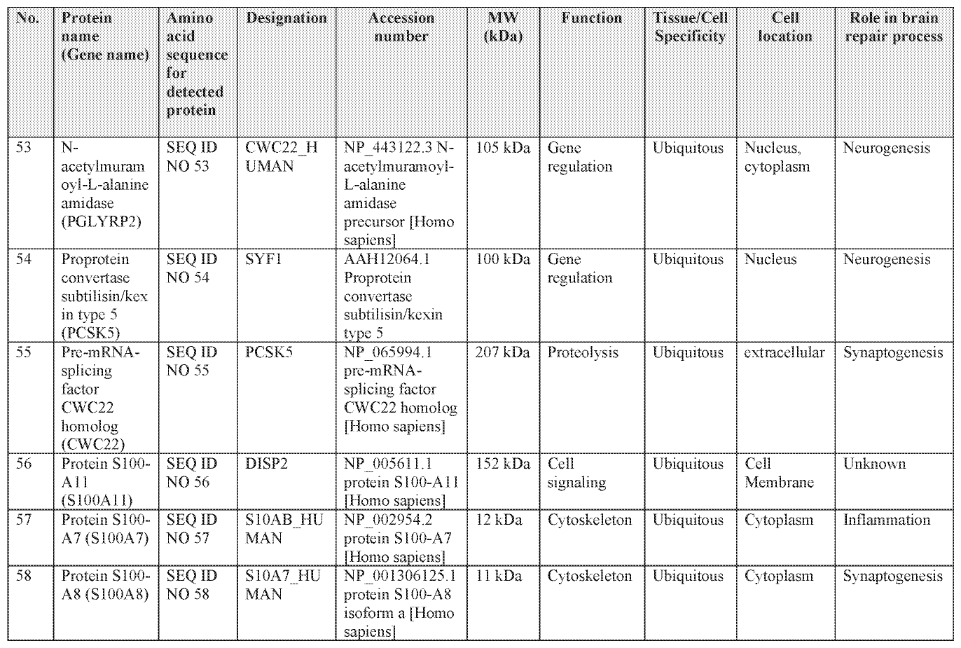

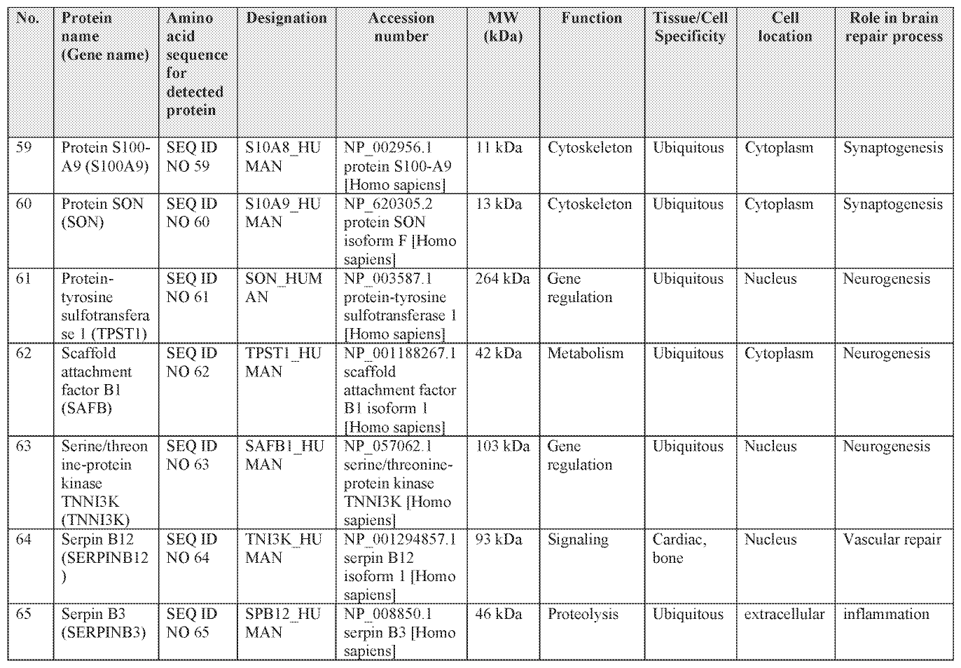

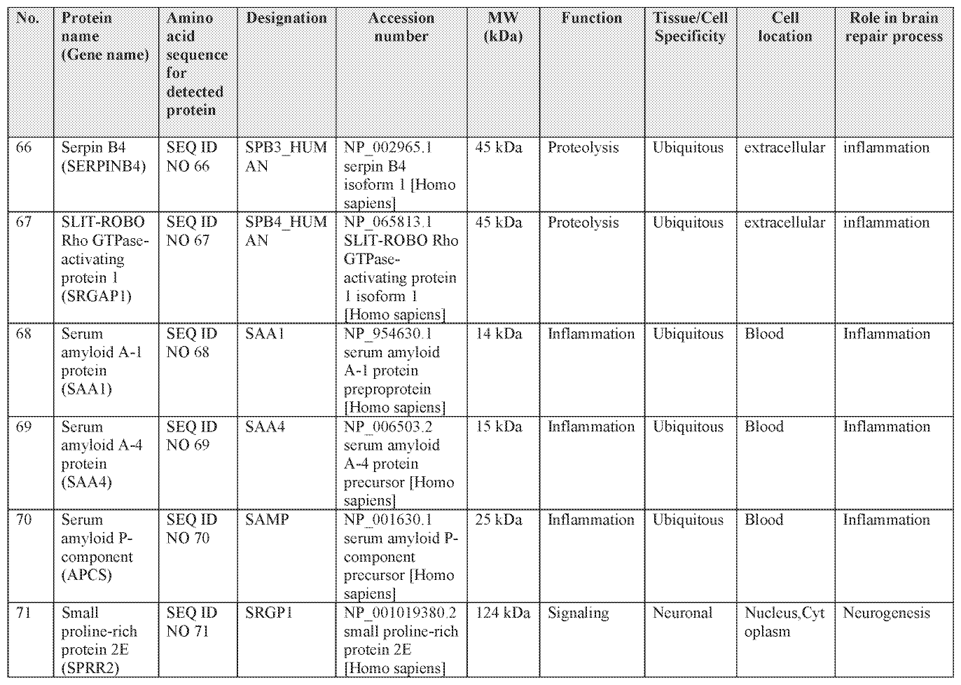

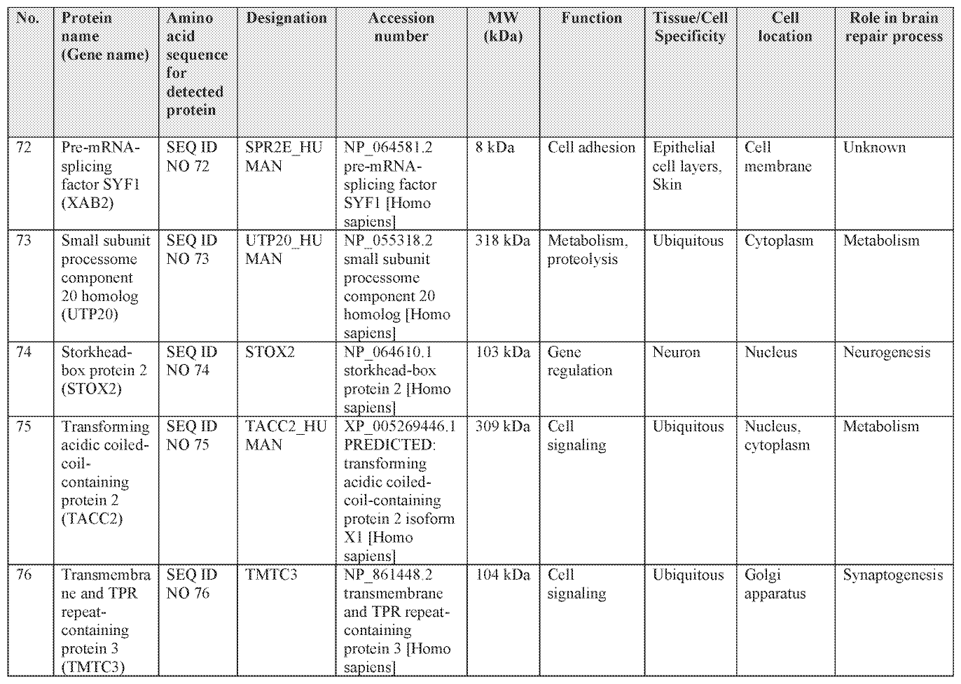

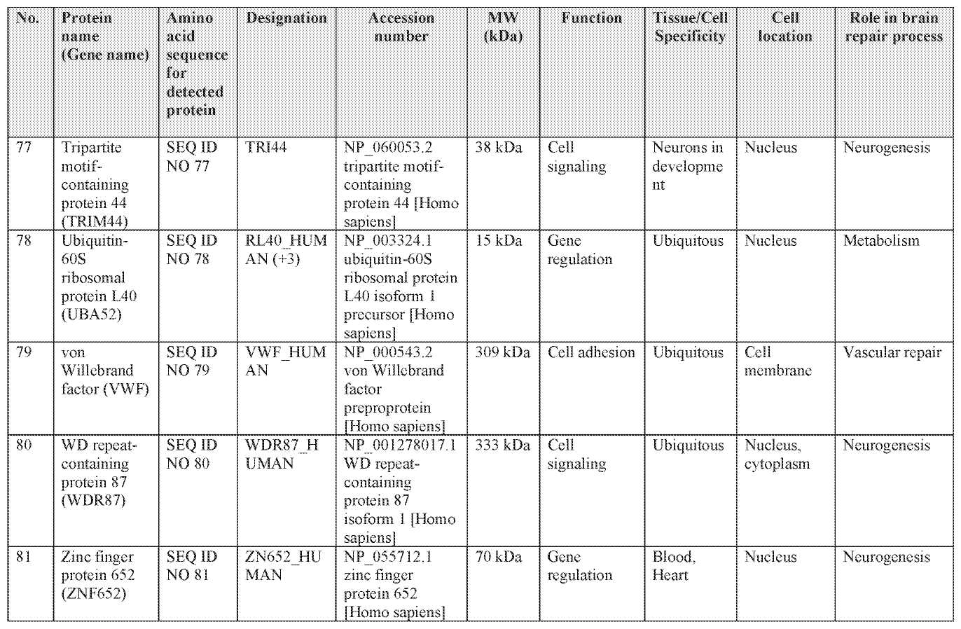

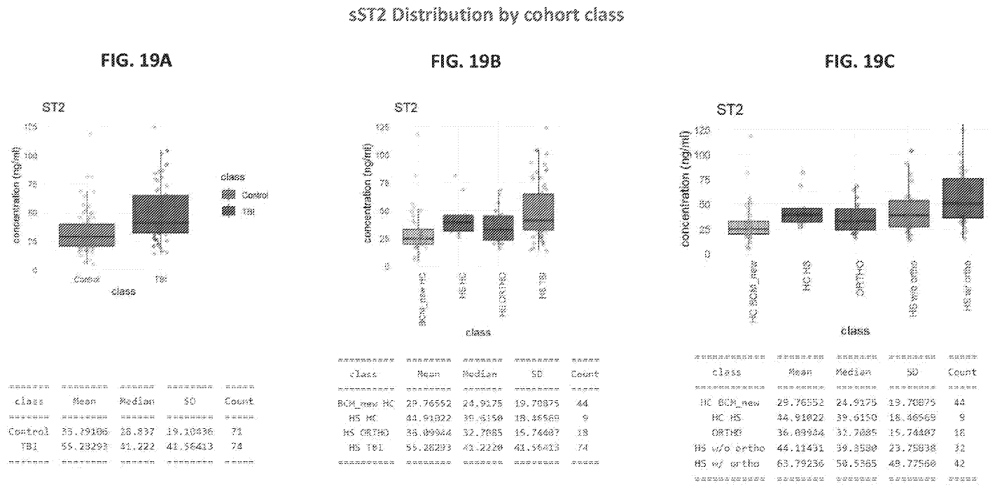

Results: NRGN and SNCB were individually able to classify patients as brain-injured compared to control in the HeadSMART cohort. Additionally, football players were distinguished from controls for both markers studied (FIGS. 18 and 19 of the Assignee's US Patent Application Publication US2019/0339291)). Median SNCB levels significantly differed in players between the off and on season (p=0.0014; Wilcoxon Rank Sum Test). Receiver Operator Curve analysis demonstrated areas under the curve (AUCs) of greater than 0.95 for Neurogranin in differentiating healthy controls from football players during either the on or off season, with improved sensitivity and specificity when both markers were used in a panel (Sensitivity 96%, specificity 61% for control vs. off season; Sensitivity 96%, specificity 65% for control vs. on season).

Conclusions: The use of serum biomarker proteins Neurogranin and SNCB to detect injury in mild TBI patients and football players who have sustained injury may provide useful information to direct post-injury care and inform return to work and play decisions. Refined application of machine learning algorithms to answer specific clinical questions is a useful tool that can inform treatment decisions.

### Example 5: Biomarker Panels Useful in Distinguishing Mild TBI (mTBI)

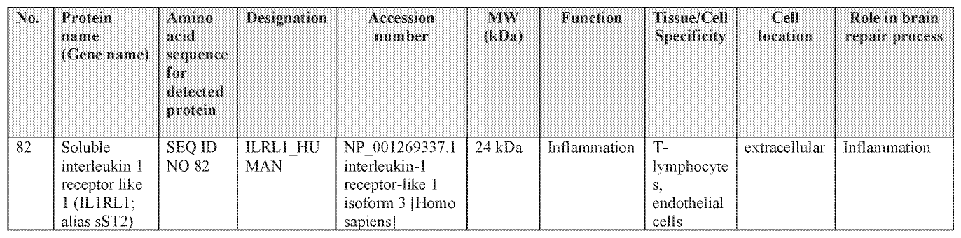

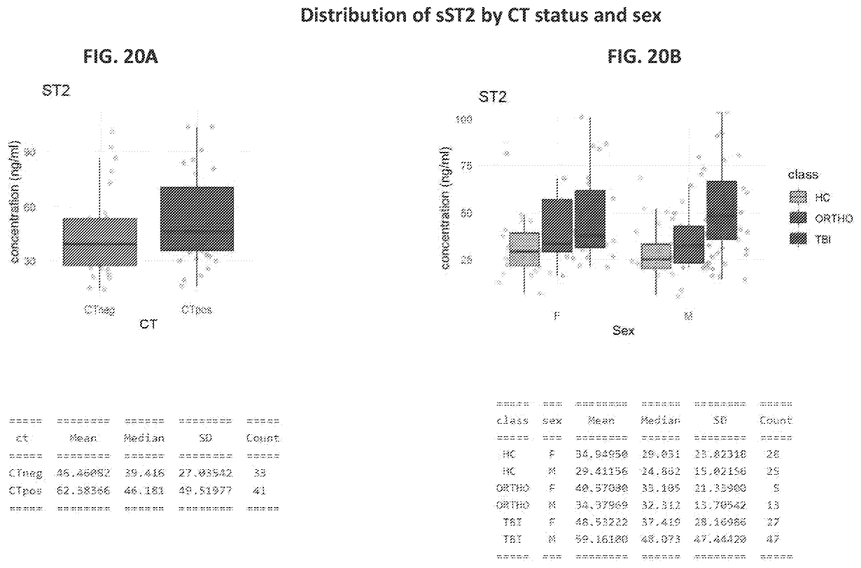

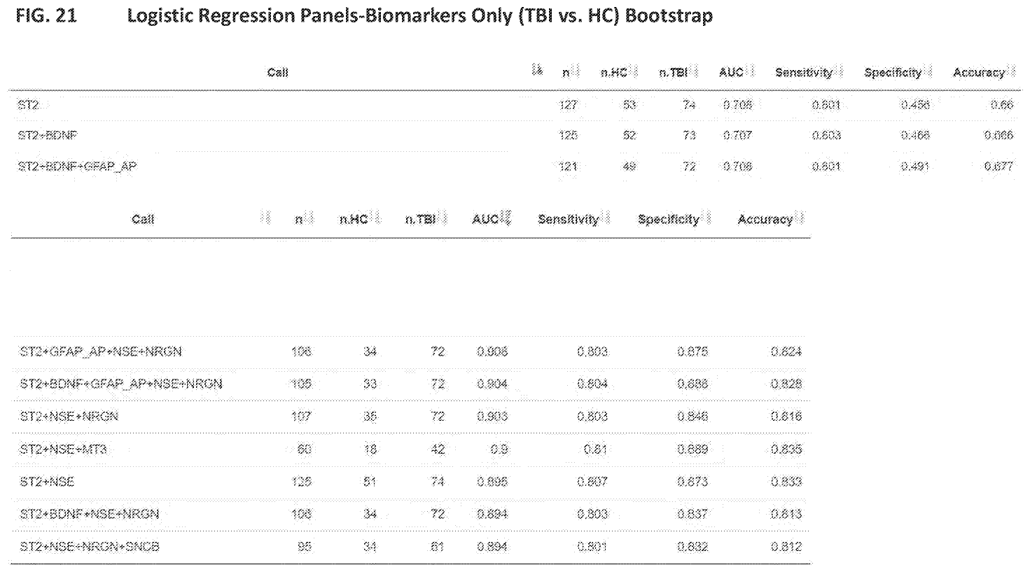

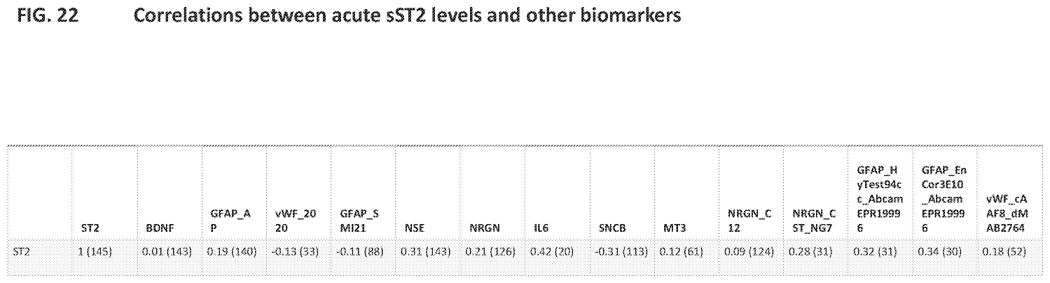

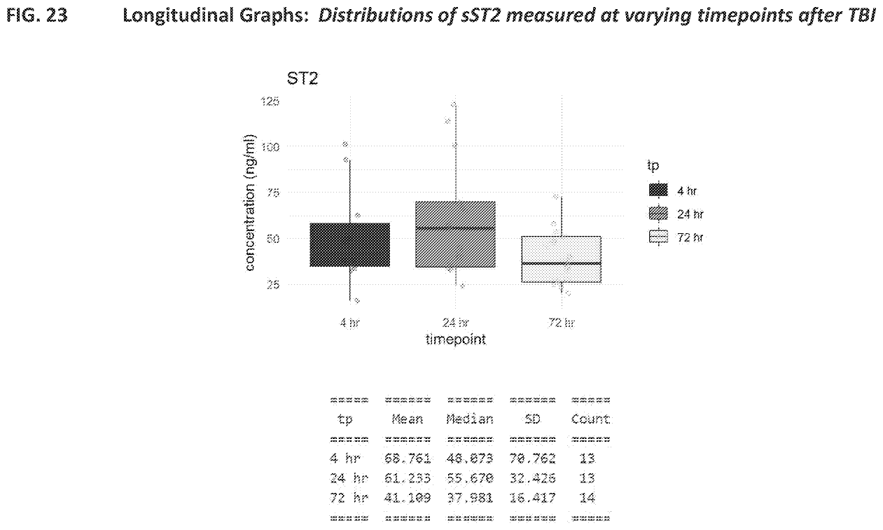

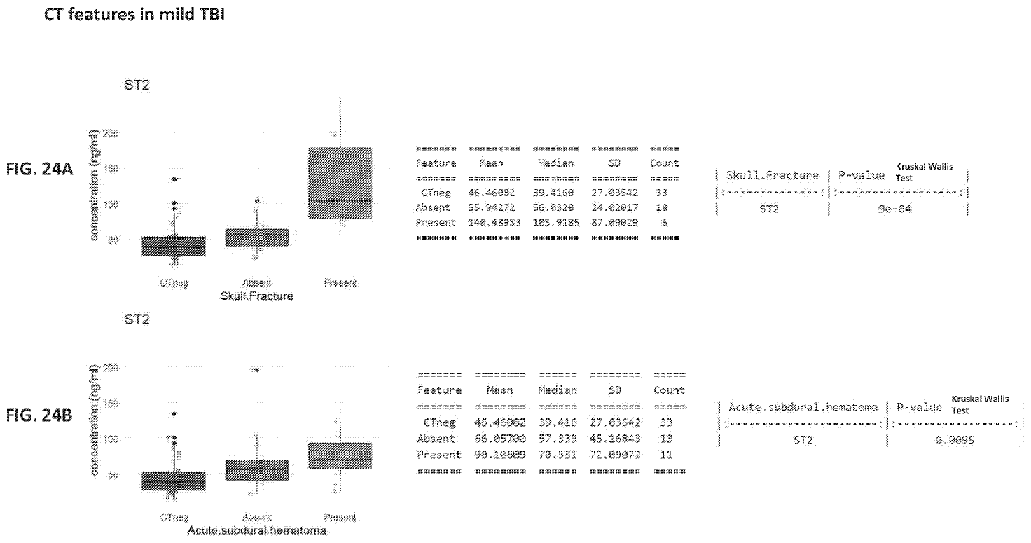

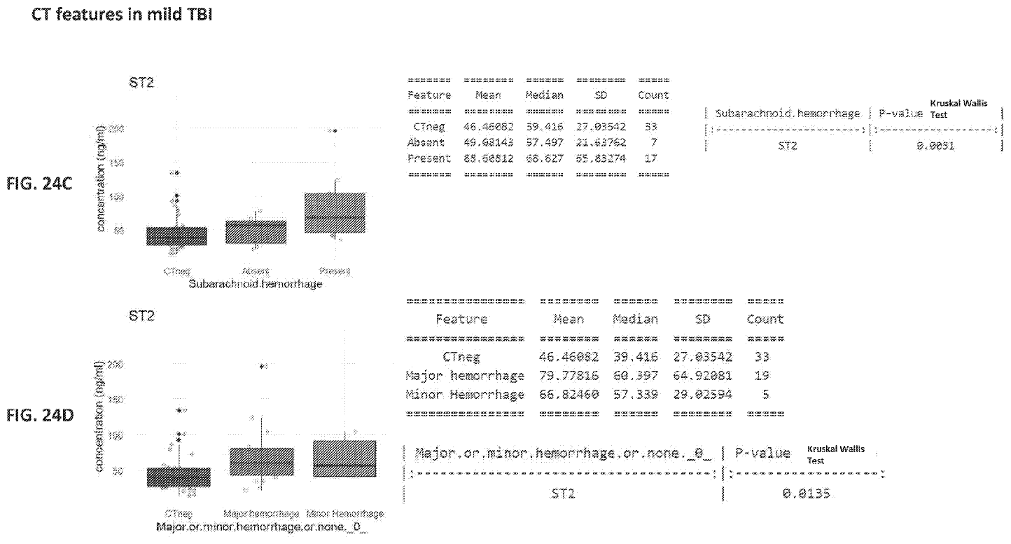

Biomarkers were assayed using a multi-array technology that combines Electrochemiluminescence and arrays, which is available from MesoScale Discovery (MSD). The MSD ELISA assay found that levels of NRGN, NSE, GFAP and ICAM5 were increased in patient serum, and that levels of MT3 and SNCB were decreased in patients with mild brain injury as determined by physician assessment using the American Congress of Rehabilitation Medicine (ACRM), (FIG. 20 of the Assignee's US Patent Application Publication US2019/0339291)). Traumatic brain injury could be distinguished on the basis of alterations in biomarker levels. The specificity and sensitivity of diagnosis was increased by the use of multiple biomarkers (FIG. 21 of the Assignee's US Patent Application Publication US2019/0339291)). Machine learning algorithms were also used to improve performance of biomarker panels (FIG. 22 of the Assignee's US Patent Application Publication US2019/0339291)). A four biomarker panel including NRGN, SNCB, MT3 and ICAM5 was used as a classifier for distinguishing mild TBI (mTBI) (ACRM+, n=334) from healthy controls (n=268). The performance of the classifier was assessed with cross-validation using the HeadSMART TBI study and controls. The machine learning algorithm showed better diagnostic performance. Interestingly, the inclusion of patient age and sex in the models improved both diagnostic performance and specificity (FIG. 22 of the Assignee's US Patent Application Publication US2019/0339291)). Current longitudinal linear mixed effects models were developed using serial sampling of 500 HeadSMART mild TBI patients and complete clinical information, developed on longitudinal biomarker level measurement by MSD-ELISA, which was performed for 8 time point blood draws per patient, over a 6 month recovery period. Each model type (longitudinal predictive modeling vs. machine learning classifier) also supported the use of multi-analyte panels for increased prediction accuracy (FIG. 23 of the Assignee's US Patent Application Publication US2019/0339291)). Models built with mTBI patients from the HeadSMART study were used to predict patient outcomes using the Extended Glasgow Outcome Scale (GOS-E). Adjusting for other clinical covariates (i.e., gender, age, and race) was found to be helpful for the best predictive model with some biomarkers. Prediction accuracy was determined by testing models on an independent TBI cohort. These results suggest that use of multiple markers and multiple time points both improve prediction of outcomes (FIG. 24 of the Assignee's US Patent Application Publication US2019/0339291)).

### Example 6: Machine Learning Models Identify Mild TBI (mTBI) Patients with Significant Depressive Symptoms at 1, 3 and 6 Months Using Three Serum Biomarkers

Head injury brings nearly 5 million patients into emergency departments (ED) per year in the US. While many receive a CT scan, only a small percentage of patients show structural evidence of injury. This Example describes the identification of serum biomarkers that objectively classified patients with traumatic brain injury (TBI) who are at risk for chronic neuropsychiatric sequelae. The HeadSMART prospective study was conducted at Johns Hopkins University School of Medicine, enrolling patients with traumatic brain injury at two separate hospitals in Baltimore, Md. A total of 500 brain-injured patients aged 18-80 were tested for the presence of serum biomarkers within the first 24 hours after injury (Mean 5.25 hours). As described hereinabove, it was demonstrated that panels of three biomarkers can identify patients with TBI, using objective blood tests, by applying machine learning algorithms such as random forest. In the study described in this example, a more comprehensive analysis of a larger number of machine learning algorithms was conducted using data from five serum biomarkers: Brain-Derived Neurotrophic Factor (BDNF), Glial Fibrillary Acidic Protein (GFAP), Neurogranin (NRGN), Neuron Specific Enolase (NSE) and Synuclein Beta (SNCB). These five biomarker proteins were tested for in all subjects using colorimetric or electroluminescence-based sandwich ELISA assays.

Clinical assessment employing the Patient Health Questionnaire 9 (PHQ9) was performed at 1, 3, and 6 months post injury. Moderate to severe depressive symptoms were equivalent to a score of 10 or greater in this assessment. Models utilized only patients without history of seizures, prior TBI, or neurological disease and who presented with severe headache (total n=106). The analysis was performed in R using classification algorithms implemented in the Caret package from the following categories: generalized linear models, discriminant analysis models, Bayesian models, bagging, boosting and ensemble models. Models of three marker panels were built using 5-fold cross validation repeated 5 times in each algorithm. The models were compared through ROC analysis, considering only those that provided AUCs>0.7. To adjust for any imbalances in age and sex between the groups, the models included age and sex of the patients. At three months post injury, eXtreme Gradient Boosting yielded the best class prediction for mild TBI patients sustaining moderate to severe depressive symptoms using the acute marker panel NRGN, GFAP, and NSE (AUC=0.76; sens=0.80, spec=0.54, number of samples analyzed 67).

The panel of markers containing BDNF, GFAP, and NSE yielded the highest AUCs for prediction of depression in patients with PHQ9<10 at 1 or 3 months and at 6 months post-injury (AUC=0.72; sens=0.81, spec=0.47, number of samples analyzed 63). These methods form the basis of testing panels for objectively identifying patients with TBI, and for predicting which individuals will suffer from chronic depressive symptoms during the recovery period. Such tools can assist medical personnel in recommending therapeutic interventions, and can be used in clinical trials designed to assess the efficacy of preventive treatments to ameliorate depressive symptoms following TBI.

### Example 7: Predicting Incomplete Recovery Using Machine Learning: Determining Top-Performing Algorithms for Identifying CT Negative, Mild TBI Patients Who Will have Poor Recovery

This example describes a study conducted to identify small panels of serum biomarkers that objectively identify patients with traumatic brain injury who will have good overall recovery. The HeadSMART prospective study was conducted at Johns Hopkins University School of Medicine, enrolling 500 brain injured patients. Patients aged 18-80 were tested for serum levels of Brain-Derived Neurotrophic Factor (BDNF), Glial Fibrillary Acidic Protein (GFAP), Neurogranin (NRGN), Neuron Specific Enolase (NSE) and Synuclein Beta (SNCB) in blood samples collected within the first 24 hours after injury (median 4.2 hours; average 5.25 hours post-injury). This study tested machine learning algorithms appropriate to the nature of the data. Sandwich ELISA assays were performed as single marker assays for all patients, and the averaged serum protein concentrations were used, along with outcome assessments, to build predictive models. Patients who had no previous history of neurological disease, who had no previous concussion and who reported severe headache as a symptom were included in the models (total n=106). Clinical assessment of patient outcomes included GOS-E (overall functional recovery) and ICD10-based post-concussive syndrome scoring (ICD10-PCS, symptom-based recovery score), assessed at three time points: 1, 3, and 6 months post-injury. For GOS-E, a score of 7 or 8 was considered good recovery. ICD10-PCS greater than zero was considered incomplete recovery (i.e., score of 1 for mild ICD10-PCS and 2 for moderate to severe ICD10-PCS). Analysis was performed in R by ROC analysis using generalized linear models, discriminant analysis models, Bayesian models, bagging, boosting and ensemble classification algorithms from the Caret package. Models of three marker panels were built using 5-fold cross validation repeated 5 times in each algorithm. The models were compared in ROC analysis. To adjust for any imbalances in age and sex between the groups, the models were also built using biomarkers with patient age and sex included, and the results were compared.

At one month post-injury, delayed functional or symptom recovery predictions could be best assessed by random forest (Best AUC=0.74; sens=0.81, spec=0.43, with number of samples analyzed=63) using a combined outcome measure where GOS-E and ICD10-PCS scores were both required to be optimal for recovery (GOS-E=7, 8 or ICD10-PCS=0). The best performance across several algorithms was obtained using the three marker panel NRGN, BDNF, and SNCB. The prediction of outcomes for the other time points after injury required a different set of biomarkers for the optimal performance. Combined outcome measures performed better than individual metrics (e.g., GOS-E score alone).

This comparative study showed that objective prediction of adverse 1 month outcomes could be achieved using a number of machine learning models in CT negative, mild TBI patients whose symptomology may be otherwise unclear. These tests can be used in the field or in any other acute setting to determine which individuals will have delayed recovery and be in need of further interventions and testing.

### Example 8: Determination of a Three-Biomarker Panel to Improve Diagnosis in Patients with Mild Traumatic Brain Injury

Of nearly 5 million annual US emergency department (ED) visits for traumatic brain injury (TBI), fewer than 10% have computed tomographic (CT) evidence of abnormality. Despite no CT evidence in most patients who visit the ED, some suffer protracted somatic, behavioral, and neurocognitive dysfunction. This example describes a study to identify a biomarker panel that could diagnose traumatic brain injury (TBI) and long term effects in CT negative patients.

A prospective observational study of ED head injured patients versus healthy volunteers was performed. All patients were 18-80 years old and provided informed consent. Head injured patients had both a Glasgow Coma Score 13 and a head CT obtained per Canadian Head CT Rule criteria. TBI was defined by American Congress of Rehabilitation Medicine (ACRM) criteria. The biomarkers Neurogranin (NRGN), Neuron Specific Enolase (NSE) and Synuclein-Beta (SNCB) were evaluated in all patients within 24 hours of reported injury. Of 722 subjects studied, 268 were controls, and the TBI cohort (337 ACRM+, 117 ACRM −) median time from injury was 4.2 hours (IQR, 3.5; range 0.8-24 hours). The results showed that ACRM+ TBI patients had elevated NRGN and NSE, but decreased SNCB versus controls (p<0.001 for each). The highest C-statistic distinguishing ACRM+ versus controls was with a model using all 3 markers, age, and sex, and had a sensitivity and specificity of 98% and 77%, respectively. Marker panel positive ACRM negative patients had high 6 month rates of neuropsychiatric dysfunction. Use of a panel of NRGN, NSE and SNCB prospectively identified TBI in patients who also suffered high rates of adverse outcomes, despite initially being CT and/or ACRM negative. (FIG. 27 of the Assignee's US Patent Application Publication US2019/0339291)).

### Example 9

Since sST2 is a sensitive marker of acute inflammatory response to injury, retrospective human subject cohorts were tested for sST2 levels in blood samples using an FDA approved, colorimetric ELISA assay, and compared with luminescence-based sandwich immunoassay measurements of other brain injury biomarkers.

9.1 Discrimination of TBI from Control Subjects without Injury or with Orthopedic Injury.

Univariate analysis was used to assess sST2 as a discriminating biomarker. Box plots comparing the distributions of measured levels of sST2 in the healthy cohort (HC) and the orthopedic injury control cohort (ORTHO), or TBI subgroups (CT Neg or CT pos; or parsed by TBI—no other trauma—“HS without ortho”, or with other trauma—indicating polytrauma—“HS with Ortho”) are shown in FIGS. 6A-C. Orthopedic injury in the “HS with ortho” is synonymous with polytrauma, indicating the coincidence of any other significant extracranial injury in those patients.

FIG. 19A shows the distributions of sST2 in controls (combines 2 cohorts of healthy and one orthopedic injury control cohort) and mild TBI (all subgroups included together, separated by subgroups in the other plots). Number of subjects represented is included in the table directly below each plot.

FIG. 19B shows the distributions of 2 separate healthy control cohorts and an orthopedic injury cohort, compared with all HeadSMART mild TBI subjects examined.

FIG. 19C splits the cohorts further by separating TBI patients according to the presence or absence of polytrauma, which shows the further increase in median sST2 values when peripheral non-brain injuries are present. This is expected, since sST2 is a general inflammatory biomarker.

FIGS. 20A and 20B show the distributions of sST2 in serum in females vs male TBI subjects. The increase median value in CT positive (more severe) TBI injuries, which is further investigated by CT feature analysis in FIGS. 24A-D, described below. Males and females were not different between subgroups.

FIG. 25 shows the univariate analysis for all groups.

### 9.2 Biomarker Panels and Correlations

sST2 as an individual biomarker for distinguishing TBI patients from non-TBI controls was also tested by receiver operator curves (ROC curves) where the area under the curve (AUC), sensitivity, specificity, and accuracy were determined. sST2 was found to have good discriminative capability, but was a particularly strong contributor to the performance of biomarker combination panels for discriminating TBI from control subjects. The performance in combination with other biomarkers is shown for comparison. FIG. 21 provides logistic regressions for the identified biomarkers for the TBI versus HC cohorts. FIG. 22 shows the correlations between acute sST2 levels and other identified biomarkers.

Table 1A shows single and combination linear regression models show the increase in performance achieved by combining TBI biomarkers with sST2, compared to sST2 alone in discriminating TBI from controls based on the data shown in FIG. 21.

TABLE 1A

| Model | AUC | Sensitivity | Specificity | Accuracy |
|---|---|---|---|---|
| sST2 | 0.708 | 0.801 | 0.456 | 0.660 |
| sST2 + NSE | 0.895 | 0.807 | 0.873 | 0.833 |
| sST2 + NSE + NRGN | 0.903 | 0.803 | 0.846 | 0.816 |
| sST2 + NSE + MT3 | 0.900 | 0.810 | 0.889 | 0.835 |
| sST2 + NSE + NRGN + GFAP | 0.908 | 0.803 | 0.875 | 0.824 |

The correlations shown in FIG. 22 of biomarker values, as determined by mesoscale discovery electro-chemiluminescence assays (MSD-ELISA) (but any chemiluminescence-based sandwich or suitable immunoassays could be used), were examined using Spearman's correlation. The correlation with sST2 was determined for each of 14 immunoassays (from 8 total biomarker analytes since different versions of some assays were tested). Weak directional correlation was shown for IL-6, GFAP, and NSE and a weak negative correlation was shown for SNCB (colorimetric ELISA). The criterion used was correlation coefficient 0.25-0.50 was weak correlation. This level of correlation is interpreted to be due to injury severity correlations. Therefore, sST2 data provides unique information as an informative biomarker in TBI.

FIG. 23 compares models trained to distinguish mild TBI from healthy subjects (HC) showing longitudinal graphs of sST2 distributions measured at varying timepoints after TBI. Mild TBI samples tested over 3 days post-injury shown in FIG. 23 indicate a lower median level of sST2 at 72 hours compared to patients assessed at 24 hours or less. Median levels at 72 hours are equivalent to levels observed in trauma controls but have higher median value than healthy control subjects (compare with FIGS. 20A-B).

### 9.3 Discriminating Specific CT Features.

FIGS. 24A-D show models trained to distinguish TBI from a group of control subjects that includes both healthy controls and orthopedic injury, with no TBI (CONTROL). Table 1B summarizes the significant features discriminated by sST2 as a single biomarker clinically relevant mild TBI subgroups.

TABLE 1B

| | | Number | | FIG. |
|---|---|---|---|---|
| | | of | | with |
| Category | Feature | patients | P-value | data |
| Diagnosis | | | (Wilcoxon) | |
| of TBI+ | Healthy vs TBI (CTneg) | 53/33 | 0.0028 | 19C, 20B, |
| vs controls | Healthy vs TBI (CT Pos) | 53/41 | <0.0001 | 19C, 20B, |
| (TBI−) | Non-TBI trauma vs TBI | 18/41 | 0.0067 | 20B, |
| | (CT Pos) | | | |
| | CT neg vs CT Pos | 33/41 | 0.0781 | 19A, |
| CT | | | (Kruskal- | |
| Neuro- | | | Wallis*) | |
| imaging | Skull Fracture present | 51/6 | 0.0009 | 24A |
| | Subdural hemorrhage | 46/11 | 0.0135 | 24B |
| | Subarachnoid | 40/17 | 0.0031 | 24C |
| | hemorrhage | | | |
| | Major and minor | 33/24 | 0.0135 | 24D |
| | hemorrhage | | | |

*Kruskal Wallis test to compare medians between the distributions of subgroups where more than 2 groups were analyzed. In general, p values < 0.05 is a widely accepted threshold for demonstrating the difference.

Table 1B ranks the univariate analysis results on the basis of the p value obtained for the statistical test indicated. Comparison of the results of multiple tests, where used (Wilcoxon, Exact match permutation test, and generalized linear model; see FIG. 21), allows for greater confidence in the discriminatory performance for determining a difference between the subgroups.

### Methods

### Enrollment of Subjects

Patients included in this analysis were evaluated for TBI at the Johns Hopkins Hospital (Baltimore, Md.) and enrolled in the Head Injury Serum Markers for Assessing Response to Trauma (HeadSMART) study. Eligibility criteria included being 18-80 years of age, providing written informed consent, and having a Glasgow Coma Score (GCS) of 13-15. Patients in the TBI cohort received a standard of care head CT per the American College of Emergency Physicians (ACEP) criteria for TBI imaging, and were assessed by ACRM criteria. The control cohort was obtained at Baylor College of Medicine (Houston, Tex.), and consisted of non-patient ED waiting room volunteers enrolled after providing informed consent. Comprehensive health histories were taken to exclude head injury within 6 months, and patients had no known neurological disease, cancer or other major illness.

All TBI blood samples were obtained in the ED by dedicated research staff within 24 hours of injury. Serum (5 cc) and plasma (5 cc) from EDTA collection tubes (Becton Dickenson; Durham, N.C.) were obtained from both TBI and controls and were stored at −80° C.

### Biomarker Assays

Serum levels of Neurogranin (NRGN) and Neuron Specific Enolase (NSE) were tested using a sandwich immunoassay with electrochemiluminescence detection on a Quickplex 120 plate reader (Mesoscale Discovery; Rockville, Md.). Recombinant full length human NRGN and NSE proteins (Origene Technologies, Inc., Rockville, Md.) were used to generate a standard curve relating analyte concentration to luminescent signal. Mouse monoclonal capture antibodies and rabbit polyclonal antibodies were produced for NRGN (ImmunArray USA, Inc.; Richmond, Va.), or obtained from commercial sources for NSE (R&D Systems; Minneapolis, Minn.). Acceptance criteria included replicate samples varying less than 10% (CV), percent recovery 80-120% and regression curve linearity above 0.99. Synuclein Beta (SNCB) was tested for by peroxidase-mediated colorimetric ELISA with mouse monoclonal capture, biotinylated rabbit detection antibody, recombinant human SNCB protein standard, streptavidin-peroxidase conjugate, and tetramethylbenzidine (TMB) substrate, purchased from Fivephoton, Inc. (Fivephoton Biochemicals; San Diego, Calif.). Colorimetric detection for SNCB was performed on a Spectramax M3 microtiter plate reader (Molecular Devices, Inc., Sunnyvale, Calif.), by measuring absorbance of TMB substrate at 450 nm. The same assay QC criteria as above were also applied to assay results for SNCB.

### Clinical Outcomes

Clinical outcomes were evaluated by John Hopkins research staff at 1, 3 and 6 months post-TBI. Glasgow Outcome Scale-Extended (GOS-E) was used to determine global recovery status. ICD10 Post-Concussive Syndrome (ICD10PCS) scoring was used to evaluate global disability symptoms. Patient Health Questionnaire-9 (PHQ9) was used to provide an index of clinically significant depression. In the event that patients could not return for follow-up, interviews were accomplished by telephone. Follow-up outcomes were defined by a GOS-E score=8 as fully recovered. ICD10 post-concussive (PCS) symptoms were scored as 0 (healthy), 1 (mild PCS), and 2 (moderate/severe PCS). Finally, the PHQ9 score rated depressive symptoms as moderate/severe if a score was ≥10. (Korley, F. K. et al., 2016, J. Neurotrauma, 33(2):215-225).

### Statistical Analysis

Descriptive statistics were calculated for clinical features and biomarker data, assessing means and standard deviations for continuous variables, and counts and percentages for categorical variables. Biomarker values below the Lower Limit of Detection (LLOD) were substituted with a randomly generated number between zero and 0.5 times the LLOD for that biomarker assay, consistent with published standards (EPA QA/G-9). Transformation of the data using the natural logarithm was performed on all biomarker concentrations to reduce skewness in the distributions.

Performance of single and multi-marker combinations was compared using C-statistics (equivalent to the area under the ROC curve, AUC). For modeling, patients with missing biomarker data (samples not evaluated) were excluded. For each panel, a logistic regression model was fit and the C-statistic was estimated via stratified 10-fold cross-validation, which was subsequently repeated 5 times to reduce the variability of the estimates. (Kohavi, R., 1995, In Ijcai, Vol. 14(2):1137-1145; Kuhn, M. et al., 2013, Applied Predictive Modeling (Vol. 26), New York: Springer). Models were also constructed with a panel of all biomarkers using the random forest algorithm, and performance re assessed using stratified 10-fold cross-validation, repeated 5 times.

Clinical utility was also assessed by defining model performance cut points that provided a sensitivity of greater than 98% for an ACRM positive diagnosis. All data were analyzed by the statistical programming environment R version 3.3.0 and the integrated development environment for R, RStudio version 1.0.136. (RStudio Team, 2016, RStudio: Integrated Development for R, RStudio, Inc., Boston, Mass., URL. http://www.rstudio.com/).

### Results

Overall, 722 patients were enrolled. Of these, 454 were head-injured (337 ACRM positive, 117 ACRM negative) and compared to 268 healthy controls. While the sex distribution was similar across the entire population, there were more females (63.1%) in the control group, and more males (61.7%) in the head injured cohort. Demographics are reported in Table 2 below. The entire head injury cohort had a median time from injury to ED presentation of 4.2 hours (IQR, 3.5; range 0.8-24, hours). Stratifying by ACRM negative status identified a lower risk group, with lower rates of loss of consciousness (0 vs 76.3%), fewer positive CT scans (9.4% versus 20.5%), and higher rates of GCS=15 (100% vs 83.4%) for ACRM negative compared to ACRM positive patients, respectively.

TABLE 2
Demographics, Acute Clinical Symptoms and Mechanisms of Injury

| | | Head injured | | |
|---|---|---|---|---|
| | Control | ACRN− | ACRM+ | Total |
| | (n = 268) | (n = 117) | (n = 337) | (n = 454) |
| Mean Age, yrs. (SD) | 35.9 (±11.5) | 47.9 (±19.7) | 42.6 (±17.4) | 44.0 (±18.12) |
| Male (%) | 36.9% | 49.6% | 65.9% | 61.7% |
| Race | | | | |
| White | 26.9% | 56.4% | 47.8% | 50.0% |
| Black | 28.7% | 39.3% | 45.7% | 44.1% |
| Asia | 4.1% | 2.6% | 0.9% | 1.3% |
| Other (includes missing) | 40.3% | 1.7% | 5.6% | 4.6% |
| Ethnicity | | | | |
| Hispanic or Latino | 35.4% | 3.4% | 5.9% | 5.3% |
| Not Hispanic or Latino | 64.6% | 96.6% | 94.1% | 94.7% |

TBI PATIENTS ONLY

| | | | | |
|---|---|---|---|---|
| Mechanism of Injury | | | | |
| Pedestrian Struck by | | 10.3% | 10.7% | 10.6% |
| motor vehicle | | | | |
| MVC | | 25.6% | 26.1% | 26.0% |
| Fall >3 ft or >5 stairs | | 11.1% | 11.9% | 11.7% |
| Other fall | | 26.5% | 17.8% | 20.0% |
| Assault | | 15.4% | 18.7% | 17.8% |
| Struck by/against | | 3.4% | 4.7% | 4.4% |
| Pedal cycle without helmet | | 1.7% | 1.5% | 1.5% |
| Motorcycle | | 5.1% | 7.8% | 7.1% |
| Other | | 0.9% | 0.9% | 0.9% |
| CT Positive | | 9.4% | 20.5% | 17.6% |
| LOC | | 0.0% | 76.3% | 56.6% |
| GCS | | | | |
| 13 | | 0.0% | 1.8% | 1.3% |
| 14 | | 0.0% | 14.8% | 11.0% |
| 15 | | 100.0% | 83.4% | 87.7% |
| Altered Mental Status | | 0.0% | 69.4% | 51.5% |
| Amnesia | | 0.0% | 73.3% | 54.4% |
| Depression | | 29.9% | 30.9% | 30.6% |
| Serum Biomarker Protein | | | | |
| Mean (SD) NSE | 5.4 (±11.6) | 9.4 (±16.5) | 10.7 (±28.9) | 10.4 (±26.3) |
| Mean (SD) NRGN | 0.9 (±3.8) | 12.5 (±31.5) | 12.4 (±28.0) | 12.4 (±28.9) |
| Mean (SD) SNCB | 704.1 (±277.2) | 377.5 (±271.4) | 417.9 (±318.4) | 406.7 (±306.3) |

*Samples with values below the LLOD were excluded for the calculation of the mean (SD) of biomarkers.

Head injured patients, regardless of ACRM status, had higher levels of NSE and NRGN, and lower levels of SNCB, versus controls. FIG. 26A of the Assignee's US Patent Application Publication US2019/0339291 shows the distributions of biomarker levels (log-transformed) comparing ACRM positive TBI patients with non-TBI control patients.

The boxplots represent the data used to build the logistic regression and random forest models to discriminate between TBI and control. Univariate relationships between controls and head injury showed significant differences (P<0.001) for all 3 biomarkers. Biomarker levels were also plotted against the actual time of injury in FIG. 26B of the Assignee's US Patent Application Publication US2019/0339291. Despite the variation in sampling time after injury, biomarker levels overall remain consistent throughout the first 24 hours. Table 3 of the Assignee's US Patent Application Publication US2019/0339291 (reproduced below) demonstrates the discriminative value of models, built with logistic regression using single and multiple biomarkers, to differentiate between head injured and control patients. For comparison, the results are presented as C-statistics (equivalent to area under the curve, AUC). The highest C-statistic (0.959) was obtained using the combination of all 3 biomarkers. As univariate analysis suggested age and sex could impact marker accuracy for determining TBI status, these were included in the model. The model of 3 markers, age and sex, yielded the greatest performance (C-statistic=0.962).

TABLE 3
Comparison of C-statistics to identify best performing
models differentiating ACRM positive patients
from controls using biomarkers, age and sex

| | | AUC | AUC |
|---|---|---|---|
| | | (model with | (model with |
| | Total | biomarkers | biomarkers, |
| Biomarkers | (n) | only) | age and sex) |
| NRGN, NSE, SNCB | 469 | 0.959 | 0.962 |
| NRGN, SNCB | 478 | 0.958 | 0.962 |
| NRGN, NSE | 526 | 0.947 | 0.955 |
| NRGN | 535 | 0.943 | 0.952 |
| NSE, SNCB | 472 | 0.827 | 0.861 |
| SNCB | 481 | 0.804 | 0.845 |
| NSE | 587 | 0.655 | 0.714 |

Results for the clinical utility analysis are shown in Table 4 of the Assignee's US Patent Application Publication US2019/0339291; (reproduced below), which demonstrates that a model built with 3 markers, age and sex, with an optimized sensitivity of 98.1%, has a specificity of 77.3% using the random forest algorithm. Positive and negative predictive values of 86.4% and 96.5%, respectively, were obtained for the top performing panel. Conversely, if a performance cut point was used so that specificity was increased to 94.7%, the sensitivity for a TBI diagnosis was 90.0% (not shown in the table).

TABLE 4
Performance of top panels by statistical methods and features (biomarkers) included)

| Method | Features Included | AUC | Sensitivity | Specificity | PPV | NPV |
|---|---|---|---|---|---|---|
| Random Forest | NRGN, NSE, SNCB, age, sex | 0.978 | 0.981 | 0.773 | 0.864 | 0.965 |
| Random Forest | NRGN, NSE, SNCB | 0.972 | 0.981 | 0.679 | 0.817 | 0.960 |
| Logistic Regression | NRGN, NSE, SNCB, age, sex | 0.962 | 0.981 | 0.689 | 0.823 | 0.960 |
| Logistic Regression | NRGN, NSE, SNCB | 0.959 | 0.981 | 0.667 | 0.812 | 0.959 |

To establish clinical relevance of a positive biomarker panel, Table 5 of the Assignee's US Patent Application Publication US2019/0339291 (reproduced below) compares ACRM positive to ACRM negative patients who were classified as TBI by the random forest model (NRGN, NSE, SNCB, age, and sex). Overall, ACRM positive patients had higher rates of dysfunction. However, despite a negative evaluation at their initial presentation, a high proportion of ACRM negative patients had adverse outcomes. For ACRM negative, TBI model positive patients, 42-44% were not fully recovered at 1, 3 or 6 months after injury (GOS-E assessment <8), compared with 58-65% of ACRM positive patients. While ACRM positive patients had higher rates of post-concussive symptoms (51-54%), defined as an ICD10PCS score <0, these still occurred in 35-38% of ACRM negative, biomarker panel positive patients, when assessed at 1, 3 and 6 months after injury. Finally, rates of moderate to severe depression, defined as a PHQ9 scores >9, occurred in 10-12% of ACRM negative panel model defined TBI patients at 90 days. Overall, ACRM negative patients, classified as TBI by the marker model, had adverse event rates that were two thirds the rates of those found in the ACRM positive cohort (the latter of which included twice the rate of CT positive patients) (See FIGS. 26A and 26B of the Assignee's US Patent Application Publication US2019/0339291.)

TABLE 5
Rates of adverse outcome in ACRM positive
and ACRM negative patients

| | | 30 | | 90 | | 180 | |
|---|---|---|---|---|---|---|---|
| | Outcome | days | # | days | # | days | # |
| Patient Type | Definition | (%) | pts | (%) | pts | (%) | pts |
| ACRM+ | GOS-E < 8 | 65.6 | 253 | 60.5 | 238 | 58.3 | 218 |
| ACRM−/ | | 42.4 | 66 | 37.3 | 59 | 44.4 | 63 |
| Model+ | | | | | | | |
| ACRM+ | ICD10PCS > 0 | 54.4 | 252 | 50.8 | 236 | 51.2 | 213 |
| ACRM−/ | | 35.4 | 65 | 36.2 | 58 | 38.3 | 60 |
| Model+ | | | | | | | |
| ACRM+ | PHQ-9 ≥ 10 | 21.1 | 251 | 19.9 | 236 | 19.6 | 209 |
| ACRM−/ | | 12.3 | 65 | 13.8 | 58 | 10.2 | 59 |
| Model+ | | | | | | | |

Patients identified as TBI with the random forest model (biomarkers, age, sex).

Finally, 6 ACRM negative patients were classified as non-TBI by the biomarker panel model. Of these, at 6 month assessment only one of the patients per outcome category was found to have moderate to severe PCS (ICD10PCS=2), incomplete recovery (GOS-E=6), or significant depressive symptoms (PHQ9=12).

As described, a biomarker panel model was developed using blood test results for the biomarkers NGRN, NSE, and SNCB that, when controlled for age and sex, objectively and prospectively identified TBI patients who will suffer higher rates of dysfunction, post-concussive symptoms, and depression. The data showed that a significant percentage of patients that meet the American College of Emergency Physicians for CT evaluation, but do not meet ACRM diagnostic criteria for mTBI due to lack of symptoms, were identified by using biomarker signatures more similar to TBI than healthy controls. In these “occult TBI” patients, who test normal for all other tests except for the biomarkers, roughly one third will experience adverse outcomes, including long term disability, failure to functionally recover, and will suffer from clinically significant depression. The biomarker analysis described in this example allows for these outcomes to be prospectively known clinically; thus, neurocognitive intervention would be able to be provided to such patients as a more timely therapeutic and treatment strategy.

In addition, the biomarker panel test could serve as a screening tool for patients presenting to the emergency department with a suspected mild TBI. In fact, an objective test of this type could provide an indication of the severity of injury in patients treated on the playing field, battlefield, or in any environment that lacks access to neuroimaging equipment.

Definitive proof of benefit for any treatment in CT negative, ACRM negative patients is precluded by the lack of any objective measure to confirm or monitor disease. The findings described in this example, namely, that a prospectively identified subset of ACRM negative patients with positive biomarkers have high rates of adverse events, may suggest alternative discharge instruction. Since patients testing positive for these biomarkers are at risk for 6 month dysfunction, avoidance of high risk activities would be a reasonable consideration. Further, in a patient at risk for post-concussive syndrome, a repeat injury should be avoided. Instructions for subsequent post-ED discharge follow-up, and with an emphasis on no return to environments having high risk for head injury, would be an appropriate treatment decision.

The subset/panel of three biomarker concentrations, when controlling for age and sex bias, had good sensitivity and specificity, and the clinical utility analysis suggests that a very high sensitivity is achievable. By defining sensitivity at >98%, a method to provide a reasonable screening tool for clinicians was identified. Since high sensitivity provides a low false negative rate, and may lead to a decrease in specificity (to only 77% in this analysis), it can ensure that the risk of a missed diagnosis is clinically unlikely. This would reassure the clinician that patients with a negative biomarker panel are at less risk for long term sequelae.

The study described in this example provides valuable assessment tools for the described patient populations, e.g., those in an ED environment and a limited number of centers. The healthy control population consisted of a greater number of females and was obtained at a different environment than the head injured population. It is therefore possible that the lack of a non-head injured trauma cohort could lead to less specificity if systemic trauma should have a similar biomarker effect. In addition, only adult cohorts were assessed for the biomarkers in the described studies; therefore, application of the data does not extend to a pediatric patient population.

Accordingly, a multi-marker panel of biomarkers was identified that, when positive, determined TBI in patients compared to controls. In addition, biomarker panel positive patients suffer higher rates of dysfunction, post-concussive symptoms, and depression. The clinical implications of the findings presented in this described study may allow the objective identification of TBI at the time of presentation, which could advantageously change the clinical trajectory for patients presenting with head injury and thus may guide the development of more timely and more effective medical and clinical interventions for patients.

### OTHER EMBODIMENTS

From the foregoing description, it will be apparent that variations and modifications may be made to the t exemplary embodiments of the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1-74. (canceled)

75. A system for determining prognostic risk of having or developing brain injury related symptoms after a head impact, the system comprising:
a normative database of at least one result selected from the group of patient assay test results, patient protected health information (PHI), patient neurocognitive test results, and patient vestibular and motor test results; and
a diagnosis and prognosis server application, including instructions stored on a non-transitory computer-readable medium executed on a server that is configured to receive patient protected health information (PHI) from a PHI smart device application, neurocognitive test results from a neurocognitive testing application, and assay results from a point-of-care assay reader, and that is configured to generate a diagnostic score and a prognostic risk score for post-acute TBI symptom categories as measures of patient outcomes based on predetermined classification criteria differentiating states of brain injury and normal, non-injured conditions based on a comparison of the at least one of the group of patient assay test results, patient protected health information (PHI), patient neurocognitive test results, and patient vestibular and motor test results in the normative database with the respective patient protected health information (PHI) received from the PHI smart device application, neurocognitive test results received from a neurocognitive testing application, and assay results received from the point-of-care assay reader.

76. The system of claim 75, wherein the diagnosis and prognosis server is configured to generate a risk strata classification of the patient, wherein the risk strata classification includes at least one classification selected from the group of a) No TBI (normal); b) TBI positive (low risk for post-acute symptoms); and c) TBI positive (high risk for post-acute symptoms) in a categorized time frame associated with the determined risk strata classification of the patient, and an outcome category.

77. The system of claim 76, wherein the outcome category includes at least one category selected from the group of headache, motor deficit, sleep disturbance, cognitive deficit, and psychological disorder.

78. The system of claim 75, wherein the diagnosis and prognosis server is configured to generate at least one recommended treatment intervention selected from the group of a pain medication regimen, physical therapy, vision therapy, sleep therapy, cognitive therapy, and psychotherapy based on the risk strata classification and the predetermined time frame.

79. The system of claim 75, wherein the assay results comprise results indicative of the presence of at least one of the group of Aldolase C (ALDOC), Brain derived neurotrophic factor (BDNF), Calcitonin Gene Related Peptide (CGRP), Endothelin 1 (ET1), Eotaxin (CCL11), Fatty Acid Binding Protein 7 (FABP7), Glial Fibrillary Acidic Protein (GFAP), Growth Associated Protein 43 (GAP-43), Intercellular Adhesion Molecule 5 (ICAM-5), Interleukin 6 (IL-6), Interleukin 8 (IL-8), Interleukin 10 (IL-10), Interleukin-33 (IL-33), Metallothionein 3 (MT3), Neurogranin (NRGN), Neurofilament heavy chain (NF-H), Neurofilament light chain (NF-L), Neurofilament medium chain (NF-M), Neuron Specific Enolase (ENO2/NSE), calcium binding protein 5100B, Oligodendrocyte Myelin Glycoprotein (OMG), Reticulon (RTN1), Synuclein alpha (SNCA), Synuclein beta (SNCB), Tau microtubule binding protein (TAU/MAPT), von Willebrand Factor (vWF), and Vascular Endothelial Growth Factor (VEGF-A, B, C or D homo or heterodimers), post-translational modifications thereof, fragments thereof, auto-antibodies thereof in a biofluids sample.

80. The system of claim 79, wherein the assay results comprise results indicative of at least one additional biomarker selected from the group of biomarkers Nos. 1-82 listed in the Table of FIG. 18 in a biofluids sample.

81. The system of claim 75, wherein the neurocognitive test results comprise at least one selected from the group of (i) at least one of brain function and performance selected from the group of balance testing, oculomotor tracking, convergence insufficiency, neurocognitive memory tasks, neurocognitive pattern finding tasks, neurocognitive reasoning tasks, and neurocognitive processing speed tasks, and (ii) at least one of validated screening metrics selected from the group of headache, motor deficit, sleep disturbance, cognitive function, and psychological state.

82. The system of claim 75, wherein the neurocognitive testing application is configured to load at least one selected from the group of a digitized patient questionnaire and a motor function testing procedure whose answers form the basis for the neurocognitive test results.

83. The system of claim 82, wherein the neurocognitive testing application comprises instructions stored on a non-transitory computer-readable medium executed on a smart device located in a geographically distinct location from a location of the server, and wherein the smart device comprises a telemedicine or remote digital user interface connected to the server via a communications network.

84. A system comprising:
a. a multi-analyte assay to detect, and optionally, measure levels of, one or more biomarkers in a biofluid sample obtained from a patient having or suspected of having traumatic brain injury (TBI), the assay being integrated in a point of care device to generate first input signals representative of the levels of the one or more biomarkers;
b. a computer processor to receive digital neurocognitive, vestibular and/or oculomotor function input for the patient that includes one or more metrics of brain function and performance, and to generate second input signals for a first classifying algorithm, including one or more of balance testing, oculomotor tracking, convergence insufficiency, and specific neurocognitive tasks including one or more of memory, pattern finding, reasoning tasks, processing speed;
c. the first classifying algorithm to differentiate states of brain injury and normal condition using the first and second input signals, together with subject age and sex as covariates, to classify the patient into a TBI or No TBI category; and
d. one or more second stratification algorithms to place a patient determined to be in the TBI category into one of at least two of the following three risk strata: i) TBI positive low risk for one or more post-acute symptoms, ii) TBI positive moderate/medium risk for one or more post-acute symptoms, and iii) TBI positive high risk for one or more post-acute symptoms.

85. The system of claim 84, wherein biofluid sample obtained from the patient is one or more selected from the group of: a blood sample, a serum sample, a plasma sample, a cerebrospinal fluid (CSF) sample, a nasal fluid sample, a saliva sample, a urine sample, a sputum sample, a secretion sample, a tear sample, a sweat sample, or an organ tissue sample.

86. The system of claim 84, wherein the one or more protein biomarkers comprise Aldolase C (ALDOC), Brain derived neurotrophic factor (BDNF), Calcitonin Gene Related Peptide (CGRP), Endothelin 1 (ET1), Eotaxin (CCL11), Fatty Acid Binding Protein 7 (FABP7), Glial Fibrillary Acidic Protein (GFAP), Growth Associated Protein 43 (GAP-43), Intercellular Adhesion Molecule 5 (ICAM-5), Interleukin 6 (IL-6), Interleukin 8 (IL-8), Interleukin 10 (IL-10), Interleukin-33 (IL-33), Metallothionein 3 (MT3), Neurogranin (NRGN), Neurofilament heavy chain (NF-H), Neurofilament light chain (NF-L), Neurofilament medium chain (NF-M), Neuron Specific Enolase (ENO2/NSE), calcium binding protein S100B, Oligodendrocyte Myelin Glycoprotein (OMG), Reticulon (RTN1), Synuclein alpha (SNCA), Synuclein beta (SNCB), Tau microtubule binding protein (TAU/MAPT), von Willebrand Factor (vWF), Vascular Endothelial Growth Factor (VEGF-A, B, C or D homo or heterodimers), Soluble suppression of tumorigenicity 2 (sST2), post-translational modifications thereof, fragments thereof, auto-antibodies thereof.

87. The system of claim 86, wherein the one or more protein biomarkers further comprise one of more of the protein biomarkers, post-translational modifications thereof, fragments thereof, auto-antibodies thereof listed in FIG. 5.

88. The system of claim 84, wherein the one or more second stratification algorithms can determine the prognostic risk of having or developing brain injury related symptoms after a head impact.

89. The system of claim 84, wherein the digital neurocognitive, vestibular and/or oculomotor function input is derived from an integrated software application that captures metrics or results from performance of digitized questionnaires, neurocognitive testing, vestibular and motor function testing procedures according to a preloaded protocol.

90. A method of testing a patient having or suspected of having traumatic brain injury (TBI), the method comprising:
receiving results of a bio-sample obtained from the patient from a multi-analyte biomarker assay;
receiving neurocognitive, vestibular and/or oculomotor functional testing results from the patient;
comparing in a processor the results from the multi-analyte biomarker assay to a normative sample of results from the multi-analyte biomarker assay;
comparing in a processor the neurocognitive, vestibular and/or oculomotor functional testing results to a normative sample of respective neurocognitive, vestibular and/or oculomotor results;
differentiating states of brain injury and normal condition using the results from the multi-analyte biomarker assay and the results from the neurocognitive, vestibular and/or the oculomotor functional testing results, together with subject age and sex as covariates, in a first classifying algorithm to classify the patient into a TBI or No TBI category; and
calculating a TBI score in one or more second stratification algorithms to place a patient determined to be in the TBI category into one of at least two of the following three risk strata: i) TBI positive low risk for one or more post-acute symptoms, ii) TBI positive moderate/medium risk for one or more post-acute symptoms, and iii) TBI positive high risk for one or more post-acute symptoms.

91. The method of claim 90, further comprising:
receiving other patient health information (PHI) and using these data as additional features in a processor as inputs to the first or second algorithm, or both, and comparing in a processor the other patient health information to a normative sample of patient health information, prior to being used as additional features in the one or more algorithms.

92. The method of claim 90, wherein the multi-analyte biomarker assay tests for the presence of one or more of the following protein biomarkers selected from the group of: Aldolase C (ALDOC), Brain derived neurotrophic factor (BDNF), Calcitonin Gene Related Peptide (CGRP), Endothelin 1 (ET1), Eotaxin (CCL11), Fatty Acid Binding Protein 7 (FABP7), Glial Fibrillary Acidic Protein (GFAP), Growth Associated Protein 43 (GAP-43), Intercellular Adhesion Molecule 5 (ICAM-5), Interleukin 6 (IL-6), Interleukin 8 (IL-8), Interleukin 10 (IL-10), Interleukin-33 (IL-33), Metallothionein 3 (MT3), Neurogranin (NRGN), Neurofilament heavy chain (NF-H), Neurofilament light chain (NF-L), Neurofilament medium chain (NF-M), Neuron Specific Enolase (ENO2/NSE), Oligodendrocyte Myelin Glycoprotein (OMG), Reticulon (RTN1), Synuclein alpha (SNCA), Synuclein beta (SNCB), Tau microtubule binding protein (TAU/MAPT), von Willebrand Factor (vWF), Vascular Endothelial Growth Factor (VEGF-A, B, C or D homo or heterodimers), Soluble suppression of tumorigenicity 2 (sST2), post-translational modifications thereof, fragments thereof, auto-antibodies thereof, or combinations thereof.

93. The method of claim 92, wherein the one or more protein biomarkers further comprise one of more biomarkers selected from the group of protein biomarkers, post-translational modifications thereof, fragments thereof, auto-antibodies thereof listed in FIG. 5.

94. The method of claim 90, wherein the functional testing results comprise at least one test result selected from the group of reaction time, cognitive processing, visual attention, task switching, executive function, memory, balance testing, oculomotor tracking, and convergence insufficiency and the neurocognitive test results are based upon the results of patient tasks associated with at least one test result selected from the group of memory, pattern finding, reasoning, and processing speed.

95. The method of claim 90, wherein the risk strata are associated with TBI symptoms including post-traumatic headaches, motor deficit, sleep disturbance, seizures, depression, anxiety, loss of cognitive function, or post-traumatic stress disorders.

96. The method of claim 90, wherein the multi-analyte assay comprises a point-of-care device.

97. The method of claim 90, wherein the bio-sample is one or more samples selected from the group of: a blood sample, a serum sample, a plasma sample, a cerebrospinal fluid (CSF) sample, a saliva sample, a urine sample, a sputum sample, a secretion sample, a tear sample, a sweat sample, or an organ tissue sample.

98. The method of claim 90 further comprising the step of:
monitoring patient prognosis or recovery at a remote location outside a hospital, outpatient site, or urgent care clinical settings using internet-connected computer or internet-connected mobile device interfaces.

99. The method of claim 98, wherein the internet-connected computer or internet-connected mobile device interfaces comprise at least part of a home-based telemedicine application.

100. A method of building a classification and stratification model for the diagnosis, prognosis and treatment of patients having or suspected of having traumatic brain injury (TBI) and treating such patients, the method comprising:
a) receiving, from an assay reader, biomarker test results of a first set of biomarkers from TBI patients having differences in biomarker levels relative to a normative database;
b) receiving, from a smart device, a first set of functional test results including at least one of the following test results: neurocognitive, vestibular, and oculomotor functional test results from TBI patients having differences in functional testing results relative to a normative database;
c) integrating the results of the biomarker test results and the functional testing results, together with subject age and sex, as covariates, to build a diagnostic algorithm to classify patients into a TBI or No TBI category;
d) building one or more stratification algorithms based upon the results of step c) and a specific TBI-related symptom to place patients determined to be in the TBI category into at least one of the following three risk strata: i) TBI positive low risk for one or more post-acute symptoms, ii) TBI positive moderate/medium risk for one or more post-acute symptoms, and iii) TBI positive high risk for one or more post-acute symptoms; and
e) treating patients placed in the TBI positive moderate/medium risk or TBI positive high risk strata for TBI.

101. The method of claim 100, wherein the step d) of building a second stratification algorithm comprises training the second stratification algorithm by determining a weighting of each covariate based upon its ability to predict a change in severity of a TBI-related symptom.

102. The method of claim 101, wherein the weighting of one of more covariates is zero.

103. The method of claim 101, wherein the TBI-related symptoms comprise one or more symptoms selected from the group of the following symptoms: post-traumatic headaches, motor deficit, sleep disturbance, seizures, depression, anxiety, loss of cognitive function, post-traumatic stress disorders, dizziness, and nausea.

104. The method of claim 101, wherein the TBI-related symptoms are determined by one or more evaluations selected from the group of the following evaluations: the Rivermead Post-Concussive Symptom Questionnaire (RPQ-16), Generalized Anxiety Disorder Questionnaire 7 questions (GAD-7), Patient Health questionnaire-9 questions (PHQ-9), PTSD Checklist for DSM-5 (PCL-5), Dizziness and Headache Inventory (DHI), Perceived Stress Scale (PSS), Convergence Insufficiency Symptom Survey (CISS), Montreal Cognitive Assessment (MoCA), Mini Mental State Exam (MMSE), Saint Louis University Mental Status Examination (SLUMS), Hopkins Verbal Learning Test-Revised (HVLT-R), and/or Glasgow Outcome Score-extended (GOS-E).

105. The method of claim 104, wherein the thresholds for abnormal symptoms are as follows:
for Rivermead, scores of 3 or more;
for GAD-7, scores of 5 or more, with 10 or more being moderate to severe anxiety symptoms;
for PHQ-9, scores of 5 or more, with scores of 10 or more being moderate to severe depressive symptoms;
for PCL-5, a score of 33 or greater is not recovered (PTSD+);
for Dizziness Handicap Inventory (DHI), a score of 16 or more indicates a subject is symptomatic;
for Perceived Stress Scale (PSS), a score of 0-13 is low stress, 14-26 is moderate stress symptoms, and 27-40 is high stress symptoms;
for Convergence Insufficiency Symptom Survey (CISS), a score of 21 or greater is symptomatic for convergence insufficiency and/or abnormal oculomotor symptoms;
for Montreal Cognitive Assessment (MoCA), scores of 25 or less are cognitively impaired;
for Mini Mental State Exam (MMSE), a score of 24 or lower is symptomatic for cognitive decline (cognitive deficit);
for Saint Louis University Mental Status Examination (SLUMS), a score of 26 or lower indicating cognitive impairment for subjects with at least a high school education and 24 or lower for individuals with less than a high school education;
for Hopkins Verbal Learning Test-Revised (HVLT-R), where a score of 14.5 or lower is poor recall, and 24.5 or lower for the memory score indicating poor memory performance; and
for Glasgow Outcome Scale-extended (GOS-E), scores of 6 or less are unrecovered, having ongoing disability.

106. The method of claim 100, wherein the first set of functional tests results comprises at least one test result selected from the group of: reaction time, cognitive processing, visual attention, task switching, executive function, memory, balance testing, oculomotor tracking, and convergence insufficiency.

107. The method of claim 100, wherein the first set of functional test results comprise neurocognitive test results based upon the results of patient tasks associated with at least one task selected from the group of memory, executive function, pattern finding, reasoning, and processing speed.

108. The method of claim 107, wherein the neurocognitive test results are derived from at least one test result selected from the group of the Flanker test, the Stroop Test, the Digit symbol Substitution Test, the Trail making Test, the Trails A and Trails B cognitive and executive function tests, and an immediate and delayed recall (short term memory) test.

109. The method of claim 100, further comprising:
receiving, from a smart device, patient health information (PHI) from a prior patient physiological evaluation; and
integrating the PHI as covariates into at least one of the first diagnostic algorithm and the second stratification algorithms.

110. The method of claim 100, wherein the first set of biomarkers comprise at least one biomarker selected from the group consisting of: Aldolase C (ALDOC), Brain derived neurotrophic factor (BDNF), Calcitonin Gene Related Peptide (CGRP), Endothelin 1 (ET1), Eotaxin (CCL11), Fatty Acid Binding Protein 7 (FABP7), Glial Fibrillary Acidic Protein (GFAP), Growth Associated Protein 43 (GAP-43), Intercellular Adhesion Molecule 5 (ICAM-5), Interleukin 6 (IL-6), Interleukin 8 (IL-8), Interleukin 10 (IL-10), Interleukin-33 (IL-33), Metallothionein 3 (MT3), Neurogranin (NRGN), Neurofilament heavy chain (NF-H), Neurofilament light chain (NF-L), Neurofilament medium chain (NF-M), Neuron Specific Enolase (ENO2/NSE), Oligodendrocyte Myelin Glycoprotein (OMG), Reticulon (RTN1), Synuclein alpha (SNCA), Synuclein beta (SNCB), Soluble suppression of tumorigenicity 2 (sST2), Tau microtubule binding protein (TAU/MAPT), von Willebrand Factor (vWF), Vascular Endothelial Growth Factor (VEGF-A, B, C or D homo or heterodimers), post-translational modifications thereof, fragments thereof, auto-antibodies thereof, or combinations thereof.
